SEQUENCE LISTING (60 sequences)

SEQ ID NO: 1 - DNA, 68 bp, hepatitis delta virus
  misc_feature: hepatitis delta virus (HDV) ribozyme (Avis)
  misc_feature (1)..(6): flanking sequence
  misc_feature (7)..(68): HDV ribozyme core sequence
gcuagccauggucccagccuccucgcuggcggcuagugggcaacaugcuucggcauggcgaaugggac

SEQ ID NO: 2 - DNA, 67 bp, hepatitis delta virus
  misc_feature: hepatitis delta virus (HDV) ribozyme (Schuerer)
gggtcggcatggcatctccacctcctcgcggtccgacctgggctacttcggtaggctaagggagaag

SEQ ID NO: 3 - DNA, 56 bp, hepatitis delta virus
  misc_feature: hepatitis delta virus (HDV) ribozyme (Akoopie)
gagggatagtacagagcctccccgtggctcccttggataaccaactgatactgtac

SEQ ID NO: 4 - DNA, 50 bp, Artificial Sequence
  hammerhead ribozyme
  misc_feature (1)..(10): flanking sequence
  misc_feature (11)..(45): ribozyme core sequence
  misc_feature (46)..(50): flanking sequence
ucagguuucccugaugaguccgugaggacgaaacgguacccgguaccguc

SEQ ID NO: 5 - DNA, 92 bp, Artificial Sequence
  hairpin ribozyme
ggagagagaagucaaccagagaaacacaccaacccauugcacuccggguuggugguauauuaccugguacgggggaaacuucgugguggccg

SEQ ID NO: 6 - DNA, 176 bp, Neurospora intermedia
  misc_feature: Varkud satellite (VS) ribozyme
gggaaagcuugcgaagggcgucgucgccccgagcgguaguaagcagggaacucaccuccaauuucaguacugaaauugucguagcaguugacuacuguuaugugauugguagaggcuaagugacgguauuggcguaagucaguauugcagcacagcacaagcccgcuugcgagaau

SEQ ID NO: 7 - DNA, 152 bp, Bacillus sp.
  misc_feature: glucosamine-6-phosphate synthetase (glmS) riboswitch (glmS ribozyme)
gcgccagaactggcaccauugcacuccggugccaguugacgaggugggguuuaucgagauuucggcggaugacucccgguuguucaucacaaccgcaagcuuuuacuuaaaucauuaaggugacuuaguggacaaaggugaaagugugauga

SEQ ID NO: 8 - DNA, 19 bp, Artificial Sequence
  tetracycline operator (tetO)
tccctatcagtgatagaga

SEQ ID NO: 9 - DNA, 59 bp, Artificial Sequence
  minimal cytomegalovirus (CMV) promoter
gtaggcgtgtacggtgggaggtctatataagcagagctcgtttagtgaaccgtcagatc

SEQ ID NO: 10 - DNA, 467 bp, Artificial sequence
  “Tet-On” promoter comprising 8 tetO sequences
  misc_feature (18)..(36): tetO sequence
  misc_feature (54)..(72): tetO sequence
  misc_feature (90)..(208): tetO sequence
  misc_feature (126)..(144): tetO sequence
  misc_feature (162)..(180): tetO sequence
  misc_feature (198)..(216): tetO sequence
  misc_feature (234)..(252): tetO sequence
  misc_feature (270)..(288): tetO sequence
caattgctcgagtttactccctatcagtgatagagaacgtatgtcgactttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatacgctttaggcgtgtacggtgggcgcctataaaagcagagctcgtttagtgaaccgtcagatcgcctggagcaattccacaacacttttgtcttataccaactttccgtaccacttcctaccctcgaaagtccatatggctagcctgcagggatccagatatcaagcttcacc

SEQ ID NO: 11 - DNA, 425 bp, Artificial sequence
  “Tet-On” promoter comprising 7 tetO sequences
  misc_feature (12)..(30): tetO sequences
  misc_feature (48)..(66): tetO sequences
  misc_feature (84)..(102): tetO sequences
  misc_feature (120)..(138): tetO sequences
  misc_feature (156)..(174): tetO sequences
  misc_feature (192)..(210): tetO sequences
  misc_feature (228)..(246): tetO sequences
ctcgactttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatacgctttaggcgtgtacggtgggcgcctataaaagcagagctcgtttagtgaaccgtcagatcgcctggagcaattccacaacacttttgtcttataccaactttccgtaccacttcctaccctcgaaagtccatatggctagcctgcagggatccagatatcaagcttcacc

SEQ ID NO: 12 - DNA, 794 bp, Artificial Sequence
  “T-REx” promoter comprising two tetO sequences
  misc_feature (1)..(380): CMV enhancer
  misc_feature (381)..(584): CMV promoter
  misc_feature (586)..(604): tetO sequence
  misc_feature (607)..(625): tetO sequence
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacatcacaagtttgtacaaaaaagcaggcgctagcgtttaaacttaagcttcacc

SEQ ID NO: 13 - protein, 207 aa, Artificial Sequence
  tetracycline repressor (tetR) UniProtKB/Swiss-Prot accession no.: P04483.1
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 14 - DNA, 1442 bp, Artificial Sequence
  rtTA expression cassette
  misc_feature (1)..(588): CMV
  misc_feature (696)..(1442): TetOn3G
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttctcgagcaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 15 - DNA, 18 bp, Artificial Sequence
  T7 promoter class III (core)
  misc_feature (18)..(18): G at position 18 is preferred nucleotide at the +1 position to promote efficient transcription initiation
taatacgactcactatag

SEQ ID NO: 16 - DNA, 19 bp, Artificial Sequence
  T7 promoter class III
  misc_feature (18)..(19): GG at positions 18 and 19 are preferred nucleotides at the +1 and +2 positions to promote efficient transcription initiation
taatacgactcactatagg

SEQ ID NO: 17 - DNA, 18 bp, Artificial Sequence
  T7 promoter class II (core)
  misc_feature (18)..(18): A at position 18 is preferred nucleotide at the +1 position of the gene to promote efficient transcription initiation
taatacgactcactatta

SEQ ID NO: 18 - protein, 883 aa, Enterobacteria phage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 19 - protein, 899 aa, Artificial Sequence
  NES-T7 RNA polymerase
  MISC_FEATURE (2)..(17): NES
MCPVPLQLPPLERLTLDNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 20 - skipped

SEQ ID NO: 21 - protein, 7 aa, Simian vacuolating virus 40 (SV40)
PKKKRKV

SEQ ID NO: 22 - DNA, 1416 bp, Artificial Sequence
  Methanosarcina mazei PylRS Y306A, Y384F mutant (PylRSAF)
  misc_feature (1)..(3): ATG coding for M may be missing
atggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 23 - DNA, 1416 bp, Artificial Sequence
  Methanosarcina mazei PylRS N346A, C348A mutant (PylRSAA)
atggcgtgcccggtgccgctgcagctgccgccgctggaacgcctgaccctggatgacaaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctggcctttgcccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatctctacgaacctgtaa

SEQ ID NO: 24 - DNA, 1413 bp, Artificial Sequence
  Methanosarcina mazei PylRS Y306A, N346A, C348A, Y384Fmutant (PylRSAAAF)
gcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctggcctttgcccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 25 - DNA, 69 bp, Artificial Sequence
  pyrrolysyl tRNA of Methanosarcina mazei for Amber codon (tRNAPyl,CUA)
  misc_feature (31)..(33): anticodon
ggaaacctgatcatgtagatcgaatggactctaaatccgttcagccgggttagattcccggggtttccg

SEQ ID NO: 26 - DNA, 69 bp, Artificial Sequence
  pyrrolysyl tRNA of Methanosarcina mazei for Opal codon (tRNAPyl,UCA)
  misc_feature (31)..(33): anticodon
ggaaacctgatcatgtagatcgaatggacttcaaatccgttcagccgggttagattcccggggtttccg

SEQ ID NO: 27 - DNA, 69 bp, Artificial Sequence
  pyrrolysyl tRNA of Methanosarcina mazei for Ochre codon (tRNAPyl,UUA)
  misc_feature (31)..(33): anticodon
ggaaacctgatcatgtagatcgaatggactttaaatccgttcagccgggttagattcccggggtttccg

SEQ ID NO: 28 - protein, 6 aa, Artificial Sequence
  NES motif
  MISC_FEATURE (1)..(1): Xaa is selected from Leu, Ile, Val, Phe and Met
  MISC_FEATURE (2)..(3): Xaa is selected from any canonical amino acid
  MISC_FEATURE (4)..(4): Xaa is selected from Leu, Ile, Val, Phe and Met
  MISC_FEATURE (5)..(5): Xaa is selected from any canonical amino acid
  MISC_FEATURE (6)..(6): Xaa is selected from Leu, Ile, Val, Phe and Met
XXXXXX

SEQ ID NO: 29 - protein, 7 aa, Artificial Sequence
  NES motif
  MISC_FEATURE (1)..(1): Xaa is selected from Leu, Ile, Val, Phe and Met
  MISC_FEATURE (2)..(4): Xaa is selected from any canonical amino acid
  MISC_FEATURE (5)..(5): Xaa is selected from Leu, Ile, Val, Phe and Met
  MISC_FEATURE (6)..(6): Xaa is selected from any canonical amino acid
  MISC_FEATURE (7)..(7): Xaa is selected from Leu, Ile, Val, Phe and Met
XXXXXXX

SEQ ID NO: 30 - protein, 9 aa, Artificial Sequence
  NES motif
  MISC_FEATURE (2)..(3): Xaa is selected from any canonical amino acid
  MISC_FEATURE (4)..(4): Xaa is selected from Leu, Ile, Val, Phe and Met
  MISC_FEATURE (5)..(6): Xaa is selected from any canonical amino acid
  MISC_FEATURE (8)..(8): Xaa is selected from any canonical amino acid
  MISC_FEATURE (9)..(9): Xaa is selected from Leu and Ile
LXXXXXLXX

SEQ ID NO: 31 - protein, 8 aa, Artificial Sequence
  NES motif
  MISC_FEATURE (2)..(3): Xaa is selected from any canonical amino acid
  MISC_FEATURE (4)..(4): Xaa is selected from Leu, Ile, Val, Phe and Met
  MISC_FEATURE (5)..(6): Xaa is selected from any canonical amino acid
  MISC_FEATURE (8)..(8): Xaa is selected from any canonical amino acid
LXXXXXLX

SEQ ID NO: 32 - protein, 9 aa, Artificial Sequence
  NES motif
  MISC_FEATURE (2)..(3): Xaa is selected from any canonical amino acid
  MISC_FEATURE (4)..(4): Xaa is selected from Leu, Ile, Val, Phe and Met
  MISC_FEATURE (5)..(7): Xaa is selected from any canonical amino acid
  MISC_FEATURE (9)..(9): Xaa is selected from any canonical amino acid
LXXXXXXLX

SEQ ID NO: 33 - protein, 11 aa, Artificial Sequence
  NES motif
  MISC_FEATURE (2)..(4): Xaa is selected from any canonical amino acid
  MISC_FEATURE (5)..(5): Xaa is selected from Leu, Ile, Val, Phe and Met
  MISC_FEATURE (6)..(8): Xaa is selected from any canonical amino acid
  MISC_FEATURE (10)..(10): Xaa is selected from any canonical amino acid
  MISC_FEATURE (11)..(11): Xaa is selected from Leu and Ile
LXXXXXXXLXX

SEQ ID NO: 34 - protein, 9 aa, Artificial Sequence
  NES motif
LPPLERLTL

SEQ ID NO: 35 - protein, 17 aa, Artificial Sequence
  NES motif
ACPVPLQLPPLERLTLD

SEQ ID NO: 36 - protein, 183 aa, Artificial Sequence
  HA-PKR(2-174)
  MISC_FEATURE (2)..(10): HA
  MISC_FEATURE (11)..(183): human PKR(2-174)
MYPYDVPDYAAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKS

SEQ ID NO: 37 - protein, 159 aa, Artificial Sequence
  core of dsRNA-binding domain of PKR
  MISC_FEATURE (9)..(9): restricted; not hydrophobic (i.e. K, R, H, D, E, S, T, N, Q, C, preferably , R, S, C, Q, H, (K))
  MISC_FEATURE (10)..(10): restricted; not hydrophobic (i.e. K, R, H, D, E, S, T, N, Q, C, preferably, Q, (N))
  MISC_FEATURE (30)..(30): conserved
  MISC_FEATURE (32)..(32): conserved
  MISC_FEATURE (48)..(48): conserved
  MISC_FEATURE (50)..(52): conserved
  MISC_FEATURE (55)..(55): conserved
  MISC_FEATURE (59)..(59): restricted; A59 is not D; i.e. A59 is A, G, I, L, M, V
  MISC_FEATURE (66)..(66): restricted; L66 is not A; i.e. L66 is L, I, V, M
  MISC_FEATURE (122)..(122): conserved
  MISC_FEATURE (141)..(141): conserved
  MISC_FEATURE (145)..(145): conserved
  MISC_FEATURE (149)..(149): restricted; A149 is not D; i.e. A149 is A, G, I, L, M, V
FMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSE

SEQ ID NO: 38 - protein, 173 aa, Artificial Sequence
  (full) dsRNA-binding domain of PKR - M1
AGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKS

SEQ ID NO: 39 - DNA, 2769 bp, Artificial Sequence
  expression cassette for reporter gene NLS-iRFP-Flag-GFPY39->TAG-6His
  misc_feature (1)..(852): promoter
  misc_feature (949)..(984): NLS
  misc_feature (988)..(1929): iRFP
  misc_feature (1930)..(2004): 25 Helix
  misc_feature (2011)..(2034): Flag
  misc_feature (2035)..(2748): GFPY39 CDS
  misc_feature (2149)..(2151): Y39-encoding triplet
  misc_feature (2749)..(2766): 6His
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgggccgcctggaaagcaccccgccgaaaaaaaaacgcaaagtggaagatagcgcgagcgcggaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagctggcggaagcggcggccaaagaagcggcggccaaagaagcggcggccaaagaagcggcggccaaagcggcggccggtaccgattacaaagatgatgatgataaagtgagcaagggcgaggagctgttcaccggggtggtgcccatcctggtcgagctggacggcgacgtaaacggccacaagttcagcgtgtccggcgagggcgagggcgatgccacctagggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcatcaccatcaccatcactga

SEQ ID NO: 40 - DNA, 3341 bp, Artificial Sequence
  T7 RNA polymerase expression cassette for constitutive expression via CMV promoter
  misc_feature (1)..(588): CMV promoter
  misc_feature (690)..(3341): CDS
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 41 - DNA, 3494 bp, Artificial Sequence
  NES-T7 RNA polymerase expression cassette containing “T-REx” promoter comprising two tetO sequences
  misc_feature (1)..(584): promoter
  misc_feature (586)..(604): tetO
  misc_feature (607)..(625): tetO
  misc_feature (798)..(845): NES
  misc_feature (846)..(3494): CDS
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacatcacaagtttgtacaaaaaagcaggcgctagcgtttaaacttaagcttcaccatgtgcccggtgccgctgcagctgccgccgctggaacgcctgaccctggataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 42 - DNA, 3169 bp, Artificial Sequence
  NES-T7 RNA polymerase expression cassette containing “Tet-On” promoter comprising 8 tetO sequences
  misc_feature (1)..(458): promoter
  misc_feature (18)..(36): tetO
  misc_feature (54)..(72): tetO
  misc_feature (90)..(108): tetO
  misc_feature (126)..(144): tetO
  misc_feature (162)..(180): tetO
  misc_feature (198)..(216): tetO
  misc_feature (234)..(252): tetO
  misc_feature (270)..(288): tetO
  misc_feature (473)..(520): NES
  misc_feature (521)..(3169): CDS
caattgctcgagtttactccctatcagtgatagagaacgtatgtcgactttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatacgctttaggcgtgtacggtgggcgcctataaaagcagagctcgtttagtgaaccgtcagatcgcctggagcaattccacaacacttttgtcttataccaactttccgtaccacttcctaccctcgaaagtccatatggctagcctgcagggatccagatatcaagcttgccaccatgtgcccggtgccgctgcagctgccgccgctggaacgcctgaccctggataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 43 - DNA, 3389 bp, Artificial Sequence
  NES-T7 RNA polymerase expression cassette for constitutive expression via CMV promoter
  misc_feature (1)..(588): CMV promoter
  misc_feature (693)..(740): NES
  misc_feature (741)..(3389): CDS
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatgtgcccggtgccgctgcagctgccgccgctggaacgcctgaccctggataacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 44 - DNA, 2054 bp, Artificial Sequence
  PylRSAF expression cassette for constitutive expression via CMV promoter
  misc_feature (1)..(588): CMV promoter
  misc_feature (690)..(2054): CDS
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatggataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 45 - DNA, 2105 bp, Artificial Sequence
  NES-PylRSAF expression cassette for constitutive expression via CMV promoter
  misc_feature (1)..(588): CMV promoter
  misc_feature (696)..(743): NES
  misc_feature (744)..(2105): CDS
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttcaccatggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 46 - DNA, 2194 bp, Artificial Sequence
  NES-PylRSAF expression cassette containing “T-REx” promoter comprising two tetO sequences
  misc_feature (1)..(584): promoter
  misc_feature (586)..(604): tetO
  misc_feature (607)..(625): tetO
  misc_feature (785)..(832): NES
  misc_feature (833)..(2194): CDS
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacatcacaagtttgtacaaaaaagcaggcgctagacaccatggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 47 - DNA, 1883 bp, Artificial Sequence
  NES-PylRSAF expression cassette containing “Tet-On” promoter comprising 8 tetO sequences
  misc_feature (1)..(458): promoter
  misc_feature (18)..(36): tetO
  misc_feature (54)..(72): tetO
  misc_feature (90)..(108): tetO
  misc_feature (126)..(144): tetO
  misc_feature (162)..(180): tetO
  misc_feature (198)..(216): tetO
  misc_feature (234)..(252): tetO
  misc_feature (270)..(288): tetO
  misc_feature (474)..(521): NES
  misc_feature (522)..(1883): CDS
caattgctcgagtttactccctatcagtgatagagaacgtatgtcgactttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatacgctttaggcgtgtacggtgggcgcctataaaagcagagctcgtttagtgaaccgtcagatcgcctggagcaattccacaacacttttgtcttataccaactttccgtaccacttcctaccctcgaaagtccatatggctagcctgcagggatccagatatcaagcttcaccatggcgtgcccggtgccgctgcagctgccgccgctggaacgcctgaccctggatgataaaaaaccgctgaataccctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctagcaccaaatctggctaactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtttggcgacaccctggatgtcatgcacggcgacctggaactgtctagtgccgttgtgggcccaatcccgctggatcgtgagtggggtatcgacaaaccttggatcggtgcgggttttggtctggagcgtctgctgaaagtaaaacacgacttcaagaacatcaaacgtgctgcacgttccgagtcctattacaatggtatttctactaacctgtaa

SEQ ID NO: 48 - DNA, 226 bp, Artificial Sequence
  tRNAPyl expression cassette for expression via T7 promoter
  misc_feature (1)..(17): T7 promoter
  misc_feature (18)..(86): tRNA(Pyl)
  misc_feature (98)..(226): T7term
taatacgactcactataggaaacctgatcatgtagatcgaatggactctaaatccgttcagccgggttagattcccggggtttccgtttttggccgcctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 49 - DNA, 295 bp, Artificial Sequence
  tRNAPyl-HDV ribozyme expression cassette for expression via T7 promoter
  misc_feature (1)..(17): T7 promoter
  misc_feature (18)..(86): tRNA(Pyl)
  misc_feature (87)..(154): HDV
  misc_feature (167)..(295): T7term
taatacgactcactataggaaacctgatcatgtagatcgaatggactctaaatccgttcagccgggttagattcccggggtttccggctagccatggtcccagcctcctcgctggcggctagtgggcaacatgcttcggcatggcgaatgggactttaaactcgagctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 50 - DNA, 669 bp, Artificial Sequence
  tRNAPyl expression cassette containing a variant of the human U6 promoter modified to comprise 8 tetO before and two tetO sequences after the U6 promoter
  misc_feature (24)..(42): tetO
  misc_feature (60)..(78): tetO
  misc_feature (96)..(114): tetO
  misc_feature (132)..(150): tetO
  misc_feature (168)..(186): tetO
  misc_feature (204)..(222): tetO
  misc_feature (240)..(285): tetO
  misc_feature (276)..(294): tetO
  misc_feature (337)..(537): U6 promoter
  misc_feature (540)..(558): tetO
  misc_feature (568)..(586): tetO
  misc_feature (589)..(657): tRNA(Pyl)
gaattccaattgctcgagtttactccctatcagtgatagagaacgtatgtcgactttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagttccctatcagtgatagagattatatatctccctatcagtgatagagaccggaaacctgatcatgtagatcgaatggactctaaatccgttcagccgggttagattcccggggtttccgttttttggatcc

SEQ ID NO: 51 - DNA, 178 bp, Artificial Sequence
  tRNAPyl expression cassette containing a variant of the human H1 promoter modified to comprise 2 tetO sequences
  misc_feature (1)..(99): H1 promoter
  misc_feature (49)..(67): tetO
  misc_feature (77)..(95): tetO
  misc_feature (100)..(168): tRNA(Pyl)
atatttgcatgtcgctatgtgttctgggaaatcaccataaacgtgaaatccctatcagtgatagagacttataagttccctatcagtgatagagatcccggaaacctgatcatgtagatcgaatggactctaaatccgttcagccgggttagattcccggggtttccgaagctttttt

SEQ ID NO: 52 - protein, 248 aa, Artificial Sequence
  TetOn3G protein
  MISC_FEATURE: TetOn3G protein
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 53 - protein, 471 aa, Artificial Sequence
  Methanosarcina mazei PylRS Y306A, Y384F mutant (PylRSAF)_amino acid sequence
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLNFCQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 54 - protein, 471 aa, Artificial Sequence
  Methanosarcina mazei PylRS N346A, C348A mutant (PylRSAA)_amino acid sequence
MACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLAFAQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 55 - protein, 470 aa, Artificial Sequence
  Methanosarcina mazei PylRS Y306A, N346A, C348A, Y384Fmutant (PylRSAAAF)_amino acid sequence
ACPVPLQLPPLERLTLDDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLANYLRKLDRALPDPIKIFEIGPCYRKESDGKEHLEEFTMLAFAQMGSGCTRENLESIITDFLNHLGIDFKIVGDSCMVFGDTLDVMHGDLELSSAVVGPIPLDREWGIDKPWIGAGFGLERLLKVKHDFKNIKRAARSESYYNGISTNL

SEQ ID NO: 56 - DNA, 4437 bp, Artificial Sequence
  pCAGGS-T7-RNAP
  misc_feature (1)..(380): CMV enhancer
  misc_feature (382)..(659): Chicken b-actin promoter
  misc_feature (661)..(1678): Chimeric intron chicken actin
  misc_feature (1789)..(4437): T7-RNAP
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcatcggatccagatctgatccggatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 57 - protein, 515 aa, Mus musculus
  MISC_FEATURE (1)..(515): dsRNA-binding polypeptide  (uniprot: Q03963)
MASDTPGFYMDKLNKYRQMHGVAITYKELSTSGPPHDRRFTFQVLIDEKEFPEAKGRSKQEARNAAAKLAVDILDNENKVDCHTSASEQGLFVGNYIGLVNSFAQKKKLSVNYEQCEPNSELPQRFICKCKIGQTMYGTGSGVTKQEAKQLAAKEAYQKLLKSPPKTAGTSSSVVTSTFSGFSSSSSMTSNGVSQSAPGSFSSENVFTNGLGENKRKSGVKVSPDDVQRNKYTLDARFNSDFEDIEEIGLGGFGQVFKAKHRIDGKRYAIKRVKYNTEKAEHEVQALAELNHVNIVQYHSCWEGVDYDPEHSMSDTSRYKTRCLFIQMEFCDKGTLEQWMRNRNQSKVDKALILDLYEQIVTGVEYIHSKGLIHRDLKPGNIFLVDERHIKIGDFGLATALENDGKSRTRRTGTLQYMSPEQLFLKHYGKEVDIFALGLILAELLHTCFTESEKIKFFESLRKGDFSNDIFDNKEKSLLKKLLSEKPKDRPETSEILKTLAEWRNISEKKKRNTC

SEQ ID NO: 58 - protein, 552 aa, Pan troglodytes
  MISC_FEATURE (1)..(552): dsRNA-binding polypeptide  (uniprot: B9UKH7)
MAGDLSAGFFMEELNTYRQKQGVVLKYQELPNSGPPHDRRFTFRVIIDGREFPEGEGRSKKEAKNAAAKLAVEILNKEKKAVSPLVLTTTNSSEGLSMGNYIGLINRIAQKKRLTVNYEQCASGVHGPEGFHYKCKMGQKEYSIGTSSTKQEAKQLAAKLAYLQILSEETSVKSDYLSSGSFATTCDSQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDDLESSDYDPENSKNSSRSKTKCLFIQMEFCDKGTLEQWIEDRRGKKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDILFDKKEKTLLQKLLSKKPEDRPNTSEILRTLTVWKKSPEKNERHTC

SEQ ID NO: 59 - protein, 551 aa, Gorilla gorilla
  MISC_FEATURE (1)..(551): dsRNA-binding polypeptide (uniprot: G3RTN4)
MAGDLSAGFFMEELNTYRQKQGVVLKYQELPNSGPPHDRRFTFRVIIDEREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQIVSEETSVKSDYLSSGSFATTCDSQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGVDYDPETSDDYLESSDYDPENSKNSSRSKTKCLFIQMEFCDKGTLEQWIENRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 60 - DNA, 69 bp, Artificial Sequence
  pyrrolysyl tRNA of Methanosarcina mazei for unidentified codon (tRNAPyl, nnn)
  misc_feature (31)..(33): n can be a,t,g or c
ggaaacctgatcatgtagatcgaatggactnnnaatccgttcagccgggttagattcccggggtttccg

(19) United States
(12) Patent Application Publication
(10) Pub. No.: US 2025/0277249 A1
(43) Pub. Date: Sep. 4, 2025

(54) ARCHAEAL PYRROLYSYL TRNA SYNTHETASES FOR ORTHOGONAL USE

(71) Applicant: European Molecular Biology Laboratory, Heidelberg (DE)

(72) Inventors: Edward LEMKE, Mainz (DE); Christine KÖHLER, Forst (DE); Gemma Estrada GIRONA, London (GB); Florence MÜLLER, Heidelberg (DE)

(21) Appl. No.: 17/800,806

(22) PCT Filed: Feb. 18, 2021
(86) PCT No.: PCT/EP2021/054049

§ 371 Date: Aug. 18, 2022

(30) Foreign Application Priority Data
Feb. 21, 2020 (EP) 20158737.5

(51) Int. Cl. C12P 21/02; C12N 9/12; C12N 15/113; C12N 15/85

(52) U.S. Cl. CPC C12P 21/02; C12N 9/1247; C12N 15/113; C12N 15/85; C12Y 207/07006; C12N 2310/12; C12N 2800/107; C12N 2830/003; C12N 2830/60

(57) ABSTRACT

The present invention is concerned with efficient orthogonal translation systems for eukaryotic cells. Specifically, the invention relates to eukaryotic cells comprising polynucleotides encoding a prokaryotic tRNA synthetase (pRS) and an RNA molecule comprising a prokaryotic tRNA (ptRNA) and one or more than one ribozyme (termed “ptRNA-ribozyme” herein). It also relates to a method for expressing a polypeptide of interest (POI) comprising one or more non-canonical amino acid (ncAA) residues by such eukaryotic cells, and to kits useful in such method. The invention further relates to polynucleotides encoding the ptRNA-ribozyme.

### CROSS-REFERENCE TO RELATED APPLICATION(S)

This application claims the benefit of priority of EP Application No. 20158737.5, filed Feb. 21, 2020.

### SEQUENCE LISTING

The instant application contains a Sequence Listing which has been submitted in ASCII format via EFS-Web and is hereby incorporated by reference in its entirety. Said ASCII copy, created on Mar. 9, 2023, is named 05710.057US1_ST25 and is 1,23,137 bytes in size.

### FIELD OF THE INVENTION

The present invention is concerned with efficient orthogonal translation systems for eukaryotic cells. Specifically, the invention relates to eukaryotic cells comprising polynucleotides encoding a prokaryotic tRNA synthetase (pRS) and an RNA molecule comprising a prokaryotic tRNA (ptRNA) and one or more than one ribozyme (termed “ptRNA-ribozyme” herein), to a method for expressing a polypeptide of interest (POI) comprising one or more non-canonical amino acid (ncAA) residues by such eukaryotic cells, and to kits useful in such method. The invention further relates to polynucleotides encoding the ptRNA-ribozyme.

### BACKGROUND OF THE INVENTION

Genetic code expansion (GCE) is one of the most powerful tools for protein engineering, including site-specific introduction of post-translational modifications or labeling of proteins with specialized dyes to study protein structure or dynamics. Specifically, GCE allows for the translational modification of polypeptides by direct genetic encoding of ncAA residues, in particular using stop codon suppression, by means of a tRNA/aminoacyl tRNA synthetase (tRNA/RS) pair that is orthogonal to the translation machinery of the host cell. Ideal orthogonal tRNA/RS pairs show no cross-reactivity with the host's translation machinery, minimizing their impact on the housekeeping translational activity and normal physiology of the cell. Various prokaryotic (bacterial and archaeal) tRNA/RS pairs have been used to genetically encode a variety of ncAA residues in polypeptides including. e.g., engineered Methanococcus jannaschii tRNA/tyrosyl-RS, E. coli tRNA/leucyl-RS as well as Methanosarcina mazei and M. barkeri tRNA/pyrrolysyl-RS pairs. See. e.g., Chin et al., J Am Chem Soc 124:9026, 2002; Chin et al., Science 301:964, 2003; Nguyen et al., J Am Chem Soc 131:8720, 2009; Yanagisawa et al., Chem Biol 15:1187, 2008; Liu et al., Annu Rev Biochem 83:379-408, 2010; Lemke, ChemBioChem 15:1691-1694, 2014; Wan et al., Biochim Biophys Acta 1844:1059-170, 2014.

Over the years, various developments have been made to improve efficiency of GCE. These include, e.g., the use of archaeal RS which lack a nuclear localization signal (NLS) and/or comprise a nuclear export signal (NES) so as to allow for the RS to be localized predominantly in the cytoplasm (rather than in the nucleus) as described in WO 2018/069481. Further approaches for increasing GCE efficiency which have been explored include promoter engineering, better evolution of the orthogonal RS, release-factor engineering and multi-chaining of tRNAs, to name a few (for review see, e.g., Chin et al., Annu Rev Biochem 83:379-408, 2014).

Nevertheless, there is still a high demand for strategies which improve efficiency of GCE in eukaryotic cells. With more efficient GCE, the cell can express a higher amount of the POI comprising one or more ncAA residues. A higher amount of POI that is available for labeling and imaging purposes is, e.g., advantageous when polypeptides of low abundance are to be labeled. It was therefore an object of the present invention to (further) increase efficiency of GCE.

### SUMMARY OF THE INVENTION

The inventors surprisingly found that expression of the orthogonal tRNA as an RNA molecule that comprises the tRNA together with a ribozyme and the co-expression of a double-stranded RNA-(dsRNA-)binding polypeptide can increase the amount of POI expressed by GCE in eukaryotic cells. Without wishing to be bound by theory it is assumed that the dsRNA-binding polypeptide inhibits the phosphorylation of the alpha subunit of eukaryotic initiation factor 2α (eIF-2α) by protein kinase R (PKR) and thus alleviates the impairment of eIF-2α activity and protein synthesis associated with said PKR activity. Without wishing to be bound by theory, it is further assumed that the ribozyme improves tRNA processing which can be difficult in eukaryotic cells, and thus increases the amount of processed functional orthogonal tRNA that is available for GCE. An ample supply of processed functional orthogonal tRNA is essential for GCE (such as, e.g., amber suppression), in particular considering that orthogonal tRNAs may also interact with non-translational components inside the cells, like tRNA processing machinery and elongation factors.

Thus, in a first aspect, the present invention relates to a eukaryotic cell comprising:
(a) a polynucleotide encoding a prokaryotic aminoacyl tRNA synthetase (pRS), and
(b) a polynucleotide encoding a prokaryotic tRNA (ptRNA) and one or more than one ribozyme.

In a further aspect, the present invention relates to a eukaryotic cells comprising:
(a) a polynucleotide encoding a prokaryotic aminoacyl tRNA synthetase (pRS),
(b) a polynucleotide encoding a prokaryotic tRNA (ptRNA), and
(c) a polynucleotide encoding a polypeptide capable of binding to dsRNA (dsRNA-binding polypeptide) as described herein.

In the eukaryotic cells of the present invention, the pRS encoded by the polynucleotide of item (a) is capable of acylating the ptRNA, in particular acylating the ptRNA with non-canonical amino acid (ncAA), and the sequences encoding the ptRNA and the ribozyme(s) comprised by the polynucleotide of item (b) are linked such that transcription produces an RNA molecule comprising both the ptRNA and the ribozyme(s) (ptRNA-ribozyme).

The dsRNA-binding polypeptide used (such as, e.g., encoded by a polynucleotide used, or expressed) in the context of the present invention, comprises an amino acid sequence having at least 80%, at least 85%, at least 90%, at least 93%, at least 95%, at least 96%, at least 97%, at least 98%, at least 99% or 100% identity to the sequence set forth in any one of SEQ ID NOs:37, and 57-59.

In a further aspect, the present invention relates to a polynucleotide encoding an RNA molecule comprising a ptRNA and a ribozyme (ptRNA-ribozyme) as described herein.

In a further aspect, the present invention relates to a polynucleotide, or combination of two or more polynucleotides, comprising a nucleotide sequence encoding an RNA molecule comprising a ptRNA and a ribozyme (ptRNA-ribozyme) as described herein, and:
(i) a nucleotide sequence encoding a tetO-binding protein as described herein, or
(ii) a nucleotide sequence encoding a pRS as described herein, or
(ii) both of (i) and (ii).

Said two or three nucleotide sequences can be located on the same polypeptide. Or each of said two or three nucleotide sequences can be located on a separate polynucleotide (thus resulting in a combination of two or three polynucleotides, respectively). Or two of said three nucleotide sequences can be located on the same polynucleotide, while the third nucleotide sequence (i.e., the ptRNA-ribozyme-encoding sequence or the pRS-encoding sequence or the tetO-binding protein-encoding sequence) is located on a separate polynucleotide (thus resulting in combination of two polynucleotides).

In a further aspect, the present invention relates to a method for preparing a polypeptide of interest (POI) having one or more than one non-canonical amino acid (ncAA) residue in its amino acid sequence, wherein the method comprises:
(a) expressing, in a eukaryotic cell:
   a pRS and
   an RNA molecule comprising a ptRNA and a ribozyme (ptRNA-ribozyme) as described herein;
(b) expressing the POI in the eukaryotic cell in the presence of one or more than one ncAA or salt thereof corresponding to the one or more than one ncAA residue of the POI; and
(c) optionally recovering the expressed POI.

In a further aspect, the present invention relates to a method for preparing a polypeptide of interest (POI) having one or more than one non-canonical amino acid (ncAA) residue in its amino acid sequence, wherein the method comprises:
(a) expressing, in a eukaryotic cell:
   a pRS,
   a prokaryotic tRNA (ptRNA) and
   a polypeptide capable of binding to dsRNA (dsRNA-binding polypeptide) as described herein;
   and
(b) expressing the POI in the eukaryotic cell in the presence of one or more than one ncAA or salt thereof corresponding to the one or more than one ncAA residue of the POI; and
(c) optionally recovering the expressed POI.

In the methods for preparing a polypeptide of interest (POI) according to the present invention, the pRS is capable of acylating the ptRNA with the ncAA or the salt thereof, and the POI is encoded by a nucleotide sequence that comprises one or more than one selector codon encoding the one or more than one ncAA residue. Said selector codon is the reverse complement of the anticodon of the ptRNA.

Steps (a) and (b) of said methods can be carried out concomitantly or sequentially. For example, expression step (a) can be carried out first followed by expression step (b). When starting with the expression of pRS and the RNA molecule (ptRNA-ribozyme or ptRNA, respectively) according to step (a), said expression may continue when starting the expression of the POI according to step (b).

In a further aspect, the present invention relates to a kit for preparing a POI having one or more than one ncAA residue in its amino acid sequence, wherein the kit comprises one or more than one ncAA, or salt thereof, corresponding to the one or more than one ncAA residue of the POI, and:
(a) a polynucleotide, or combination of two or more polynucleotides, of the invention as described herein, or
(b) a polynucleotide encoding a dsRNA-binding polypeptide as described herein, or
(c) a eukaryotic cell of the invention as described herein.

In GCE, uncontrolled expression of the orthogonal tRNA/RS pair can lead to unwanted side effects. For example, over-excess of unused RS and/or unused tRNA might impair cell viability. The present invention solves this problem by further providing eukaryotic cells, polynucleotides, methods and kits for GCE wherein (i) the transcription of nucleotide sequence(s) encoding the tRNA (ptRNA or ptRNA-ribozyme), the pRS or both is controlled by a tetracycline-responsive promoter element (TRE), or (ii) the transcription of said nucleotide sequence(s) is catalyzed by an RNA polymerase (e.g., a T7 RNA polymerase), wherein expression of said RNA polymerase is controlled by a TRE. Thus, expression of said tRNA and/or pRS becomes inducible by the co-expression of proteins binding to (tetracycline operator (tetO) sequences within) the TRE, such as, e.g., a tetracycline repressor (tetR), a tetracycline-controlled transcriptional activator (tTA) or a reverse tTA (rtTA) and the presence (or absence) of tetracycline or a tetracycline derivative such as, e.g., doxycycline.

### BRIEF DESCRIPTION OF THE FIGURES

FIG. 1 provides a schematic view (5′ to 3′ direction) of tRNA expression cassettes for T7RNAP-controlled expression of a tRNA with and without a (HDV) ribozyme (“HDV-Rib.”).

FIG. 2 shows the results of a flow cytometry analysis of HEK293T cells carrying expression cassettes for the NLS-iRFP-Flag-GFP^{Y39->TAG}-6His amber suppression reporter, T7RNAP and the amber suppression pair tRNA^{Pyl}/PylRS^{AF}, wherein the tRNA^{Pyl} was either expressed together with a HDV ribozyme (“tRNA^{Pyl}-HDV rib.”) or without such ribozyme (“tRNA”). The HEK293T cells further carried an expression cassette for HA-tagged human PKR(2-174). Samples include controls where cells were not fed with the ncAA SCO (“−SCO”, bottom row). The numbers in the quadrants indicate the percentage of cells in the respective population calculated based on the total number of measured cells: cells without reporter gene expression (bottom left, “DN”=double negatives), cells showing only GFP fluorescence (bottom right, “GFP only”), cells expressing only iRFP without amber suppression (top left, “iRFP only”), cells expressing the full reporter due to amber suppression (top right, “DP”=double positives). See example 1. Experiment with plasmid pcDNA3.1 (containing the CMV promoter).

FIG. 3 shows the results of a flow cytometry analysis of HEK293T cells carrying expression cassettes for the NLS-iRFP-Flag-GFP^{Y39->TAG}-6His amber suppression reporter, T7RNAP and the amber suppression pair tRNA^{Pyl}/PylRS^{AF}, wherein the tRNA^{Pyl} was either expressed together with a HDV ribozyme (“tRNA^{Pyl}-HDV rib.”) or without such ribozyme (“tRNA^{Pyl}”). The HEK293T cells further carried an expression cassette for HA-tagged human PKR(2-174). Samples include controls where cells were not fed with the ncAA SCO (“−SCO”, bottom row). The numbers in the quadrants indicate the percentage of cells in the respective population calculated based on the total number of measured cells: cells without reporter gene expression (bottom left. “double negatives”), cells showing only GFP fluorescence (bottom right, “GFP only”), cells expressing only iRFP without amber suppression (top left, “iRFP only”), cells expressing the full reporter due to amber suppression (top right, “double positives”). See example 1. Experiment with plasmid pCAGGS (containing CMV enhancer with chicken actin promoter and intron).

FIG. 4a shows the results of a flow cytometry analysis of HEK Flp-In T-REx 293 cells carrying expression cassettes for the NLS-iRFP-Flag-GFP^{Y39->TAG}-6His amber suppression reporter, NES-T7RNAP, and the amber suppression pair tRNA^{Pyl}/NES-PylRS^{AF}, wherein the tRNA^{Pyl} was expressed together with a HDV ribozyme (“tRNA^{Pyl}-HDV rib.”) or without such ribozyme (“tRNA^{Pyl}”), and NES-PylRS^{AF} and NES-T7RNAP were inducibly expressed using the T-REx system (ThermoFisher Scientific). Cells were fed with the ncAA BOC (“+Boc”), and expression of NES-PylRS^{AF} and NES-T7RNAP was either induced with tetracycline (“+Tet”, left chart) or was not induced (control, right chart). The numbers in the quadrants indicate the percentage of cells in the respective population calculated based on the total number of measured cells: cells without reporter gene expression (bottom left. “DN”=double negatives), cells showing only GFP fluorescence (bottom right, “GFP only”), cells expressing only iRFP without amber suppression (top left, “iRFP only”), cells expressing the full reporter due to amber suppression (top right, “DP”=double positives). See example 2.

FIG. 4b shows the results of a flow cytometry analysis of HEK293T cells carrying expression cassettes for the NLS-iRFP-Flag-GFP^{Y39->TAG}-6His amber suppression reporter, NES-T7RNAP, and the amber suppression pair tRNA^{Pyl}/NES-PylRS^{AF}, wherein the tRNA^{Pyl} was expressed together with a HDV ribozyme (“tRNA^{Pyl}-HDV rib.”) or without such ribozyme (“tRNA^{Pyl}”), and NES-PylRS^{AF} and NES-T7RNAP were inducibly expressed using the Tet-On system. Cells were fed with the ncAA BOC (“+Boc”), and expression of NES-PylRS^{AF} and NES-T7RNAP was either induced with doxycycline (“+Dox”, left chart) or was not induced (control, right chart). The numbers in the quadrants indicate the percentage of cells in the respective population calculated based on the total number of measured cells: cells without reporter gene expression (bottom left, “DN”=double negatives), cells showing only GFP fluorescence (bottom right, “GFP only”), cells expressing only iRFP without amber suppression (top left, “iRFP only”), cells expressing the full reporter due to amber suppression (top right, “DP”=double positives). See example 2.

FIG. 5 shows the results of a flow cytometry analysis of HEK293T cells carrying expression cassettes for:

FIG. 5a: NLS-iRFP-Flag-GFP^{Y39->TAG}-6His amber suppression reporter, tetR, and the amber suppression pair tRNA^{Pyl}/NES-PylRS^{AF} wherein the expression of tRNA^{Pyl} was controlled by a tetracycline-inducible variant of the human U6 promoter comprising eight tetO sequences before the U6 promoter and two tetO sequences after the U6 promoter and expression of NES-PylRS^{AF} was controlled by the (constitutive) CMV promoter;

FIG. 5b: NLS-iRFP-Flag-GFP^{Y39->TAG}-6His amber suppression reporter, tetR, and the amber suppression pair tRNA^{Pyl}/NES-PylRS^{AF} wherein the expression of tRNA^{Pyl} was controlled by a tetracycline-inducible variant of the human H1 promoter comprising two tetO sequences and expression of NES-PylRS^{AF} was controlled by the (constitutive) CMV promoter;

FIG. 5c: NLS-iRFP-Flag-GFP^{Y39->TAG}-6His amber suppression reporter, tetR, and the amber suppression pair tRNA^{Pyl}/NES-PylRS^{AF} wherein the expression of tRNA^{Pyl} was controlled by a tetracycline-inducible variant of the human U6 promoter comprising eight tetO sequences before the U6 promoter and two tetO sequences after the U6 promoter and expression of NES-PylRS^{AF} was controlled by a tetracycline-inducible variant of the CMV promoter comprising two tetO sequences;

FIG. 5d: NLS-iRFP-Flag-GFP^{Y39->TAG}-6His amber suppression reporter, tetR, NES-T7RNAP expressed under the control of a tetracycline-inducible variant of the CMV promoter comprising two tetO sequences, and the amber suppression pair tRNA^{Pyl}/NES-PylRS^{AF} wherein tRNA^{Pyl} was expressed together with a HDV ribozyme under the control of the T7 promoter and expression of NES-PylRS^{AF} was controlled by a tetracycline-inducible variant of the CMV promoter comprising two tetO sequences;

FIG. 5e: NLS-iRFP-Flag-GFP^{Y39->TAG}-6His amber suppression reporter, tetR. NES-T7RNAP expressed under the control of a tetracycline-inducible variant of the CMV promoter comprising two tetO sequences, and the amber suppression pair tRNA^{Pyl}/NES-PylRS^{AF} wherein tRNA^{Pyl} was expressed together with a HDV ribozyme under the control of the T7 promoter and expression of NES-PylRS^{AF} was controlled by the (constitutive) CMV promoter;

FIG. 5f: NLS-iRFP-Flag-GFP^{Y39->TAG}-6His amber suppression reporter, tetR, NES-T7RNAP expressed under the control of the (constitutive) CMV promoter, and the amber suppression pair tRNA^{Pyl}O/NES-PylRS^{AF} wherein tRNA^{Pyl} was expressed together with a HDV ribozyme under the control of the T7 promoter and expression of NES-PylRS^{AF} was controlled by a tetracycline-inducible variant of the CMV promoter comprising two tetO sequences.

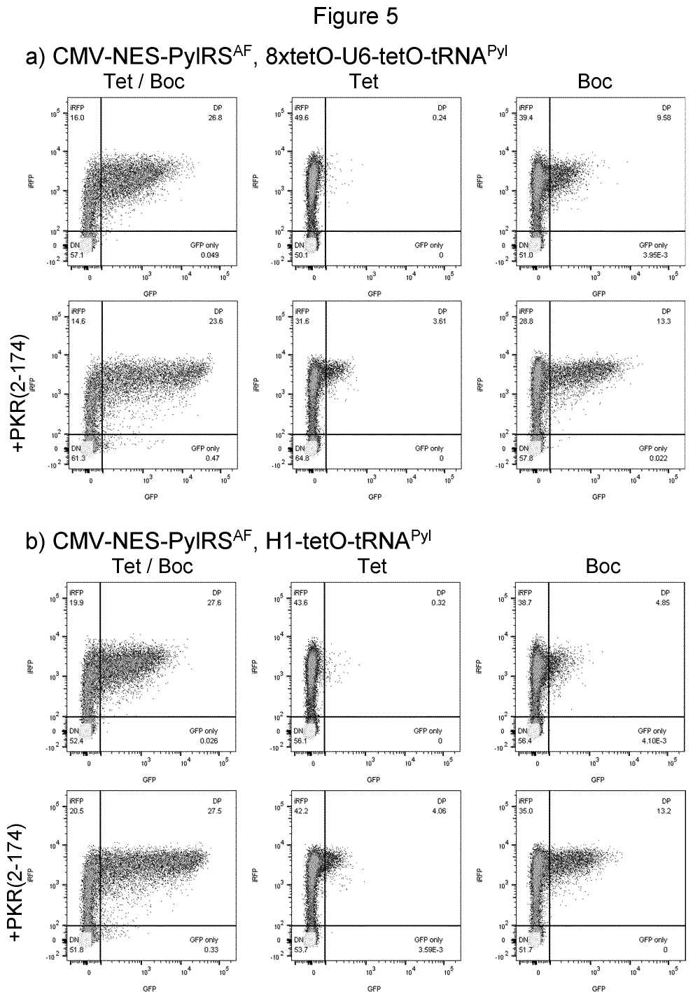

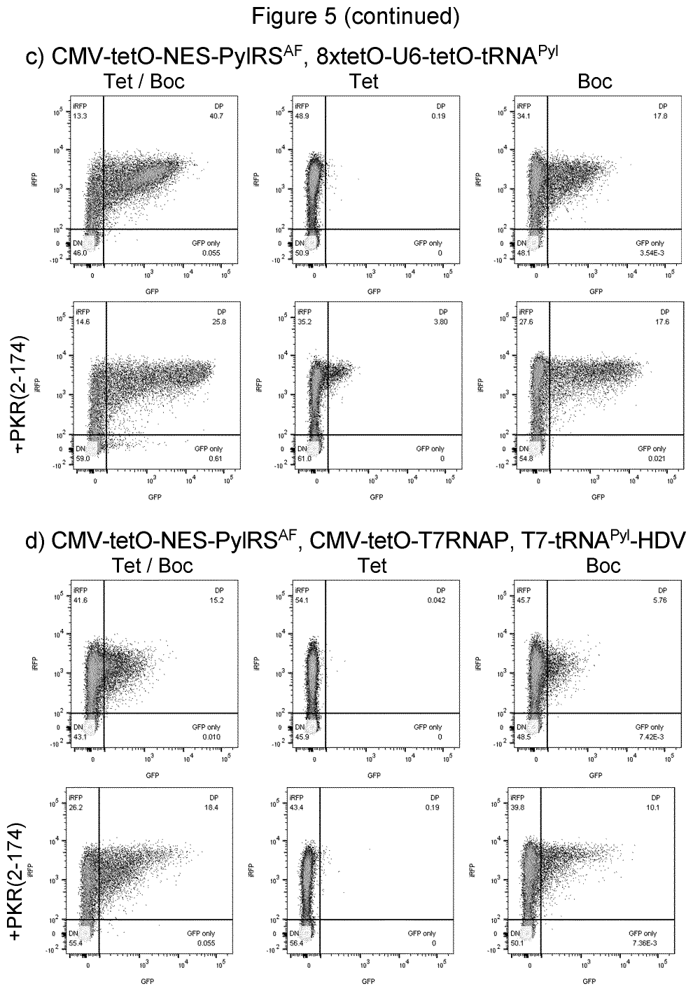

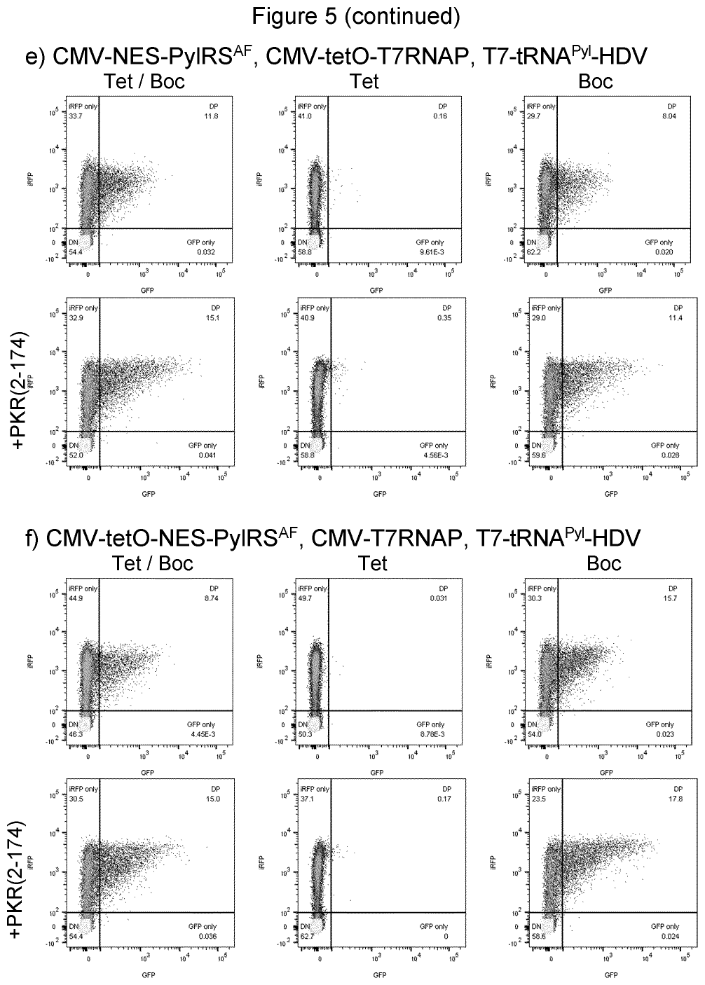

The HEK293T cells further carried an expression cassette for HA-tagged human PKR(2-174) (“+PKR(2-174)”, histograms in the bottom row of each of FIGS. 5a-f) or lacked said expression cassette (=control, histograms in the top row of each of FIGS. 5a-f). The cells were fed with the ncAA BOC (“Boc”, histograms in the left and right column of each of FIGS. 5a-f) or not (histograms in the middle column), and expression of the inducible elements (tRNA^{Pyl}, NES-PylRS^{AF} and/or NES-T7RNAP) was induced with tetracycline (“Tet” histograms in the left and middle column of each of FIGS. 5a-f) or was not induced (histograms in the right column). The numbers in the quadrants indicate the percentage of cells in the respective population calculated based on the total number of measured cells: cells without reporter gene expression (bottom left, “DN”=double negatives), cells showing only GFP fluorescence (bottom right, “GFP only”), cells expressing only iRFP without amber suppression (top left, “iRFP only”), cells expressing the full reporter due to amber suppression (top right, “DP”=double positives). See example 3.

### DETAILED DESCRIPTION OF THE INVENTION

Unless otherwise stated or required by context, singular terms shall include pluralities and plural terms shall include the singular. Thus, for example, eukaryotic cells defined as comprising “a polynucleotide encoding a pRS” include eukaryotic cells comprising one or more than one of such polynucleotide as well as eukaryotic cells wherein said polynucleotide(s) encode(s) one or more than one pRS.

If not otherwise stated, nucleotide sequences are depicted herein in the 5′ to 3′ direction. If not otherwise stated, amino acid sequences are depicted herein in the direction from N-terminus to C-terminus.

### 1. Prokaryotic Aminoacyl tRNA Synthetase/Prokaryotic tRNA (pRS/ptRNA)

The eukaryotic cells of the invention comprise a polynucleotide encoding a prokaryotic RS/tRNA (pRS/ptRNA) pair, wherein the pRS is capable of acylating the ptRNA. In particular, the pRS is capable of acylating the tRNA with an amino acid or amino acid analog (such as, e.g., compounds which differ from amino acids such that the α-amino group is replaced by a hydroxyl group and/or the carboxylic acid function forms an ester), and preferably with a non-canonical amino acid (ncAA).

The ptRNA and the pRS used in the context of the present invention are orthogonal to the translation machinery of the eukaryotic (host) cell (recombinantly) expressing said ptRNA and pRS. The term “orthogonal” as used herein indicates that the thus specified molecule (e.g., an orthogonal tRNA (O-tRNA) and/or an orthogonal RS (O-RS)) is used with reduced efficiency by the translation system (e.g., the host cell) of interest.

The term “translation system” generally refers to a set of components necessary to incorporate a naturally occurring amino acid in a growing polypeptide chain (protein). Components of a translation system include, e.g., ribosomes, tRNAs, aminoacyl tRNA synthetases, mRNA and the like. Translations systems used in the context of the present invention are preferably eukaryotic cells.

Specifically, “orthogonal” refers to the inability or reduced efficiency (such as, e.g., less than 20% efficient less than 10% efficient, less than 5% efficient, or less than 1% efficient) of an O-tRNA or an O-RS to function with the endogenous RSs or endogenous tRNAs, respectively, of the translation system of interest (wherein said orthogonal RS or orthogonal tRNA is used). For example, an O-tRNA in a translation system of interest is (amino-)acylated by any endogenous RS of the translation system with reduced or even zero efficiency, when compared to (amino-)acylation of an endogenous tRNA by the endogenous RS. For example, an O-RS (amino-)acylates any endogenous tRNA in the translation system of interest with reduced or even zero efficiency, as compared to (amino-)acylation of the endogenous tRNA by an endogenous RS. An “orthogonal translation system” is used herein to refer to a translation system using an RS/O-tRNA^{ncAA} pair that allows for introducing ncAA residues into a growing polypeptide chain.

Unless indicated differently, the terms “endogenous tRNA” and “endogenous aminoacyl tRNA synthetase” (“endogenous RS”) used therein refer to a tRNA and an RS, respectively, that was present in the cell ultimately used as translation system prior to introducing the pRS and the ptRNA, respectively, used in the context of the present invention.

The ptRNAs and pRSs used in the methods and/or fusion proteins of the invention can be naturally occurring (i.e. are ptRNAs and pRSs as found in prokaryotic organisms in nature) or can be derived by mutation of a naturally occurring ptRNA or pRS, respectively. In various embodiments, the ptRNA and pRS are derived from a prokaryote, e.g., from an eubacterium or an archaebacterium (e.g., E. coli; a Methanococcus species such as M. jannaschii; a Methanosarcina species such as M. mazei, M. barkeri. M. acetivorans, M. thermophile; a Methanococcoides species such as M. burtonii; a Desulfitobacterium species such as D. hafniense). In another embodiment, the ptRNA is a naturally occurring, or a mutant of a naturally occurring, ptRNA from a first prokaryote and the RS is derived from naturally occurring, or a mutant of a naturally occurring, pRS from a second prokaryote.

Suitable (orthogonal) ptRNA/pRS pairs can be selected from libraries of mutant ptRNAs and pRSs, e.g. based on the results of a library screening. Methods for evolving tRNA/RS pairs are described, e.g., in WO 02/085923 and WO 02/06075.

Several prokaryotic RSs have been used for genetic code expansion including the Methanococcus jannaschii tyrosyl-tRNA synthetase, E. coli tyrosyl-tRNA synthetase, E. coli leucyl-tRNA synthetase, and pyrrolysyl-tRNA synthetases from certain Methanosarcina species (such as M. mazei, M. barkeri, M. acetivorans, M. thermophila), Methanococcoides species (such as M. burtonii). Desulfitobacterium species (such as D. hafniense) or Methanomethylophilus species (such as M. alvus). Corresponding orthogonal pRS/ptRNA pairs have been used to genetically encode a variety of functionalities in polypeptides (Chin, Annu Rev Biochem 2014, 83:379-408; Chin et al., J Am Chem Soc 2001, 124:9026; Chin et al., Science 2003, 301:964; Nguyen et al., J Am Chem Soc 2009, 131:8720; Willis and Chin, Nat Chem 2018, 10:831-837); Yanagisawa et al., Chem Biol 2008, 15:1187). Such pRSs and ptRNAs can be used in the present invention.

Pyrrolysyl tRNA synthetases (PylRSs) which can be used as pRSs in the context of the present invention may be wildtype or genetically engineered PylRSs. Examples for wildtype PylRSs include, but are not limited to. PylRSs from archaebacteria and eubacteria such as Methanosarcina maize, Methanosarcina barkeri, Methanococcoides burtonii, Methanosarcina acetivorans, Methanosarcina thermophila, Methanomethylophilus alvus and Desulfitobacterium hafniense. Genetically engineered PylRSs have been described, for example, by Neumann et al. (Nat Chem Biol 2008, 4:232), by Yanagisawa et al. (Chem Biol 2008, 15:1187), and in EP2192185A1. The efficiency of genetic code expansion using PylRS can be increased by modifying the amino acid sequence of the PylRS such that it is not directed to the nucleus. To this end, the nuclear localization signal (NLS) can be removed from the PylRS or can be overridden by introducing a suitable nuclear export signal (NES). Thus, in particular embodiments of the invention, the pRS is a PylRS, or a functional fragment or mutant, as described herein that lacks the NLS and/or comprises a NES as described, e.g., in WO 2018/069481. For example, such PylRS may carry a NES, preferably inserted between (the methionine residue at) amino acid position 1 of the pRS and the amino acid position 2 of the pRS.

The term “nuclear export signal” (abbreviated as “NES”) refers to an amino acid sequence which can direct a polypeptide containing it (such as a NES-containing pRS of the invention or a NES-T7RNAP as used herein) to be exported from the nucleus of a eukaryotic cell. Said export is believed to be mostly mediated by Crm1 (chromosomal region maintenance 1, also known as karyopherin exportin 1). NESs are known in the art. For example, the database ValidNESs (http://validness.ym.edu.tw/) provides sequence information of experimentally validated NES-containing proteins. Further, NES databases like, e.g., NESbase 1.0 (www.cbs.dtu.dk/databased/NESbase-1.0/; see Le Cour et al., Nucl Acids Res 31(1), 2003) as well as tools for NES prediction like NetNES (www.cbs.dtu.dk/services/NetNES/; see La Cour et al., La Cour et al., Protein Eng Des Sel 17(6):527-536, 2004), NESpredictor (NetNES, http://www.cbs.dtu.dk/; see Fu et al., Nucl Acids Res 41:D338-D343, 2013; La Cour et al., Protein Eng Des Sal 17(6):527-536, 2004)) and NESsential (a web interface combined with ValidNESs) are available to the public. Hydrophobic leucine-rich NESs are most common and represent the best characterized group of NESs to date. A hydrophobic leucine-rich NES is a non-conservative motif having 3 or 4 hydrophobic residues. Many of these NESs comprise the conserved amino acid sequence pattern LxxLxL (SEQ ID NO:28) or LxxxLxL (SEQ ID NO:29), wherein each L is independently selected from leucine, isoleucine, valine, phenylalanine and methionine amino acid residues, and each x is independently selected from any amino acid (see La Cour et al., Protein Eng Des Sel 17(6):527-536, 2004).

Particular NESs which are suitable as signal peptide for the pRS (in particular the PylRS) used in the present invention are known in the art (e.g., from NES databases) and described, e.g., in WO 2018/069481. In specific embodiments, the pRS used in the invention comprises a hydrophobic leucine-rich NES, in particular a NES comprising the amino acid sequence LxxLxL (SEQ ID NO:28) or LxxxLxL (SEQ ID NO:29), wherein each L is independently selected from leucine, isoleucine, valine, phenylalanine and methionine, and each x is independently selected from any amino acid; more particularly a NES comprising an amino acid sequence selected from L¹xxL²xxL¹xL³ (SEQ ID NO:30), L¹xxxL²xxL¹xL³ (SEQ ID NO:31). L¹xxL²xxxL¹xL³ (SEQ ID NO:32) and L¹xxxL²xxxL¹xL³ (SEQ ID NO:33), wherein L¹ is leucine, L² is selected from leucine, isoleucine, valine, phenylalanine and methionine, L³ is selected from leucine and isoleucine, and each x is independently selected from any amino acid. Preferably, the NES comprises the amino acid sequence LPPLERLTL (SEQ ID NO:34) which is found in the HIV-1 Rev protein or, more preferably, the amino acid sequence ACPVPLQLPPLERLTLD (SEQ ID NO:35).

A “nuclear localization signal” (abbreviated as “NLS”, also referred to in the art as “nuclear localization sequence”) is an amino acid sequence that “tags” (i.e. can direct) a polypeptide containing it (e.g., NLS-iRFP-Flag-GFP^{Y39->TAG}-6His) to be imported into the nucleus of a eukaryotic cell. Said export is believed to be mediated by binding of the NLS-containing polypeptide to importin (also known as karyopherin) so as to form a complex that moves through a nuclear pore. NLSs are known in the art. A multitude of NLS databases and tools for NLS prediction are available to the public, such as NLSdb (see Nair et al., Nucl Acids Res 31(1), 2003), cNLS Mapper (www.nls-mapper.aib.keio.ac.jp; see Kosugi et al., Proc Natl Acad Sci USA. 106(25):10171-10176, 2009; Kosugi et al., J Biol Chem 284(1):478-485, 2009), SeqNLS (see Lin et al., PLoS One 8(10):e76864, 2013), and NucPred (www.sbc.su.se/˜maccallr/nucpred/; see Branmeier et al., Bioinformatics 23(9):1159-60, 2007). Classical NLSs can be monopartite or bipartite. Bipartite NLSs consist of two basic amino acid clusters and a spacer of about 10 amino acids linking said dusters. A typical core sequence comprised by monopartite NLSs is KX¹X²X³ (SEQ ID NO:20), wherein X¹ and X³ are independently selected from K and R, and X² is any amino acid. Exemplary NLS include the NLSs comprising the amino acid sequence PKKKRKV (SEQ ID NO:21).

Examples of specific pRSs which can be used in the context of the present invention include, but are not limited to:
Methanococcus jannaschii tyrosyl-tRNA synthetase;
Escherichia coli tyrosyl-tRNA synthetase;
Escherichia coli leucyl-tRNA synthetase;
Methanosarcina mazei pyrrolysyl-tRNA synthetase;
Methanosarcina barkeri pyrrolysyl-tRNA synthetase;
Methanosarcina acetivorans pyrrolysyl-tRNA synthetase;
Methanosarcina thermophila pyrrolysyl-tRNA synthetase;
Methanococcoides burtonii pyrrolysyl-tRNA synthetase;
Desulfitobacterium hafniense pyrrolysyl-tRNA synthetase;
Methanomethylophilus alvus pyrrolysyl-tRNA synthetase;
   as well as functional (i.e., enzymatically active) fragments and mutants of these polypeptides. Said functional fragments and mutants may comprise at least 60%, at least 70%, at least 80%, at least 90%, at least 95%, at least 96%, at least 97%, at least 98% or at least 99% amino acid sequence identity to the aminoacyl tRNA synthetase they are derived from.

Particular examples of pRSs useful as in the present invention which are M. mazei PylRSs include, but are not limited to:
PylRS^{AF} (Methanosarcina mazei pyrrolysyl tRNA synthetase double mutant: Y306A, Y384F; Uniprot Q8PWY1; SEQ ID NO:22);
PylRS^{AA} (Methanosarcina mazei pyrrolysyl tRNA synthetase double mutant: N346A, C348A; Uniprot Q8PWY1; SEQ ID NO:23);
PylRS^{AAAF} (Methanosarcina mazei pyrrolysyl tRNA synthetase quadruple mutant: Y306A, N346A, C348A, Y384F; Uniprot Q8PWY1; SEQ ID NO:24);
   as well as functional (i.e., enzymatically active) fragments and mutants of these polypeptide segments. Said functional fragments and mutants may comprise at least 60%, at least 70%, at least 80%, at least 90%, at least 95%, at least 96%, at least 97%, at least 98% or at least 99% amino acid sequence identity to the PylRSs they are derived from.

According to particular embodiments, wild-type and mutant M. mazei PylRSs as described herein are used for aminoacylation of tRNA with ncAAs as described in WO2012/104422 or WO2015/107064. Exemplary ncAAs for this purpose include, but are not limited to, 2-amino-6-(tert-butoxycarbonylamino)hexanoic acid (BOC), 2-amino-6-(cyclooct-2-yn-1-yloxycarbonylamino)hexanoic acid (SCO), 2-amino-6-(cyclooct-2-yn-1-yloxyethoxycarbonylamino)hexanoid acid, 2-amino-6[(4E-cyclooct-4-en-1-yl)oxycarbonylamino]hexanoic acid (TCO), 2-amino-6[(2E-cyclooct-2-en-1-yl)oxycarbonylamino]hexanoic acid (TCO*), 2-amino-6-(prop-2-ynoxycarbonylamino)hexanoic acid (PrK), 2-amino-6-(9-biocyclo[6.1.0]non-4-ynylmethoxycarbonylamino)hexanoid acid (BCN) and particularly the 2S-(L-) enantiomer of these amino acids.

Functional fragments and mutants of pRS described herein are functional in that they have the acylating enzymatic activity of the parent pRS, wherein the degree of said enzymatic activity of the fragment or mutant may be higher or less than, or about the same as, that of the parent pRS. Such fragments and mutants can be characterized by a minimum degree of sequence identity as described herein.

Amino acid or nucleotide sequence identity as used herein means identity over the entire length of the thus characterized amino acid or nucleotide sequence, respectively. The percentage identity values can be determined as known in the art on the basis of BLAST alignments, blastp algorithms (protein-protein BLAST), or using the Clustal method (Higgins et al., Comput Appl. Biosci. 1989, 5(2):151-1).

Functional fragments and mutants of particular pRSs which are useful in the present invention can be obtained, e.g., by conservative amino acid substitution, i.e. the replacement of an amino acid residue with different amino acid residues having similar biochemical properties (e.g. charge, hydrophobicity and size) as known in the art. Typical examples are substitution of Leu by lie or vice versa, substitution of Asp by Glu or vice versa, substitution of Asn by Gin or vice verse, and others.

ptRNAs which can be used in the context of the present invention include, but are not limited to, pyrrolysyl tRNA of M. mazei and functional mutants thereof. Expediently, the anticodon of the ptRNA is the anticodon to a selector codon such as, e.g., the CUA anticodon to the Amber stop codon TAG, the anticodon UCA to the Opal stop codon TGA, or the anticodon UUA to the Ochre stop codon TAA. Examples for such pyrrolysyl tRNAs include, but are not limited to, those encoded by the nucleotide sequence of SEQ ID NO:25 (tRNA^{Pyl,CUA}), SEQ ID NO:26 (tRNA^{Pyl,UCA}) or SEQ ID NO:27 (tRNA^{Pyl,UUA}).

A ptRNA used in the context of the present invention can act as a suppressor tRNA which alters the reading of a messenger RNA (mRNA) in a given translation system (e.g., a eukaryotic cell of the present invention). A suppressor tRNA can read through, e.g., a stop codon, a four base codon, or a rare codon.

O-RS/O-tRNA (pRS/ptRNA) pairs used in the invention preferably have the following properties: the O-tRNA is preferentially acylated (with an ncAA) by the O-RS. In addition, the orthogonal pair functions in the translation system of interest (e.g. the eukaryotic cell of the invention) such that the O-tRNA (that has been acylated with the ncAA) is used to incorporate said amino acid or ncAA residue into the growing polypeptide chain of a POI. Incorporation occurs in a site specific manner. Specifically, the O-tRNA recognizes (specifically binds via its anticodon to) a selector codon (e.g., an Amber, Ochre or Opal stop codon) in the mRNA coding for the POI.

The term “preferentially acylates” refers to an efficiency of, e.g., about 50% efficient, about 70% efficient, about 75% efficient, about 85% efficient, about 90% efficient, about 95% efficient, or about 99% or more efficient, at which an O-RS acylates an O-tRNA with a ncAA compared to an endogenous tRNA or amino acid of a translation system of interest. The ncAA residue is then incorporated into a growing polypeptide chain with high fidelity, e.g., at greater than about 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

### 2. Ribozymes and ptRNA-Ribozyme

In one aspect of the present invention, the ptRNA is expressed in the form of an RNA molecule comprising the ptRNA sequence and one or more than one ribozyme (ptRNA-ribozyme).

The term “ribozyme” as used herein refers to an RNA molecule, or a part thereof, that is capable of site-specific self-cleavage. Specifically, a ribozyme is capable of catalyzing the site-specific cleavage of the phosphodiester bond between two ribonucleotides within the ribonucleotide sequence of (same) the ribozyme.

Ribozymes are well known in the art. Examples of ribozymes which can be used in the ptRNA-ribozyme of the present invention include, but are not limited to, a hammerhead ribozyme (e.g., a hammerhead ribozyme of SEQ ID NO:4), a hairpin ribozyme (e.g., the hairpin ribozyme of SEQ ID NO:5), a Varkud satellite (VS) ribozyme (e.g., the VS ribozyme of SEQ ID NO:6), a glucosamine-6-phosphate synthetase (glmS) riboswitch (e.g., the glmS riboswitch of SEQ ID NO:7) and a hepatitis delta virus (HDV) ribozyme (e.g., a HDV ribozyme of SEQ ID NO:1, 2 or 3). Information on these ribozymes can be found, e.g., in Ferré-D'Amaré et al. (Cold Spring Harb Perspect Biol 2010, 2:a003574), Cochrane et al. (Chem Biol 2007, 14(1):97-105) and Schürer et al. (Nucl Acids Res 2002, 30(12):e56). In preferred embodiments of the invention, the ribozyme is a HDV ribozyme of SEQ ID NO:1.

The RNA comprising the ptRNA and the ribozyme used in the present invention comprises one or more than one ribozyme which is/are located relative to the ptRNA such that the ribozyme-catalyzed site-specific (self-)cleavage of the RNA releases the 5′-end and/or the 3′-end of the ptRNA. Thus, said ribozyme-catalyzed (self-)cleavage yields an RNA molecule comprising the ptRNA sequence wherein the 5′-end and/or the 3′-end of the ptRNA sequence coincides with the 5′-end or 3′-end, respectively, of said RNA molecule. In a particular embodiment, ribozyme-catalyzed (self-)cleavage of the RNA comprising the ptRNA and the ribozyme yields an RNA molecule that consists of the ptRNA. In a preferred embodiment, the ribozyme is a HDV ribozyme, in particular a HDV ribozyme of SEQ ID NO:1, that is directly (i.e. without any interjacent ribonucleotides) covalently linked (fused) to the 3′-end of the ptRNA so as to form a continuous ptRNA-ribozyme fusion.

### 3. Inducible and Constitutive Expression of ptRNA and/or pRS

As a further aspect, the present invention provides eukaryotic cells, methods for preparing a POI and related subject matter as described herein, wherein the expression of the ptRNA (or the ptRNA-ribozyme) or the pRS or both is inducible (in contrast to constitutive). Specifically, said inducibility is achieved in that transcription of the nucleotide sequences encoding the pRS or the ptRNA/ptRNA-ribozyme or both is controlled (directly) by a tetracycline-responsive promoter element or its transcription is catalyzed by an RNA polymerase, wherein expression of said RNA polymerase is controlled by a tetracycline-responsive promoter element.

Suitable systems for TRE-regulated transcription are well known in the art and have become standard tools for inducible gene expression. See, e.g., Gossen and Bujard, Proc Natl Acad Sci USA. 1992, 89(12):5547-5551; Yao et al., Hum Gene Ther. 1998, 9(13):1939-1950; Das et al., Curr Gene Ther, 2016, 16(3):156-167.

The term “tetracycline-responsive promoter element” (abbreviated “tetracycline response element” or “TRE”) refers to a promoter sequence comprising one, or more (such as, e.g., two, three, four, five, six, seven or more, for instance two or seven) tetracycline operator (tetO) sequences. A tetO is a polynucleotide segment having the sequence of SEQ ID NO:8 or a functional variant thereof that is capable of binding to a tetO-binding protein such as, e.g., a tetracycline repressor (tetR), tetracycline-controlled transcriptional activator (tTA) or a reverse tTA (rtTA) protein. A TRE may further comprise at least part of the minimal CMV promoter sequence set forth in SEQ ID NO:9.

In the context of the present invention, the transcription of a TRE-controlled sequence is in particular the transcriptional synthesis of a herein described RNA molecule (ptRNA or ptRNA-ribozyme), the transcription of the pRS-encoding sequence, or the transcriptional synthesis of the RNA polymerase (e.g., T7 RNA polymerase) catalyzing the transcriptional synthesis of a herein described ptRNA or ptRNA-ribozyme.

The eukaryotic cells of the invention wherein expression of one or more of the pRS, ptRNA/ptRNA-ribozyme and T7RNAP is controlled by a TRE as described herein expediently further comprise a polynucleotide encoding a tetO-binding protein (tetR, rtTA or tTA) as described herein to allow for inducing the expression said TRE-controlled coding sequences in the presence or absence, respectively, of tetracycline, doxycycline or a functional analogue thereof as described herein.

Particular examples of TREs which can be used in the context of the present invention include those comprising (or having) the sequence of any one of SEQ ID NOs:10-12. A TRE comprising (or having) the sequence of SEQ ID NO:10 or SEQ ID NO:11 is useful in combination with a rtTA, as well as for tTA. A TRE comprising (or having) the sequence of SEQ ID NO:12 is particularly useful in combination with a tetR. The tetO-binding protein and the TRE chosen to control the transcriptional synthesis of a herein described RNA molecule (ptRNA or ptRNA-ribozyme), pRS-mRNA, and/or RNA polymerase mRNA are thus expediently selected to match (bind to) each other so as to allow for inducible transcriptional control.

rtTAs and tTAs are generally known in the art, e.g., from Das et al. (Curr Gene Therapy 2016, 16:156-167) and the references cited therein. Exemplary rtTA proteins may be selected from those listed in FIG. 2B of Das et al. (supra).

rtTAs are fusion proteins comprising a first polypeptide which binds to a tetO sequence in the presence of tetracycline or a tetracycline analogue (in particular in the presence of doxycycline). Said first polypeptide is (operatively) linked to a second polypeptide which activates transcription in eukaryotic cells. The first polypeptide is preferably a mutant of the 207aa TetR selected from, e.g., the list of rtTAs shown in FIG. 2 of Das et al. (supra). The second polypeptide is preferably the 127aa transcription activation domain of the herpes simplex virus VP16 protein. The first and second polypeptide are preferably covalently linked so as to form a fusion protein, such as a fusion protein comprising the sequence of SEQ ID NO:14 and functional equivalents thereof.

An rtTA binds to (tetO sequences of) the TRE in the presence of a doxycycline or a functional analogue thereof. Said binding of the rtTA facilitates or enhances the binding of RNA polymerase and thereby the transcription of the TRE-controlled sequence. In such so-called “Tet-On” system, transcription of the TRE-controlled sequence is thus induced in the presence of an rtTA by adding doxycycline, or a functional analogue thereof. Accordingly, methods of the present invention, wherein transcription (and thus expression) of one or more of the pRS, ptRNA/ptRNA-ribozyme and T7RNAP is controlled by such Tet-On system, expediently further comprise the steps of:
expressing an rtTA in the eukaryotic cell and
contacting said cell with doxycycline, or a functional analogue thereof (thereby inducing transcription of the TRE-controlled sequence).

The rtTA is expressed in the cell at least concomitantly with (i.e., prior to and/or concomitantly with) the expression of the TRE-controlled sequence.

tTAs are fusion proteins comprising a first polypeptide which binds to a tetO sequence in the absence of tetracycline, doxycycline or a functional analogue thereof. Said first polypeptide is (operatively) linked to a second polypeptide which activates transcription in eukaryotic cells. The first polypeptide is preferably the 207aa TetR or a functional derivative thereof. The second polypeptide is preferably the 127aa transcription activation domain of the herpes simplex virus VP16 protein. The first and second polypeptides are preferably covalently linked so as to form a fusion protein.

A tTA binds to (tetO sequences of) the TRE in the absence (or at reduced concentrations) of a tetracycline, doxycycline or a functional analogue thereof. Said binding of the tTA facilitates or enhances the binding of RNA polymerase and thereby the transcription of the TRE-controlled sequence. In such so-called “Tet-Off” system, transcription of the TRE-controlled sequence is thus induced in the presence of a tTA by removing (or reducing the concentration of) previously present tetracycline, doxycycline, or a functional analogue thereof. Accordingly, methods of the present invention, wherein transcription (and thus expression) of one or more of the pRS, ptRNA/ptRNA-ribozyme and T7RNAP is controlled by such Tet-Off system, expediently further comprise the steps of:
expressing an tTA in the eukaryotic cell and
keeping said cell prior to the expression of the TRE-controlled sequence in contact with tetracycline, doxycycline, or a functional analogue thereof (thereby suppressing the binding of tTA to the TRE and thus the (trans-)activation of the transcriptional synthesis of said TRE-controlled sequence) and then reducing the concentration of, or preferably removing, said tetracycline, doxycycline, or analogue thereof so as to induce transcription of the TRE-controlled sequence.

The tTA is expressed in the cell at least concomitantly with (i.e., prior to and/or concomitantly with) the expression of the TRE-controlled sequence.

Tetracycline repressor (tetR) proteins are generally known in the art. See, e.g., Hillen and Berens, Annu Rev Microbiol 1994, 48:345-369. Exemplary tetR proteins which can be suitably used in the context of the present invention include polypeptides comprising (or having) the amino acid sequence set forth in SEQ ID NO:13 and functional equivalents thereof.

A tetR binds to (tetO sequences of) the TRE in the absence (or at reduced concentrations) of a doxycycline or a functional analogue thereof. Said binding of the tetR inhibits or prevents the binding of RNA polymerase and thereby the transcription of the TRE-controlled sequence. In such so-called “T-REx” system, transcription of the TRE-controlled sequence is thus induced in the presence of a tetR by adding tetracycline, doxycycline, or a functional analogue thereof. Accordingly, methods of the present invention, wherein transcription (and thus expression) of one or more of the pRS, ptRNA/ptRNA-ribozyme and T7RNAP is controlled by such T-REx system, expediently further comprise the steps of:
expressing a tetR in the eukaryotic cell and
contacting said cell with tetracycline, doxycycline, or a functional analogue thereof (thereby inducing transcription of the TRE-controlled sequence).

The tetR is expressed in the cell at least prior to (i.e., prior to, or prior to both prior to and concomitantly with) the expression of the TRE-controlled sequence.

The transcription of a herein described RNA molecule (ptRNA or ptRNA-ribozyme) and/or the transcription of the pRS-encoding sequence can be controlled by a constitutive promoter. Constitutive promoters and the RNA polymerases binding to such promoters are well known in the art and include eukaryotic promoters such as, for example, the CMV promoter (from human cytomegalovirus), SV40 promoter (from simian vacuolating virus 40), U6 promoter (from the human small U6 small nuclear promoter), and H1 promoter (from the human polymerase III RNA promoter), as well as prokaryotic promoters such as, for example, the T7 promoter (from T7 bacteriophage), the Sp6 promoter (from Sp6 bacteriophage).

Transcription of a herein described RNA molecule (ptRNA or ptRNA-ribozyme) and/or the transcription of the pRS-encoding sequence that is controlled by a constitutive promoter transcription can be constitutive or inducible. Constitutive transcription can be achieved in particular if the RNA polymerase which catalyzes transcription via said promoter is constitutively expressed in the eukaryotic cell. Alternatively, transcription can be inducible if the RNA polymerase which catalyzes transcription via said promoter is inducibly expressed in the eukaryotic cell, for example under the (direct) control of a TRE. In particular embodiments, wherein expression of the herein described RNA molecule (ptRNA or ptRNA-ribozyme) and/or the transcription of the pRS-encoding sequence is controlled by a constitutive promoter, and is inducible by inducible expression of the RNA polymerase which catalyzes transcription via said promoter, the constitutive promoter is a prokaryotic promoter, in particular a promoter which requires an RNA polymerase that does not naturally occur in eukaryotic cells. Particular examples include the Sp6 promoter and, preferably, the T7 promoter.

The term “T7 promoter” refers to a region of DNA that initiates transcription of a particular gene by a T7 RNA polymerase (17RNAP). According to particular embodiments, the T7 promoter used in the context of the present invention comprises (or consists of) a nucleotide sequence having at least 80%, at least 85%, at least 90%, at least 95%, at least 96%, at least 97%, at least 98%, at least 99% or 100% identity to the sequence set forth in any one of SEQ ID NOs:15-17. In preferred embodiments of invention, the T7 promoter comprises the amino acid sequence of SEQ ID NO:15 or 16.

In preferred embodiments of the invention, where transcription (and thus expression) of one or both of the pRS and the ptRNA or ptRNA-ribozyme, respectively, is controlled by a constitutive promoter, said constitutive promoter is a T7 promoter and preferably comprises (or consists of) the nucleotide sequence of any one of SEQ ID NOs:15-17. In embodiments where a T7 promoter is used, the eukaryotic cell expediently further expresses a T7RNAP. Said T7RNAP may be constitutively expressed or inducibly expressed. e.g. under the (direct) control of a TRE.

A “17 RNA polymerase” (T7RNAP) is a protein that binds to a T7 promoter and catalyzes transcription of sequences downstream of the T7 promoter. The amino acid sequence of the naturally occurring T7RNAP is known in the art, see, e.g., UniProtKB/Swiss-Prot accession no.: P00573.2. According to particular embodiments, the T7RNAP used in the context of the present invention comprises an amino acid sequence having at least 80%, at least 85%, at least 90%, at least 95%, at least 96%, at least 97%, at least 98%, at least 99% or 100% identity to the sequence set forth in SEQ ID NO:18 or the sequence set forth in SEQ ID NO:19 (includes a NES covering amino acid positions 2-17).

To direct its localizations within the eukaryotic cell, the T7RNAP (or other RNA polymerase recombinantly expressed in the eukaryotic cell) may carry a signal peptide, in particular at its N-terminus, e.g., inserted between amino acid positions 1 and 2 of the RNA polymerase. In one embodiment, the T7RNAP may carry a nuclear localization signal (NLS) or a nuclear export signal (NES) each as defined herein above (see section 1). The amino acid sequence of such NES-T7RNAP is set forth in SEQ ID NO:19. In another embodiment, the T7RNAP may not carry such nuclear localization or export tag/signal.

### 4. dsRNA-Binding Polypeptides

One aspect of the present invention provides eukaryotic cells and methods for preparing a POI as described herein, wherein the eukaryotic cells comprise polynucleotides encoding a ptRNA/pRS pair (wherein the ptRNA can be expressed as ptRNA-ribozyme) and further comprise a polynucleotide encoding a dsRNA-binding polypeptide, and wherein the method for preparing a POI comprises expressing such ptRNA/pRS pair in a eukaryotic cell and expressing a dsRNA-binding polypeptide.

The dsRNA-binding polypeptide used in the present invention is a polypeptide that is capable of binding to a double stranded RNA molecule (dsRNA) having 30 or more, preferably 80 or more base pairs, and wherein the binding is not dependent on the particular nucleotide sequence of the dsRNA. Such binding is conferred by the dsRNA-binding domain of protein kinase R (PKR) which comprises two dsRNA-binding motifs (each containing an α-β-β-β-α fold) flanking a linker region. A preferred dsRNA-binding polypeptide used in the present invention is a polypeptide comprising the amino acid sequence of SEQ ID No 36.

Accordingly, the dsRNA-binding polypeptide used in the present invention comprises, or consists of, an amino acid sequence having at least 80% (such as at least 85%, at least 90%, at least 93%, at least 95%, at least 96%, at least 97%, at least 98%, at least 99% or 100%) identity to the sequence set forth in any one of SEQ ID NOs:37, and 57-59. More preferably, the dsRNA-binding polypeptide used in the present invention comprises, or consists of, an amino acid sequence having at least 80% (such as at least 85%, at least 90%, at least 93%, at least 95%, at least 96%, at least 97%, at least 98%, at least 99% or 100%) identity to the sequence set forth in any one of SEQ ID NOs:38, and 57-59.

Expediently, the dsRNA-binding polypeptide lacks a functional PKR kinase domain and preferably is incapable of phosphorylating eukaryotic initiation factor 2α (eIF-2α).

In the methods of the invention for preparing a POI, the dsRNA-binding polypeptide is expressed at least concomitantly with (i.e. concomitantly with or both prior to and concomitantly with) the expression of the ptRNA/pRS pair.

### 5. Polynucleotides

The invention also relates to a polynucleotide, or a combination of two or more polynucleotides, comprising:
a nucleotide sequence that encodes an RNA molecule as described herein,
wherein said RNA comprises a ptRNA and a ribozyme (ptRNA-ribozyme), and, optionally:
(i) a nucleotide sequence encoding a tetO-binding protein as described herein, or
(ii) a nucleotide sequence encoding a pRS as described herein, or
(ii) both of (i) and (ii);
   and/or (a) nucleotide sequence(s) complementary to said encoding nucleotide sequence(s).

The invention further relates to a polynucleotide, or a combination of two or more polynucleotides, comprising:
a nucleotide sequence that encodes a dsRNA-binding polypeptide as described herein,
   and:
(i) a nucleotide sequence that encodes a ptRNA or a ptRNA-ribozyme as described herein, or
(ii) a nucleotide sequence encoding a pRS as described herein, or
(iii) both of (i) and (ii),
   and, optionally:
a nucleotide sequence encoding a tetO-binding protein as described herein;
   and/or (a) nucleotide sequence(s) complementary to said encoding nucleotide sequence(s).

Unless otherwise stated or required by context, the term “polynucleotide”, “polynucleotide molecule” or “nucleic acid molecule” as used herein refers to polymers of nucleotides as well as ribonucleotides, i.e. refers to both nucleic acids as well as ribonucleic acids such as single-stranded or double-stranded DNA and RNA molecules, including cDNA and mRNA.

Unless otherwise stated or required by context, the expression “nucleotide sequence encoding [an expression product]” is intended to refer to a sequence directly encoding said expression product and/or to a nucleotide sequence complementary thereto.

The encoding nucleotide sequences which are specified to be comprised by a polynucleotide, or a combination of two or more polynucleotides, of the present invention, can be located on the same polypeptide or can be distributed over two or more different polynucleotides thus resulting in a combination of said different polypeptides (for example each encoding nucleotide sequence can located on a different polypeptide of the combination).

The invention further relates to, in particular recombinant, expression constructs or expression cassettes, containing, under the genetic control of regulatory nucleotide sequences the (specified encoding) nucleotide sequence of the polynucleotide, or combination of polynucleotides, of the invention as described herein.

In one aspect of the invention, the expression cassette of the invention may comprise a nucleotide sequence encoding a ptRNA-ribozyme as described herein. In said aspect of the invention, said expression cassette may optionally further comprise, or may optionally be combined with one or more further expression cassettes comprising:
(i) a nucleotide sequence encoding a tetO-binding protein as described herein, or
(ii) a nucleotide sequence encoding a pRS as described herein, or
(ii) both of (i) and (ii).

In a further aspect of the invention, the expression cassette of the invention may comprise a nucleotide sequence encoding a dsRNA-binding polypeptide as described herein. In said aspect of the invention, said expression cassette may further comprise, or may optionally be combined with one or more further expression cassettes comprising:
(i) a nucleotide sequence that encodes a ptRNA or a ptRNA-ribozyme as described herein, or
(ii) a nucleotide sequence encoding a pRS as described herein, or
(iii) both of (i) and (ii).

The invention also relates to, in particular recombinant, vectors, comprising one or more than one of these expression cassettes or combination of expression cassettes (expression vectors).

An expression cassette comprises the nucleotide sequence encoding the expression product is functionally linked to a promoter sequence located 5′ (upstream) thereof, and typically a terminator sequence (e.g., a T7 terminator sequence) located 3′ (downstream) of said encoding sequence, and optionally further regulatory elements. Examples of such further regulatory elements include, but are not limited to, targeting sequences, enhancers, polyadenylation signals, selectable markers, amplification signals, replication origins and the like. Suitable regulatory sequences are described for example in Goeddel, Gene Expression Technology: Methods in Enzymology 185, Academic Press, San Diego, CA (1990).

A “functional” linkage of elements of polynucleotides (e.g., expression cassettes), such as promotor, encoding sequence, terminator, regulators, etc., means that these elements are arranged such that the encoding sequence can be transcribed and the optional regulatory elements can perform their regulation of said transcription. This can be achieved by a direct linkage of the elements in one and the same nucleic acid molecule. However, such direct linkage is not necessarily required. Genetic control sequences, for example enhancer sequences, can even exert their function on the target sequence from more remote positions or even from other DNA molecules. Arrangements are preferred in which the nucleic acid sequence to be transcribed is positioned downstream (i.e. at the 3′-end of) the promoter sequence, so that the two sequences are joined together covalently. The distance between the promoter sequence and the nucleic acid sequence to be expressed can be smaller than 200 base pairs, or smaller than 100 base pairs or smaller than 50 base pairs.

For expression in a cell, the expression cassette is advantageously inserted into an expression vector. Expression vectors are chosen according to the cell to be used for expression which makes optimal expression of the encoding nucleotide sequences in the cell possible. Vectors are well known by a person skilled in the art and are given for example in “Cloning vectors” (Pouwels P. H. et al., Ed., Elsevier, Amsterdam-New York-Oxford, 1985). Examples of expression vectors include, but are not limited to, plasmids, viral vectors (phages), e.g. SV40, CMV, baculovirus and adenovirus, transposons, IS elements, phasmids, cosmids, and linear or circular DNA. See, e.g., the book “Cloning Vectors” (Eds. Pouwels P. H. et al. Elsevier, Amsterdam-New York-Oxford, 1985, ISBN 0 444 904018). These vectors can be replicated autonomously in the (host) cell or can be replicated chromosomally. Expression vectors comprising one or more than one expression cassette of the present invention represent a further aspect of the invention.

For the expression of a POI in a eukaryotic cell according to the present invention, it is possible, e.g., to introduce a polynucleotide encoding the POI (e.g., an expression vector) into the cell. Alternatively, an existing gene of the cell can be modified so as to comprise selector codons at those amino acid positions where the POI is intended to carry ncAA residues. Methods for introducing (recombinant) polypeptide-encoding nucleic acid molecules into, or for modifying existing genes of, a cell are known in the art.

The term “expression” describes, in the context of the invention, the production of polypeptides encoded by the corresponding nucleotide sequence in a eukaryotic cell. The term “expression” is also used for the production of RNA molecules (ptRNA or ptRNA-ribozyme as described herein) which are encoded by nucleotide sequences in the eukaryotic cell.

The polynucleotides of the invention, including the expression cassettes and expression vectors of the invention can be prepared using common cloning techniques known in the art. Common recombination and cloning techniques are used, as described for example in T. Maniatis, E. F. Fritsch and J. Sambrook, Molecular Cloning: A Laboratory Manual, Cold Spring Harbor Laboratory, Cold Spring Harbor, NY (1989) and in T. J. Silhavy, M. L. Berman and L. W. Enquist, Experiments with Gene Fusions, Cold Spring Harbor Laboratory, Cold Spring Harbor, NY (1984) and in Ausubel, F. M. et al., Current Protocols in Molecular Biology, Greene Publishing Assoc. and Wiley interscience (1987).

The polynucleotides, or combinations of polynucleotides, of the invention, including expression cassettes and expression vectors of the invention, can be isolated, for example by methods known in the art.

An “isolated” polynucleotide or an “isolated” combination of polynucleotides is separated from other polynucleotides that are present in the natural source of the polynucleotide (e.g., the cell), and moreover can be essentially free of other cellular material or culture medium, when it is produced by recombinant techniques, or free of chemical precursors or other chemicals, when it is chemically synthesized.

Polynucleotides according to the invention can be isolated by standard techniques of molecular biology and the sequence information provided according to the invention. For example, cDNA can be isolated from a suitable cDNA-bank, using one of the concretely disclosed complete sequences or a segment thereof as hybridization probe and standard hybridization techniques (as described for example in Sambrook, J., Fritsch, E. F. and Maniatis, T. Molecular Cloning: A Laboratory Manual. 2nd edition, Cold Spring Harbor Laboratory, Cold Spring Harbor Laboratory Press, Cold Spring Harbor, NY, 1989). Moreover, a polynucleotide, comprising one of the disclosed sequences or a segment thereof, can be isolated by polymerase chain reaction, using the oligonucleotide primers that were constructed on the basis of this sequence. The polynucleotide thus amplified can be cloned into a suitable vector and can be characterized by DNA sequence analysis.

### 6. POIs Having One or More than One ncAA Residue

The present invention provides methods for preparing a POI having one or more than one ncAA residue. The ncAA residues of the POI are encoded by selector codons.

The term “selector codon” as used herein refers to a codon that is recognized (i.e. bound) by the ptRNA in the translation process and is not recognized by endogenous tRNAs of the eukaryotic cell. The term is also used for the corresponding codons in polypeptide-encoding sequences of polynucleotides which are not messenger RNAs (mRNAs), e.g. DNA plasmids. Preferably, the selector codon is a codon of low abundance in naturally occurring eukaryotic cells. Expediently, the anticodon of the ptRNA used in the present invention specifically binds to (hybridizes with) a selector codon within an mRNA and thus incorporates the ncAA residue site-specifically into the growing chain of the polypeptide encoded by said mRNA. The known 64 genetic (triplet) codons code for the 20 canonical amino acid residues and three stop codons. Because only one stop codon is needed for translational termination, the other two can in principle be used to encode non-proteinogenic amino acids. For example, the amber codon, UAG, has been successfully used as a selector codon in in vitro and in vivo translation systems to direct the incorporation of ncAAs. Selector codons utilized in methods of the present invention expand the genetic codon framework of the protein biosynthetic machinery of the translation system used. Specifically, selector codons include, but are not limited to: nonsense codons, such as stop codons, e.g., amber (UAG), ochre (UAA), and opal (UGA) codons; codons consisting of more than three bases (e.g., four base codons); and codons derived from natural or unnatural base pairs. For a given system, a selector codon can also include one of the natural three base codons (i.e. natural triplets), wherein the endogenous translation system does not (or only scarcely) use said natural triplet, e.g., a system that is lacking a tRNA that recognizes the natural triplet or a system wherein the natural triplet is a rare codon.

A recombinant tRNA that alters the reading of an mRNA in a given translation system such that it allows for reading through, e.g., a stop codon, a four base codon, or a rare codon (such as a ptRNA in a eukaryotic cell of the present invention), is termed suppressor tRNA. The suppression efficiency for a stop codon serving as a selector codon (e.g., the amber codon) depends upon the competition between the (aminoacylated) recombinant tRNA (ptRNA) which acts as suppressor tRNA and the release factor (e.g. RF1) which binds to the stop codon and initiates release of the growing polypeptide chain from the ribosome. Suppression efficiency of such stop codon can therefore be increased using a release factor-(e.g. RF1-)deficient strain.

A nucleotide sequence encoding a target polypeptide (also referred to herein as polypeptide of interest or POI) can comprise one or more, e.g., two or more, three or more, etc., codons (e.g. selector codons) which are the reverse complement of the anticodon comprised by the ptRNA. Conventional site-directed mutagenesis can be used to introduce said selector codon(s) at the site of interest into a nucleotide sequence so as to generate a POI-encoding nucleotide sequence.

The abbreviation “ncAA” refers generally to any non-canonical or non-natural amino acid (or amino acid residue) that is not among the 20 naturally occurring “standard” proteinogenic amino acids (R, H, K, D, E, S, T, N, Q, C, G, P, A, V, I, L, M, F, Y, W) and is also not selenocysteine or pyrrolysine. Numerous ncAAs are well known in the art. NcAAs useful in methods and kits of the present invention have been described in the prior art (for review see e.g. Liu et al., Annu Rev Biochem 83:379-408, 2010, Lemke, ChemBioChem 15:1691-1694, 2014).

The term “ncAA” also refers to amino acid analogs, e.g. compounds which differ from amino acids such that the α-amino group is replaced by a hydroxyl group (α-hydroxyl acid) and/or the carboxylic acid function forms an ester. When translationally incorporated into a polypeptide, an α-hydroxyl acid (residue) is coupled through its α-hydroxyl group to carboxylic acid function of the adjacent amino acid (analogue) residue by an ester bond. When ncAAs which are amino acid analogs wherein the carboxylic acid function forms an ester of formula —C(O)—O—R are used for preparing polypeptides in a translation system (such as a eukaryotic cell), it is believed that R is removed in situ, for example enzymatically, in the translation system prior of being incorporated in the POI. Accordingly, R is expediently chosen so as to be compatible with the translation system's ability to convert the ncAA or salt thereof into a form that is recognized and processed by the PylRS of the invention.

Particular preferred ncAAs in the context of the present invention are those which can be post-transitionally further modified. For example, ncAAs may comprise a group (herein referred to as “labeling group”) that facilitates reaction with a suitable group (herein referred to as “docking group”) of another molecule (herein termed “conjugation partner molecule”) so as to covalently attach the conjugation partner molecule to the ncAA. When an ncAA comprising a labeling group is translationally incorporated into a POI, the labeling group becomes part of the POI. Accordingly, a POI prepared according to the method of the present invention can be reacted with one or more than one conjugation partner molecule such that the conjugation partner molecules bind covalently to the (labeling groups of the) non-canonical amino acid (ncAA) residue(s) of the POI. Such conjugation reactions may be used for in situ coupling of POIs within a cell or tissue expressing the POI, or for site-specific conjugation of isolated or partially isolated POIs.

Particular useful choices for combinations of labeling groups and docking groups (of conjugation partner molecules) are those which can react by metal-free click reactions. Such click reactions include strain-promoted inverse-electron-demand Diels-Alder cycloadditions (SPIEDAC; see, e.g., Devaraj et al., Angew Chem Int Ed Engl 2009, 48:7013)) as well as cycloadditions between strained cycloalkynyl groups, or strained cycloalkynyl analog groups having one or more of the ring atoms not bound by the triple bond substituted by amino groups), with azides, nitrile oxides, nitrones and diazocarbonyl reagents (see. e.g., Sanders et al., J Am Chem Soc 2010, 133:949; Agard et al., J Am Chem Soc 2004, 126:15046), for example strain promoted alkyne-azide cycloadditions (SPAAC). Such click reactions allow for ultrafast and biorthogonal covalent site-specific coupling of ncAA labeling groups of POIs with suitable groups of coupling partner molecule.

Pairs of docking and labeling groups which can react via the above-mentioned click reactions are known in the art. Examples of suitable ncAAs comprising docking groups include, but are not limited to, the ncAAs described, e.g., in WO 20121104422 and WO 2015/107064.

Examples of particular suitable pairs of docking groups (comprised by the conjugation partner molecule) and labeling groups (comprised by the ncAA residue(s) of the POI) include but are not limited to:
(a) a docking group comprising (or essentially consisting of) a group selected from an azido group, a nitrile oxide functional group (i.e. a radical of formula, a nitrone functional group or a diazocarbonyl group, combined with a labeling group comprising (or essentially consisting of) an optionally substituted strained alkynyl group (such groups can react covalently in a copper-free strain promoted alkyne-azide cycloaddition (SPAAC));
(b) a docking group comprising (or essentially consisting of) an optionally substituted strained alkynyl group, combined with a labeling group comprising (or essentially consisting of) a group selected from an azido group, a nitrile oxide functional group (i.e. a radical of formula, a nitrone functional group or a diazocarbonyl group (such groups can react covalently in a copper-free strain promoted alkyne-azide cycloaddition (SPAAC));
(c) a docking group comprising (or essentially consisting of) a group selected from optionally substituted strained alkynyl groups, optionally substituted strained alkenyl groups and norbornenyl groups, combined with a labeling group comprising (or essentially consisting of) an optionally substituted tetrazinyl group (such groups can react covalently in a copper-free strain promoted inverse-electron-demand Diels-Alder cycloaddition (SPIEDAC)).
(d) a docking group comprising (or essentially consisting of) an optionally substituted tetrazinyl group, combined with a labeling group comprising (or essentially consisting of) a group selected from optionally substituted strained alkynyl groups, optionally substituted strained alkenyl groups and norbornenyl groups (such groups can react covalently in a copper-free strain promoted inverse-electron-demand Diels-Alder cycloaddition (SPIEDAC)).

Optionally substituted strained alkynyl groups include, but are not limited to, optionally substituted trans-cyclooctenyl groups, such as those described in. Optionally substituted strained alkenyl groups include, but are not limited to, optionally substituted cyclooctynyl groups, such as those described in WO 2012/104422 and WO 2015/107064. Optionally substituted tetrazinyl groups include, but are not limited to, those described in WO 2012/104422 and WO 2015/107064.

An azido group is a radical of formula —N₃.

A nitrone functional group is a radical of formula —C(R^{x})═N⁺(R^{y})—O⁻, wherein R^{x} and R^{y} are organic residues. e.g., residues independently selected from C₁-C₆-alkyl as described herein.

A diazocarbonyl group is a radical of formula —C(O)—CH═N₂.

A nitrile oxide functional group is a radical of formula —C≡N⁺—O⁻ or, preferably, of formula —C═N⁺(R^{x})—O—, wherein R^{x} is an organic residue, e.g., a residue selected from C₁-C₆-alkyl as described herein.

“Cyclooctynyl” is an unsaturated cycloaliphatic radical having 8 carbon atoms and one triple bond in the ring structure.

“Trans-cyclooctenyl” is an unsaturated cycloaliphatic radical having 8 carbon atoms and one double bond that is in trans configuration in the ring structure.

“Tetrazinyl” is a 6-membered monocyclic aromatic radical having 4 nitrogen ring atoms and 2 carbon ring atoms.

Unless indicated otherwise, the term “substituted” means that a radical is substituted with 1, 2 or 3, especially 1 or 2, substituent(s). In particular embodiments, these substituents can be selected independently from hydrogen, halogen, C₁-C₄-alkyl, (R^{a}O)₂P(O)O—C₁-C₄-alkyl, (R^{b}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, di-(C₁-C₄-alkyl)amino, C₂-C₅-alkenylamino, N—C₂-C₅-alkenyl-N—C₁-C₄-alkyl-amino and di-(C₂-C₅-alkenyl)amino, wherein R^{a} and R^{b} R^{a}, R^{b} are independently hydrogen or C₂-C₅-alkanoyloxymethyl.

The term halogen denotes in each case a fluorine, bromine, chlorine or iodine radical, in particular a fluorine radical.

C₁-C₄-Alkyl is a straight-chain or branched alkyl group having from 1 to 4, in particular from 1 to 3 carbon atoms. Examples include methyl and C₂-C₄-alkyl such as ethyl, n-propyl, iso-propyl, n-butyl, 2-butyl, iso-butyl and tert-butyl.

C₂-C₅-Alkenyl is a singly unsaturated hydrocarbon radical having 2, 3, 4 or 5 carbon atoms. Examples include vinyl, allyl (2-propen-1-yl), 1-propen-1-yl, 2-propen-2-yl, methallyl (2-methylprop-2-en-1-yl), 1-methylprop-2-en-1-yl, 2-buten-1-yl, 3-buten-1-yl, 2-penten-1-yl, 3-penten-1-yl, 4-penten-1-yl, 1-methylbut-2-en-1-yl and 2-ethylprop-2-en-1-yl.

C₁-C₄-Alkoxy is a radical of formula R—O—, wherein R is a C₁-C₄-alkyl group as defined herein.

C₂-C₅-Alkenoxy is a radical of formula R—O—, wherein R is C₂-C₅-alkenyl as defined herein.

C₂-C₅-Alkanoyloxy is a radical of formula R—C(O)—O—, wherein R is C₁-C₄-alkyl as defined herein.

C₁-C₄-Alkylaminocarbonyloxy is a radical of formula R—NH—C(O)—O—, wherein R is C₁-C₄-alkyl as defined herein.

C₁-C₄-Alkythio is a radical of formula R—S—, wherein R is C₁-C₄-alkyl as defined herein.

C₁-C₄-Alkylamino is a radical of formula R—NH—, wherein R is C₁-C₄-alkyl as defined herein.

Di-(C₁-C₄-alkyl)amino is a radical of formula R^{x}—N(R^{y})—, wherein R^{x} and R^{y} are independently C₁-C₄-alkyl as defined herein.

C₂-C₅-Alkenylamino is a radical of formula R—NH—, wherein R is C₂-C₅-alkenyl as defined herein.

N—C₂-C₅-alkenyl-N—C₁-C₄-alkyl-amino is a radical of formula R^{x}—N(R^{y})—, wherein R^{x} is C₂-C₅-alkenyl as defined herein and R^{y} is C₁-C₄-alkyl as defined herein.

Di-(C₂-C₅-alkenyl)amino is a radical of formula R^{x}—N(R^{y})—, wherein R^{x} and R^{y} are independently C₂-C₅-alkenyl as defined herein.

C₂-C₅-Alkanoyloxymethyl is a radical of formula R^{x}—C(O)—O—CH₂—, wherein R^{x} is C₁-C₄-alkyl as defined herein.

The ncAAs used in the context of the present invention can be used in the form of their salt. Salts of ncAAs as described herein mean acid or base addition salts, especially addition salts with physiologically tolerated acids or bases. Physiologically tolerated acid addition salts can be formed by treatment of the base form of an ncAA with appropriate organic or inorganic acids. NcAAs containing an acidic proton may be converted into their non-toxic metal or amine addition salt forms by treatment with appropriate organic and inorganic bases. The ncAAs and salts thereof described in the context of the present invention also comprise the hydrates and solvent addition forms thereof, e.g. hydrates, alcoholates and the like.

Physiologically tolerated acids or bases are in particular those which are tolerated by the translation system used for preparation of POI with ncAA residues, e.g. are substantially non-toxic to living eukaryotic cells.

NcAAs, and salts thereof, useful in the context of the present the invention can be prepared by analogy to methods which are well known in the art and are described, e.g., in the various publications cited herein.

The nature of the coupling partner molecule depends on the intended use. For example, the POI may be coupled to a molecule suitable for imaging methods or may be functionalized by coupling to a bioactive molecule. For instance, in addition to the docking group, a coupling partner molecule may comprise a group that selected from, but are not limited to, dyes (e.g. fluorescent, luminescent, or phosphorescent dyes, such as dansyl, coumarin, fluorescein, acridine, rhodamine, silicon-rhodamine, BODIPY, or cyanine dyes), molecules able to emit fluorescence upon contact with a reagent, chromophores (e.g., phytochrome, phycobilin, bilirubin, etc.), radiolabels (e.g. radioactive forms of hydrogen, fluorine, carbon, phosphorous, sulphur, or iodine, such as tritium, ¹⁸F, ¹¹C, ¹⁴C, ³²P, ³³P, ³³S, ³⁵S, ¹¹In, ¹²⁵I, ¹²³I, ¹³¹I, ²¹²B, ⁹⁰Y or ¹⁸⁶Rh), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes. X-ray probes, pH probes, IR probes, resins, solid supports and bioactive compounds (e.g. synthetic drugs). Suitable bioactive compounds include, but are not limited to, cytotoxic compounds (e.g., cancer chemotherapeutic compounds), antiviral compounds, biological response modifiers (e.g., hormones, chemokines, cytokines, interleukins, etc.), microtubule affecting agents, hormone modulators, and steroidal compounds. Specific examples of useful coupling partner molecules include, but are not limited to, a member of a receptor/ligand pair; a member of an antibody/antigen pair, a member of a lectin/carbohydrate pair, a member of an enzyme/substrate pair; biotin/avidin; biotin/streptavidin and digoxin/antidigoxin.

The ability of certain (labeling groups of) ncAA residues to be coupled covalently in situ to (the docking groups of) conjugation partner molecules, in particular by a click reaction as described herein, can be used for detecting a POI having such ncAA residue(s) within a eukaryotic cell or tissue expressing the POI, and for studying the distribution and fate of the POI. Thus, for example, the method of the present invention for preparing a POI by expression in eukaryotic cells can be combined with super-resolution microscopy (SRM) to detect the POI within the cell or a tissue of such cells. Several SRM methods are known in the art and can be adapted so as to utilize lick chemistry for detecting a POI expressed by a eukaryotic cell of the present invention. Specific examples of such SRM methods include DNA-PAINT (DNA point accumulation for imaging in nanoscale topography; described, e.g., by Jungmann et al., Nat Methods 11:313-318, 2014). dSTORM (direct stochastic optical reconstruction microscopy) and STED (stimulated emission depletion) microscopy.

### 7. Eukaryotic Cells of the Invention

Eukaryotic cells of the invention can be generated by introducing polynucleotides as described herein, in particular polynucleotide comprising nucleotide sequences encoding a pRS and encoding a ptRNA or ptRNA-ribozyme as described herein. And in one aspect, the polynucleotide(s) introduced into the eukaryotic cell further comprise a nucleotide sequence encoding a dsRNA-binding polypeptide. Optionally, the introduced polynucleotide(s) further comprise nucleotide sequences encoding a tetO binding protein and/or a constitutive RNA polymerase (e.g. a T7RNAP) as described herein. Said nucleotide sequences can be located on the same polynucleotide or distributed over two or more different polynucleotides.

Eukaryotic cells of the present invention can be selected from, but are not limited to, mammalian cells (e.g., human, mouse, rat, cynomolgus monkey cells), avian cells (e.g., chicken cells), insect cells, yeast cells and plant cells. The eukaryotic cells of the invention may be present as individual cells or may be part of a tissue (e.g. a cell in a (cultured) tissue, organ or entire organism).

A POI comprising one or more than one non-canonical amino acid (ncAA) residue in its amino acid sequence can be prepared according to the present invention using a eukaryotic cell, in particular a eukaryotic cell of the present invention. The eukaryotic cell expresses a pRS and a ptRNA which for a pair in that the pRS is capable of acylating the ptRNA. In particular embodiments, the ptRNA is expressed in the form of a ptRNA-ribozyme as described herein. The eukaryotic cell further expresses the POI in the presence of one or more than one ncAA or salt thereof corresponding to the one or more than one ncAA residue of the POI. To this end the eukaryotic cell comprises polynucleotides encoding the to be expressed pRS, ptRNA and POI, and further comprises, e.g. is fed with, said ncAA or salt thereof. Where expression of one of said pRS, ptRNA and/or POI is under the control of a TRE, the eukaryotic cell expediently further expresses a tetO-binding protein that is chosen to match the respective TRE used as described herein, and the eukaryotic cell is contacted with, e.g. cultured in a medium containing, tetracycline, doxycycline, or a functional analogue thereof as described herein.

Recombinant expression in eukaryotic cells, including the induction of TRE-controlled expression, is a standard tool in the art. The skilled person is therefore well able to choose conditions of culturing the eukaryotic cells so as to allow for the (recombinant) expression of expression products of interest (here: at least ptRNA, pRS and POI). Depending on the type of cell, a liquid medium can be used for culturing. Culture can be batchwise, semi-batchwise or continuous. Nutrients can be present at the beginning of the culturing or can be supplied later, semi-continuously or continuously.

For producing a POI (target polypeptide) according to a method of the present invention, the eukaryotic cell is cultured under suitable conditions, preferably in the presence of (e.g., in a culture medium containing) the ncAA or salt thereof, for a time suitable to allow translation at a ribosome of the cell. Depending on the polynucleotide(s) encoding the POI (and optionally the other elements pRS, ptRNA, etc.), it may be required to induce expression by adding a compound inducing transcription, such as, e.g., arabinose, isopropyl β-D-thiogalactoside (IPTG) or, in case of TRE-controlled expression, tetracycline, doxycycline, or a functional analog thereof. mRNA that encodes the POI (and comprises one or more than codon that is the reverse complement of the anticodon comprised by the ptRNA) is bound by the ribosome. Then, the POI is formed by stepwise attachment of amino acids and ncAAs at positions encoded by codons which are recognized (bound) by respective aminoacyl tRNAs. Thus, the ncAA(s) is/are incorporated in the POI at the position(s) encoded by the (selector) codon(s) that is/are the reverse complement of the anticodon comprised by the ptRNA.

The eukaryotic cells used for preparing a POI comprising one or more than one non-canonical amino acid (ncAA) residue as described herein can be prepared by introducing polynucleotides encoding the pRS, the ptRNA, the POI and/or further elements, such as a dsRNA-binding polypeptide and/or a tetO-binding polypeptide as described herein, into a eukaryotic (host) cell. Said nucleotide sequences can be located on separate nucleic acid molecules (vectors) or on the same nucleic acid molecule (e.g., vector), in any combination, and can be introduced into the cell in combination or sequentially.

Preferably common cloning and transfection techniques, known by a person skilled in the art, are used, for example co-precipitation, protoplast fusion, electroporation, virus-mediated gene delivery, lipofection, microinjection or others, for introducing the stated nucleic acid molecules in the respective cell. Suitable techniques are described for example in Current Protocols in Molecular Biology, F. Ausubel et al., Ed., Wiley Interscience, New York 1997, or Sambrook et al. Molecular Cloning: A Laboratory Manual. 2^{nd} edition, Cold Spring Harbor Laboratory, Cold Spring Harbor Laboratory Press. Cold Spring Harbor, NY, 1989.

After translation, the POI prepared according to the present invention may optionally be recovered from the eukaryotic cell or, if secreted, from the culture medium, and purified, either partially or substantially to homogeneity, according to procedures generally known in the art. For this purpose, the POI can be recovered and purified, either partially or substantially to homogeneity, according to procedures known to and used by those of skill in the art. Unless the target polypeptide is secreted into the culture medium, recovery usually requires cell disruption. Methods of cell disruption are well known in the art and include physical disruption. e.g., by (ultrasound) sonication, liquid-sheer disruption (e.g., via French press), mechanical methods (such as those utilizing blenders or grinders) or freeze-thaw cycling, as well as chemical lysis using agents which disrupt lipid-lipid, protein-protein and/or protein-lipid interactions (such as detergents), and combinations of physical disruption techniques and chemical lysis. Standard procedures for purifying polypeptides from cell lysates or culture media are also well known in the art and include, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. Antibodies made against the polypeptides of the invention can be used as purification reagents, i.e. for affinity-based purification of the polypeptides. A variety of purification/protein folding methods are well known in the art, including, e.g., those set forth in Scopes, Protein Purification, Springer, Berlin (1993); and Deutscher, Methods in Enzymology Vol. 182: Guide to Protein Purification, Academic Press (1990); and the references cited therein.

As noted, those of skill in the art will recognize that, after synthesis, expression and/or purification, polypeptides can possess a conformation different from the desired conformations of the relevant polypeptides. For example, overexpressed polypeptides as well as polypeptides produced by prokaryotic systems often are optimized by exposure to chaotropic agents to achieve proper folding. During purification from, e.g., lysates derived from E. coli, the expressed polypeptide is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Polypeptides can be refolded in a redox buffer containing, e.g., oxidized glutathione and L-arginine.

The POIs expressed by a method of the present invention can be purified by known techniques, such as, e.g., molecular sieve chromatography (gel filtration), such as Q-sepharose chromatography, ion exchange chromatography and hydrophobic chromatography, and other common protein purification techniques such as ultrafiltration, crystallization, salting-out, dialysis and native gel electrophoresis. Suitable methods are described, for example, in Cooper, T. G., Biochemische Arbeitsmethoden [Biochemical processes], Verlag Walter de Gruyter, Berlin, New York or in Scopes, R., Protein Purification, Springer Verlag, New York. Heidelberg. Berlin.

For isolating a POI, it can be advantageous to link the POI with a tag that can serve for easier purification. This can be achieved by introducing a corresponding tag-encoding sequence into the POI-encoding nucleotide sequence. Suitable tags for protein purification are well known in the art and include, e.g., histidine tags (e.g., His₆ tag) and epitopes that can be recognized as antigens of antibodies (described for example in Harlow, E. and Lane, D., 1988, Antibodies: A Laboratory Manual. Cold Spring Harbor (N.Y.) Press). These tags can serve for attaching the proteins to a solid carrier, for example a polymer matrix, which can for example be used as packing in a chromatography column, or can be used on a microtiter plate or on some other carrier.

A tag linked to a POI can also serve for detecting the POI. Tags for protein detection are well known in the art and include, e.g., fluorescent dyes, enzyme markers, which form a detectable reaction product after reaction with a substrate, and others.

Also described are POIs produced by the methods of the invention. Such POIs can be prepared by a method of the invention that makes use of a eukaryotic cell as described herein.

### 8. Kits

The present invention also provides kits for preparing a POI having one or more than one non-canonical amino acid (ncAA) residue in its amino acid sequence. The kit of the invention comprises one or more than one ncAA, or salt thereof, corresponding to the one or more than one ncAA residue of the POI. The kit may further comprise a polynucleotide, or combination of polynucleotides, of the present invention, and/or a polynucleotide encoding a dsRNA-binding polypeptide as described herein and/or an eukaryotic cell of the present invention. The kit may additionally comprise one or more reporter constructs encoding an easily detectable (e.g. fluorescent) reporter polypeptide. The report construct comprises at least one selector codon that is located in the encoding sequence of the report polypeptide such that successful incorporation of an amino acid or ncAA at the position encoded by said selector codon(s) allows for the translational synthesis of the reporter polypeptide.

The kits of the present invention can be used in methods of the invention for preparing ncAA-residue containing POIs as described herein.

### 9. Particular Embodiments

The present invention refers to the following particular embodiments:

1. According to a first embodiment the invention relates to an eukaryotic cell comprising:
(a) a polynucleotide encoding a prokaryotic aminoacyl tRNA synthetase (pRS),
(b) a polynucleotide encoding a prokaryotic tRNA (ptRNA) and one or more than one ribozyme,
wherein the pRS is capable of acylating the ptRNA, and
wherein the sequences encoding the ptRNA and the ribozyme(s) are linked such that transcription produces an RNA molecule comprising both the ptRNA and the ribozyme(s) (ptRNA-ribozyme).

A second embodiment relates to an eukaryotic cell of embodiment one, wherein the ribozyme is selected from a hammerhead ribozyme, a hairpin ribozyme, a Varkud satellite ribozyme, a glucosamine-6-phosphate synthetase riboswitch and a hepatitis delta virus (HDV) ribozyme.

A third embodiment relates to an eukaryotic cell of embodiment one or embodiment two, wherein the ribozyme is a HDV ribozyme which, in the ptRNA-ribozyme, is directly linked to the 3′ end of the ptRNA.

A fourth embodiment relates to an eukaryotic cell of any one of embodiments one to three, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein expression of said RNA polymerase is controlled by a TRE.

A fifth embodiment relates to an eukaryotic cell of any one of embodiments one to four, wherein the transcription of the pRS-encoding sequence is controlled by a tetracycline-responsive promoter element (TRE).

A sixth embodiment relates to an eukaryotic cell of embodiment four or embodiment five, further comprising a polynucleotide encoding a protein which binds to a tetracycline operator (tetO) sequence (tetO-binding protein), wherein the tetO-binding protein is selected from the group consisting of
(i) a tetracycline repressor (tetR).
(ii) a tetracycline-controlled transcriptional activator (tTA), and
(iii) a reverse tTA (rtTA).

A seventh embodiment relates to an eukaryotic cell of any one of embodiments one to six, wherein:
(i) transcriptional synthesis of the ptRNA-ribozyme is controlled by a T7 promoter, and/or
(ii) transcription of the pRS-encoding sequence is controlled by a T7 promoter,
and wherein the eukaryotic cell further comprises
(c) a polynucleotide encoding a T7 RNA polymerase (T7RNAP).

An eighth embodiment relates to an eukaryotic cell of embodiment seven, wherein expression of the T7RNAP is controlled by a TRE.

A ninth embodiment relates to an eukaryotic cell of embodiment seven or embodiment eight, wherein the T7RNAP carries
(i) a nuclear localization signal (NLS), or
(ii) a nuclear export signal (NES), or
(iii) none of NES and NLS.

A tenth embodiment relates to an eukaryotic cell of any one of embodiments one to nine further comprising a polynucleotide encoding a dsRNA-binding polypeptide as defined in any one of following embodiments eleven to thirteen.

An eleventh embodiment relates to an eukaryotic cell comprising:
(a) a polynucleotide encoding a prokaryotic aminoacyl tRNA synthetase (pRS),
(b) a polynucleotide encoding a prokaryotic tRNA (ptRNA), and
(c) a polynucleotide encoding a polypeptide that is capable of binding to double-stranded RNA (dsRNA-binding polypeptide) and comprises an amino acid sequence having at least 80%, 85%, 90%, 91%, 92%, 93%, 94%, 95%, 96%, 97%, 98%, or 99% identity to the sequence set forth in any one of SEQ ID NOs:37, and 57-59.

A twelfth embodiment relates to an eukaryotic cell of embodiment eleven, wherein the amino acid sequence comprised by the dsRNA-binding polypeptide has at least 80%, 85%, 90%, 91%, 92%, 93%, 94%, 95%, 96%, 97%, 98%, or 99% identity to the sequence set forth in SEQ ID NO:38.

A thirteenth embodiment relates to an eukaryotic cell of embodiment eleven or embodiment twelve, wherein the dsRNA-binding polypeptide is incapable of phosphorylating eukaryotic initiation factor 2α (eIF-2α).

A fourteenth embodiment relates to an eukaryotic cell of any one of embodiments eleven to thirteen, wherein:
(i) transcriptional synthesis of the ptRNA is controlled by a tetracycline-responsive promoter element (TRE), or
(ii) the ptRNA is transcriptionally synthesized by an RNA polymerase, wherein expression of said RNA polymerase is controlled by a TRE.

A fifteenth embodiment relates to an eukaryotic cell of any one of embodiments eleven to fourteen, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE.

A sixteenth embodiment relates to an eukaryotic cell of embodiment fourteen or embodiment fifteen, further comprising a polynucleotide encoding a protein which binds to a tetracycline operator (tetO) sequence (tetO-binding protein), wherein the tetO-binding protein is selected from the group consisting of:
(i) a tetracycline repressor (tetR),
(ii) a tetracycline-controlled transcriptional activator (tTA), and
(iii) a reverse tTA (rtTA).

A seventeenth embodiment relates to an eukaryotic cell of any one of embodiments eleven to sixteen, wherein:
(i) transcriptional synthesis of the ptRNA is controlled by a T7 promoter, or
(ii) transcription of the pRS-encoding sequence is controlled by a T7 promoter, or
(iii) both of (i) and (ii);
and wherein the eukaryotic cell further comprises
(c) a polynucleotide encoding a T7 RNA polymerase (T7RNAP).

An eighteenth embodiment relates to an eukaryotic cell of embodiment seventeen, wherein expression of the T7RNAP is controlled by a TRE.

A nineteenth embodiment relates to an eukaryotic cell of embodiment seventeen of embodiment eighteen, wherein the T7RNAP carries
(i) a nuclear localization signal (NLS), or
(ii) a nuclear export signal (NES), or
(iii) none of NES and NLS.

A twentieth embodiment relates to an eukaryotic cell of any one of embodiments eleven to thirteen, wherein the eukaryotic cell is a cell of any one of embodiments one to nine.

A twenty-first embodiment relates to a polynucleotide encoding an RNA molecule comprising a ptRNA and one or more than one ribozyme (ptRNA-ribozyme) as defined in any one of embodiments one to four and seven; wherein, more particularly,
a) the ribozyme is a HDV ribozyme which, in the ptRNA-ribozyme, is directly linked to the 3′ end of the ptRNA; or
b) the ptRNA is derived from a Methanosarcina species, in particular a pyrrolysyl tRNA of M. mazei; or
c) wherein a ptRNA molecule is encoded missing the 3′-terminal aminoacyl acceptor stem motif CCA; or
d) the ptRNA is encoded by the following nucleotide sequence (SEQ ID NO:60) ggaaacctgatcatgtagatcgaatggactxxxaatccgttcagccgggttagattcccggggtttccg wherein xxx encodes an anticodon;

A twenty-second embodiment relates to a polynucleotide, or combination of two or more polynucleotides, comprising a nucleotide sequence encoding an RNA molecule comprising a ptRNA and one or more than one ribozyme (ptRNA-ribozyme) as defined in any one of embodiments one to four and seven, and:
(i) a nucleotide sequence encoding a tetO-binding protein as defined in embodiment six, or
(ii) a nucleotide sequence encoding a pRS as defined in any one of embodiments one, five and seven, or
(ii) both of (i) and (ii).

A twenty-third embodiment relates to a method for preparing a polypeptide of interest (POI) having one or more than one non-canonical amino acid (ncAA) residue in its amino acid sequence, wherein the method comprises:
(a) expressing, in a eukaryotic cell:
   a prokaryotic aminoacyl tRNA synthetase (pRS) and
   an RNA molecule comprising a prokaryotic tRNA (ptRNA) and one or more than one ribozyme (ptRNA-ribozyme);
and concomitantly or sequentially
(b) expressing the POI in the eukaryotic cell in the presence of one or more than one ncAA or salt thereof corresponding to the one or more than one ncAA residue of the POI; and
(c) optionally recovering the expressed POI;
wherein the pRS is capable of acylating the ptRNA with the ncAA or the salt thereof,
wherein the POI is encoded by a nucleotide sequence that comprises one or more than one selector codon encoding the one or more than one ncAA residue, and
wherein the selector codon is the reverse complement of the anticodon of the ptRNA.

A twenty-fourth embodiment relates to a method of embodiment twenty-three, wherein the ribozyme is selected from a hammerhead ribozyme, a hairpin ribozyme, a Varkud satellite ribozyme, a glucosamine-6-phosphate synthetase riboswitch and a hepatitis delta virus (HDV) ribozyme.

A twenty-fifth embodiment relates to a method of embodiment twenty-three or embodiment twenty-four, wherein the ribozyme is a HDV ribozyme which, in the ptRNA-ribozyme, is directly linked to the 3′ end of the ptRNA.

### T7-Controlled in General

A twenty-sixth embodiment relates to a method of any one of embodiments twenty-three to twenty-five, wherein the transcriptional synthesis of the ptRNA-ribozyme is controlled by a T7 promoter, and wherein the method further comprises expressing a T7 RNA polymerase (T7RNAP) in the eukaryotic cell prior to or concomitantly with the thereby controlled expression of the ptRNA-ribozyme (i) or the pRS (ii) or both (iii), respectively.

T-REx System (tetR, +tet/dox)

A twenty-seventh embodiment relates to a method of any one of embodiments twenty-three to twenty-five, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein the expression of said RNA polymerase is controlled by a TRE;
wherein the method further comprises expressing a tetracycline repressor (tetR) in the eukaryotic cell at least prior to the expression of the ptRNA-ribozyme, and
wherein expression of the ptRNA-ribozyme involves contacting the eukaryotic cell with tetracycline, doxycycline, or a functional analogue thereof, thereby inducing transcriptional synthesis of the ptRNA-ribozyme.

A twenty-eighth embodiment relates to a method of any one of embodiments twenty-three to twenty-five and twenty-seven, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a tetracycline repressor (tetR) in the eukaryotic cell at least prior to the expression of the pRS; and
wherein expression of the pRS involves contacting the eukaryotic cell with tetracycline, doxycycline, or afunctional analogue thereof, thereby inducing transcription of the pRS-encoding sequence.

A twenty-ninth embodiment relates to a method of embodiment twenty-seven or embodiment twenty-eight, wherein:
(i) transcriptional synthesis of the ptRNA-ribozyme is controlled by a T7 promoter, or
(ii) transcription of the pRS-encoding sequence is controlled by a T7 promoter, or
(iii) both of (i) and (ii);
and wherein the method further comprises:
   expressing a T7 RNA polymerase (T7RNAP) in the eukaryotic cell prior to or concomitantly with the thereby controlled expression of the ptRNA-ribozyme (i) or the pRS (ii) or both (iii), respectively, wherein expression of the T7RNAP is controlled by a TRE, and
   expressing a tetR in the eukaryotic cell at least concomitantly with the expression of the T7RNAP;
wherein expression of the T7RNAP involves contacting the eukaryotic cell with tetracycline, doxycycline, or a functional analogue thereof, thereby inducing transcription of the T7RNAP-encoding sequence.
   Tet-Off System (TA, −tet/dox)

A thirtieth embodiment relates to a method of any one of embodiments twenty-three to twenty-five, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein expression of said RNA polymerase is controlled by a TRE;
wherein the method further comprises:
(1) expressing a tetracycline-controlled transcriptional activator (tTA) in the eukaryotic cell prior to and concomitantly with the expression of the ptRNA-ribozyme, and
(2) keeping the eukaryotic cell prior to the expression of the ptRNA-ribozyme in contact with tetracycline, doxycycline, or a functional analogue thereof, thereby suppressing the transcriptional synthesis of the ptRNA-ribozyme, and then reducing the concentration of, or preferably removing, said tetracycline, doxycycline, or functional analogue thereof so as to induce transcriptional synthesis of the ptRNA-ribozyme.

A thirty-first embodiment relates to a method of any one of embodiments twenty-three to twenty-five and thirty, wherein:
(i) transcription of the pRS-encoding sequence is controlled by a TRE, or
(ii) transcription of the pRS-encoding sequence is catalyzed by an RNA polymerase, wherein the expression of said RNA polymerase is controlled by a TRE;
wherein the method further comprises:
(1) expressing a tetracycline-controlled transcriptional activator (tTA) in the eukaryotic cell prior to and concomitantly with the expression of the pRS, and
(2) keeping the eukaryotic cell prior to the expression of the pRS in contact with tetracycline, doxycycline, or a functional analogue thereof, thereby suppressing transcription of the pRS-encoding sequence, and then reducing the concentration of, or preferably removing, said tetracycline, doxycycline, or functional analogue thereof so as to induce transcription of the pRS-encoding sequence.

A thirty-second embodiment relates to a method of embodiment thirty or embodiment thirty-one, wherein:
(i) transcriptional synthesis of the ptRNA-ribozyme is controlled by a T7 promoter, or
(ii) transcription of the pRS-encoding sequence is controlled by a T7 promoter, or
(iii) both of (i) and (ii);
wherein the method further comprises:
(3) expressing a T7 RNA polymerase (T7RNAP) in the eukaryotic cell prior to or concomitantly with the thereby controlled expression of the ptRNA-ribozyme (i) or the pRS (ii) or both (iii), respectively,
   wherein expression of the T7RNAP is controlled by a TRE,
(4) expressing a tTA in the eukaryotic cell prior to and concomitantly with the expression of the T7RNAP, and
(5) keeping the eukaryotic cell prior to the expression of the T7RNAP in contact with tetracycline, doxycycline, or a functional analogue thereof, thereby suppressing transcription of the T7RNAP-encoding sequence, and then reducing the concentration of, or preferably removing, said tetracycline, doxycycline, or functional analogue thereof so as to induce transcription of the T7RNAP-encoding sequence.
   Tet-On system (rtTA, +dox)

A thirty-third embodiment relates to a method of any one of embodiments twenty-three to twenty-five, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein the expression of said RNA polymerase is controlled by a TRE;
wherein the method further comprises expressing a reverse tTA (rtTA) in the eukaryotic cell at least concomitantly with the expression of the ptRNA, and
wherein expression of the ptRNA-ribozyme involves contacting the eukaryotic cell with doxycycline, or a functional analogue thereof, thereby inducing transcriptional synthesis of the ptRNA-ribozyme.

A thirty-fourth embodiment relates to a method of any one of embodiments twenty-three to twenty-five and thirty-three, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a reverse tTA (rtTA) in the eukaryotic cell at least concomitantly with the expression of the pRS; and
wherein expression of the pRS involves contacting the eukaryotic cell with doxycycline, or a functional analogue thereof, thereby inducing transcription of the pRS-encoding sequence.

A thirty-fifth embodiment relates to a method of embodiment thirty-three or embodiment thirty-four, wherein:
(i) transcriptional synthesis of the ptRNA-ribozyme is controlled by a T7 promoter, or
(ii) transcription of the pRS-encoding sequence is controlled by a T7 promoter, or
(iii) both of (i) and (ii);
and wherein the method further comprises:
   expressing a T7 RNA polymerase (17RNAP) in the eukaryotic cell prior to or concomitantly with the thereby controlled expression of the ptRNA-ribozyme (i) or the pRS (ii) or both (iii), respectively, wherein expression of the T7RNAP is controlled by a TRE, and
   expressing a rtTA in the eukaryotic cell at least concomitantly with the expression of the T7RNAP;
wherein expression of the T7RNAP involves contacting the eukaryotic cell with doxycycline, or a functional analogue thereof, thereby inducing transcription of the T7RNAP-encoding sequence.

A thirty-sixth embodiment relates to a method of any one of embodiments twenty-three to twenty-five further comprising:
expressing, in the eukaryotic cell, a dsRNA-binding polypeptide as defined in any one of embodiments eleven to thirteen.

A thirty-seventh embodiment relates to a method for preparing a polypeptide of interest (POI) having one or more than one non-canonical amino acid (ncAA) residue in its amino acid sequence, wherein the method comprises:
(a) expressing, in a eukaryotic cell:
   a prokaryotic aminoacyl tRNA synthetase (pRS).
   a prokaryotic tRNA (ptRNA) and
   a polypeptide capable of binding to double-stranded RNA (dsRNA-binding polypeptide);
   and concomitantly or sequentially
(b) expressing the POI in the eukaryotic cell in the presence of one or more than one ncAA or salt thereof corresponding to the one or more than one ncAA residue of the POI; and
(c) optionally recovering the expressed POI;
wherein the pRS is capable of acylating the ptRNA with the ncAA or the salt thereof,
wherein the dsRNA-binding polypeptide comprises an amino acid sequence having at least 80%, 85%, 90%, 91%, 92%, 93%, 94%, 95%, 96%, 97%, 98%, or 99% identity to the sequence set forth in any one of SEQ ID NOs:37, and 57-59;
wherein the POI is encoded by a nucleotide sequence that comprises one or more than one selector codon encoding the one or more than one ncAA residue, and
wherein the selector codon is the reverse complement of the anticodon of the ptRNA.

A thirty-eighth embodiment relates to a method of embodiment thirty-seven, wherein the amino acid sequence comprised by the dsRNA-binding polypeptide has at least 80%, 85%, 90%, 91%, 92%, 93%, 94%, 95%, 96%, 97%, 98%, or 99% identity to the sequence set forth in SEQ ID NO:38.

A thirty-ninth embodiment relates to a method of embodiment thirty-seven or embodiment thirty-eight, wherein the dsRNA-binding polypeptide is incapable of phosphorylating eukaryotic initiation factor 2α (eIF-2α).

### T7-Controlled in General

A fortieth embodiment relates to a method of any one of embodiments thirty-seven to thirty-nine, wherein:
(i) transcriptional synthesis of the ptRNA is controlled by a T7 promoter, or
(ii) transcription of the pRS-encoding sequence is controlled by a T7 promoter, or
(iii) both of (i) and (ii);
wherein the method further comprises expressing a T7 RNA polymerase (T7RNAP) in the eukaryotic cell prior to or concomitantly with the thereby controlled expression of the ptRNA (i) or the pRS (ii) or both (iii), respectively.
   T-REx System (tetR, +tet/dox)

A forty-first embodiment relates to a method of any one of embodiments thirty-seven to thirty-nine, wherein:
(i) transcriptional synthesis of the ptRNA is controlled by a tetracycline-responsive promoter element (TRE), or
(ii) the ptRNA is transcriptionally synthesized by an RNA polymerase, wherein the expression of said RNA polymerase is controlled by a TRE;
wherein the method further comprises expressing a tetracycline repressor (tetR) in the eukaryotic cell at least prior to the expression of the ptRNA, and
wherein expression of the ptRNA involves contacting the eukaryotic cell with tetracycline, doxycycline, or a functional analogue thereof, thereby inducing transcriptional synthesis of the ptRNA.

A forty-second embodiment relates to a method of any one of embodiments thirty-seven to thirty-nine and forty-one, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a tetracycline repressor (tetR) in the eukaryotic cell at least prior to the expression of the pRS; and
wherein expression of the pRS involves contacting the eukaryotic cell with tetracycline, doxycycline, or a functional analogue thereof, thereby inducing transcription of the pRS-encoding sequence.

A forty-third embodiment relates to a method of embodiment forty-one or embodiment forty-two, wherein:
(i) transcriptional synthesis of the ptRNA is controlled by a T7 promoter, or
(ii) transcription of the pRS-encoding sequence is controlled by a T7 promoter, or
(iii) both of (i) and (ii);
and wherein the method further comprises:
   expressing a T7 RNA polymerase (T7RNAP) in the eukaryotic cell prior to or concomitantly with the thereby controlled expression of the ptRNA (i) or the pRS (ii) or both (iii), respectively, wherein expression of the T7RNAP is controlled by a TRE, and
   expressing a tetR in the eukaryotic cell at least concomitantly with the expression of the T7RNAP;
wherein expression of the T7RNAP involves contacting the eukaryotic cell with tetracycline, doxycycline, or a functional analogue thereof, thereby inducing transcription of the T7RNAP-encoding sequence.
   Tet-Off System (tTA, −tet/dox)

A forty-fourth embodiment relates to a method of any one of embodiments thirty-seven to thirty-nine, wherein:
(i) transcriptional synthesis of the ptRNA is controlled by a tetracycline-responsive promoter element (TRE), or
(ii) the ptRNA is transcriptionally synthesized by an RNA polymerase, wherein expression of said RNA polymerase is controlled by a TRE;
wherein the method further comprises:
(1) expressing a tetracycline-controlled transcriptional activator (tTA) in the eukaryotic cell prior to and concomitantly with the expression of the ptRNA, and
(2) keeping the eukaryotic cell prior to the expression of the ptRNA in contact with tetracycline, doxycycline, or a functional analogue thereof, thereby suppressing the transcriptional synthesis of the ptRNA, and then reducing the concentration of, or preferably removing, said tetracycline, doxycycline, or functional analogue thereof so as to induce transcriptional synthesis of the ptRNA.

A forty-fifth embodiment relates to a method of any one of embodiments thirty-seven to thirty-nine and forty-four, wherein:
(i) transcription of the pRS-encoding sequence is controlled by a TRE, or
(ii) transcription of the pRS-encoding sequence is catalyzed by an RNA polymerase, wherein the expression of said RNA polymerase is controlled by a TRE;
wherein the method further comprises:
(1) expressing a tetracycline-controlled transcriptional activator (tTA) in the eukaryotic cell prior to and concomitantly with the expression of the pRS, and
(2) keeping the eukaryotic cell prior to the expression of the pRS in contact with tetracycline, doxycycline, or a functional analogue thereof, thereby suppressing transcription of the pRS-encoding sequence, and then reducing the concentration of, or preferably removing, said tetracycline, doxycycline, or functional analogue thereof so as to induce transcription of the pRS-encoding sequence.

A forty-sixth embodiment relates to a method of embodiment forty-four or embodiment forty-five, wherein:
(i) transcriptional synthesis of the ptRNA is controlled by a T7 promoter, or
(ii) transcription of the pRS-encoding sequence is controlled by a T7 promoter, or
(iii) both of (i) and (ii);
wherein the method further comprises:
(3) expressing a T7 RNA polymerase (T7RNAP) in the eukaryotic cell prior to or concomitantly with the thereby controlled expression of the ptRNA (i) or the pRS (ii) or both (iii), respectively,
   wherein expression of the T7RNAP is controlled by a TRE,
(4) expressing a tTA in the eukaryotic cell prior to and concomitantly with the expression of the T7RNAP, and
(5) keeping the eukaryotic cell prior to the expression of the T7RNAP in contact with tetracycline, doxycycline, or afunctional analogue thereof, thereby suppressing transcription of the T7RNAP-encoding sequence, and then reducing the concentration of, or preferably removing, said tetracycline, doxycycline, or functional analogue thereof so as to induce transcription of the T7RNAP-encoding sequence.
   Tet-On System (rtTA, +dox)

A forty-seventh embodiment of a method of any one of embodiments thirty-seven to thirty-nine, wherein:
(i) transcriptional synthesis of the ptRNA is controlled by a tetracycline-responsive promoter element (TRE), or
(ii) the ptRNA is transcriptionally synthesized by an RNA polymerase, wherein the expression of said RNA polymerase is controlled by a TRE;
wherein the method further comprises expressing a reverse tTA (rtTA) in the eukaryotic cell at least concomitantly with the expression of the ptRNA, and
wherein expression of the ptRNA involves contacting the eukaryotic cell with doxycycline, or a functional analogue thereof, thereby inducing transcriptional synthesis of the ptRNA.

A forty-eighth embodiment of a method of any one of embodiments thirty-seven to thirty-nine and forty-seven, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a reverse tTA (rtTA) in the eukaryotic cell at least concomitantly with the expression of the pRS; and
wherein expression of the pRS involves contacting the eukaryotic cell with doxycycline, or a functional analogue thereof, thereby inducing transcription of the pRS-encoding sequence.

A forty-ninth embodiment relates to a method of embodiment forty-seven or embodiment forty-eight, wherein:
(i) transcriptional synthesis of the ptRNA is controlled by a T7 promoter, or
(ii) transcription of the pRS-encoding sequence is controlled by a T7 promoter, or
(iii) both of (i) and (ii);
and wherein the method further comprises:
   expressing a T7 RNA polymerase (T7RNAP) in the eukaryotic cell prior to or concomitantly with the thereby controlled expression of the ptRNA (i) or the pRS (ii) or both (iii), respectively, wherein expression of the T7RNAP is controlled by a TRE, and
   expressing a rtTA in the eukaryotic cell at least concomitantly with the expression of the T7RNAP;
wherein expression of the T7RNAP involves contacting the eukaryotic cell with doxycycline, or a functional analogue thereof, thereby inducing transcription of the T7RNAP-encoding sequence.

A fiftieth embodiment relates to a method of any one of embodiments thirty-seven to forty-nine, wherein the ptRNA is expressed a part of an RNA molecule further comprising one or more than one ribozyme (ptRNA-ribozyme).

A fifty-first embodiment relates to a method of embodiment fifty, wherein the method is a method of any one of embodiments twenty-three to thirty-five.

A fifty-second embodiment relates to a kit for preparing a polypeptide of interest (POI) having one or more than one non-canonical amino acid (ncAA) residue in its amino acid sequence, wherein the kit comprises one or more than one ncAA, or salt thereof, corresponding to the one or more than one ncAA residue of the POI, and:
(a) a polynucleotide of embodiment twenty-one, or
(b) a polynucleotide, or combination of two or more polynucleotides, of embodiment twenty-two, or
(c) a polynucleotide encoding a dsRNA-binding polypeptide as defined in any one of embodiments eleven to thirteen, or
(d) an eukaryotic cell of any one of embodiments one to twenty.

The invention is now explained in more detail by making reference to the particular, non-limiting embodiments of the subsequent experiment section.

### EXAMPLES

### Material and Methods

Unless stated otherwise, the cloning and expression of recombinant polypeptides was carried out by standard methods, as described for example in Sambrook, J., Fritsch, E. F. and Maniatis, T., Molecular Cloning: A Laboratory Manual, 2^{nd} edition, Cold Spring Harbor Laboratory. Cold Spring Harbor Laboratory Press, Cold Spring Harbor, NY, 1989.

### A) Cloning

The reporter gene NLS-iRFP-Flag-GFP^{Y39->TAG}-6His (SEQ ID NO:39) was cloned into a pCI plasmid (Promega) as described in Nikic et al. (Angew Chem Int Ed Engl 2016, 55:16172-16176). NLS-iRFP-Flag-GFP^{Y39->TAG}-6His contains iRFP fused to a GFP wherein position 39 of the GFP is encoded by the amber stop codon TAG (iRFP-GFP^{Y39TAG}).

The coding sequence of T7 RNA polymerase gene with and without a NES signal (NES-T7RNAP or T7RNAP) was inserted into the pcDNA3.1 plasmid or pCAGGS plasmid. The pcDNA3.1 plasmid and pCAGGS plasmid differ in the promoter the pcDNA3.1 plasmid comprises the CMV promoter; the pCAGGS plasmid comprises a CMV enhancer with a chicken actin promoter and an intron (SEQ ID NO: 56). For constitutive expression the T7RNAP- or NES-T7RNAP-encoding sequence was inserted downstream of the CMV promoter (pcDNA3.1 plasmid) or downstream of the chicken actin promoter and intron (pCAGGS plasmid). For inducible expression via T-REx system, the NES-T7RNAP-encoding sequence was inserted downstream of a “T-REx” promoter comprising two tetO sequences (SEQ ID NO:12, ThermoFisher Scientific). For inducible expression via Tet-On system, the NES-T7RNAP-encoding sequence was inserted downstream of a “Tet-On” promoter comprising 8 tetO sequences (SEQ ID NO:10). The expression cassettes for constitutive, “T-REx” and “Tet-On” expression of NES-T7RNAP are set forth in SEQ ID NOs:40-43, respectively.

For constitutive expression the coding sequence for PylRS^{AF} (from Methanosarcina mazei) with or without a NES signal (NES-PylRS^{AF} or PylRS^{AF}) was inserted into a pcDNA3.1 plasmid (Invitrogen) downstream of the CMV promoter. For inducible expression via T-REx system, the NES-PylRS^{AF}-encoding sequence was inserted into the plasmid pDEST downstream of a “T-REx” promoter comprising two tetO sequences (SEQ ID NO:12, ThermoFisher Scientific). For inducible expression via Tet-On system, the NES-PylRS^{AF} encoding sequence was inserted into the pcDNA3.1 plasmid downstream of a “Tet-On” promoter comprising 8 tetO sequences (SEQ ID NO:10). The expression cassettes for constitutive, “T-REx”- and “Tet-On”-expression of NES-PylRS^{AF} are set forth in SEQ ID NO:44-47, respectively.

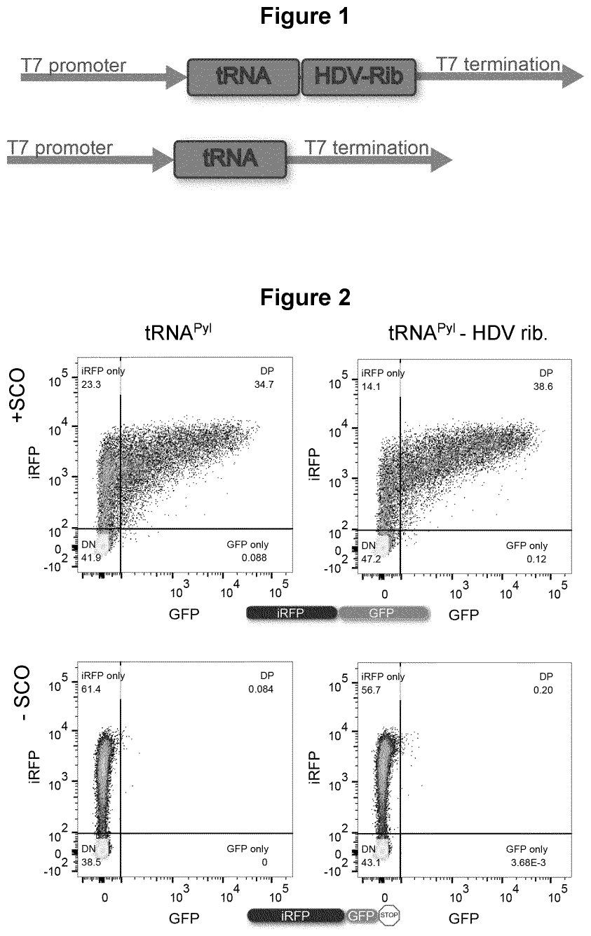

A T7 promoter-controlled expression cassette for tRNA^{Pyl} (SEQ ID NO:48) was generated by cloning a T7 promoter sequence upstream of the tRNA^{Pyl} encoding sequence, and a T7 termination signal downstream of the tRNA^{Pyl} encoding sequence, and insertion of this expression cassette into a p2RZ plasmid (Avis et al., Methods Mol Biol. 941:83-98, 2012; (see FIG. 1)). A variant of this expression cassette having a HDV ribozyme (tRNA^{Pyl}-HDV) was generated by a combination of restriction cloning, site-directed mutagenesis and restriction-free cloning so as to insert the HDV ribozyme encoding sequence directly downstream of the tRNA^{Pyl} (SEQ ID NO:49, cf. FIG. 1). For inducible expression via T-REx system, two different expression cassettes under the control of tet-inducible promoters were generated by inserting the tRNA^{Pyl}-encoding sequence into the plasmids pUC57 (in case of the U6 promoter) and pPBEX (in case of the H1 promoter) downstream of a variant of either a human U6 promoter comprising eight tetO sequences before the U6 [promoter and two tetO sequences after or a human H1 promoter comprising two tetO sequences (SEQ ID NOs:50 and 51).

Expression cassettes for an rtTA or a tetR contained the rtTA-encoding sequence (SEQ ID NO:14) downstream of the CMV promoter and the tetR-encoding sequence (SEQ ID NO:13) downstream of the CMV promoter.

The N-terminal dsRNA-binding domain (residues 2-174) of human protein kinase R (“PKR(2-174)”) was cloned into a p1.18 plasmid (U.S. Pat. No. 6,187,759 B1, pUC based plasmid containing a hCMV promoter, intron A, a terminator sequence and a multiple cloning site) for constitutive expression together with an N-terminal HA-tag under the control of the CMV promoter fused to intron A (the biggest intron of the transcribed region of the hCMV major immediate-early (IE1) gene, which enhances expression of protein. The cytomegalovirus (hCMV) is also called human beta-herpes virus 5 (HHV-5); cf. Chapman, B. S. et al NAR, 1991, Vol. 19, N0 14, 2979-3986). The amino acid sequence of HA-PKR(2-174) is set forth in SEQ ID NO:36.

### B) Cell Culture and Transfection

HEK293T cells (ATCC CRL-3216) and HEK Flp-In T-REx 293 cells (ThermoFisher Scientific, #R78007) were maintained in Dulbecco's modified Eagle's medium (Life Technologies #41965-039) supplemented with 1% penicillin-streptomycin (Sigma P0781, 10,000 U/ml penicillin, 10 mg/ml streptomycin, 0.9% NaCl), 1% L-glutamine (Sigma #G7513), 1% sodium pyruvate (Life Technologies #11360), and 10% Fetal Bovine Serum (FBS, Sigma #F7524). For HEK Flp-In T-REx 293 cells the medium was supplemented with blasticidin (15 μg/ml) accordingly to the manufacturer's manual. The HEK Flp-In T-REx 293 cell line is derived from HEK293 cells (ATCC CRL-1573) and stably expresses the tet repressor (tetR) gene under the control of the constitutive CMV promoter. See user guide “Growth and Maintenance of the Flp-In™ T-REx™ Cell Line”, catalog no. R780-07, document part no. 25-0369, publication no. MAN0000187, revision 2.0, 14 Sep. 2015 by Gibco/Life Technologies.

Cells were cultured at 37° C. in a 5% CO₂ atmosphere and passaged every 2-3 days up to 15-20 passages.

Cells were seeded 15-20 hours prior to transfection (˜110.000 cells/well) and grown in 24-well plate cell culture plates at 37° C. in a 5% CO₂ atmosphere to a confluency of 70-80%. For HEK TetOn-3G cells the cell culture plates had previously been coated with polylysine hydrobromide for 4-10 h. For three and four plasmid transfections 1.2 μg total DNA/well was used. For five or six plasmid transfections 1.5 μg total DNA/well was used. Transfections were done with polyethylenimine (PEI) at a DNA to PEI ratio of 1 to 3.

C) Induction of Expression and Feeding with ncAAs

Four hours after transfection the medium was exchanged for fresh medium. Depending on the experiment and sample, said fresh medium contained 1 μg/ml tetracycline (for induction of expression in T-REx systems, systems with tetR co-expression) or 1 μg/ml doxycycline (for induction of expression in Tet-On systems) and/or 250 μM of the used ncAA (e.g., BOC), or none of these (indicated concentrations=final concentration in the cell culture).

The cells were then incubated at 37° C. in 5% CO₂ atmosphere for 2 days and afterwards analyzed as described herein.

### D) Analysis by Flow Cytometry Spectroscopy (FCS)

Cells were washed once with 1×PBS and analyzed via flow cytometry spectroscopy. Data acquisition and analysis were performed using a BD LSRFortessa instrument (BD Bioscience) and the FlowJo software (FlowJo). GFP fluorescence was acquired using the 488 nm laser with detection at 530/30 nm, and iRFP fluorescence was acquired using 670 nm laser with detection at 730145 nm. The measured events were gated first in the FSC-A×SSC-A scatter plot for live cells, and then in the FSC-A×SSC-W scatter plot for singlets. In the figures, the events of the singlet gate are shown as a plot of GFP signal vs iRFP signal, which is divided into four areas showing four different species: cell with iRFP fluorescence (lacking GFP) in the top left, cells with both iRFP and GFP fluorescence (expressing the full-length protein iRFP-GFP, called “double positive specimen” or “DPs”) in the top right, cells with GFP fluorescence (lacking iRFP) in the lower right, and cells lacking both iRFP and GFP in the lower left.

The reporter NLS-iRFP-Flag-GFP^{Y39->TAG}-6His is a fusion of iRFP (infrared fluorescent protein) and GFP (green fluorescent protein) wherein amino acid position 39 of the GFP is encoded by the amber (TAG) stop codon. Cells express iRFP (detectable as red fluorescence) if properly transfected and further express GFP (detectable as green fluorescence) only if the amber codon is suppressed to encode the ncAA (here: BOC or SCO). The ratio of green fluorescence (GFP) to red fluorescence (iRFP) in the cells is therefore an indicator of amber suppression efficiency.

### Example 1: Amber Suppression Using tRNA^{Pyl} Expressed as RNA-Ribozyme

Amber suppression was examined using the reporter gene NLS-iRFP-Flag-GFP^{Y39->TAG}-6His, PylRS^{AF}, tRNA^{Pyl} and the ncAA SCO in the presence of PKR(2-174). PKR(2-174) was expressed together with an N-terminal HA-tag (SEQ ID NO:36) as HA-PKR(2-174). tRNA^{Pyl} was expressed together with a HDV ribozyme as a tRNA-ribozyme RNA molecule (tRNA^{Pyl}-HDV). PylRS^{AF} was expressed under the control of a CMV promoter. The cells further expressed T7 RNA polymerase (T7RNAP).

PylRS^{AF} and T7RNAP were constitutively expressed using HEK293T cells (ATCC CRL-3216).

HEK293T cells were transfected with vectors carrying expression cassettes for the reporter gene NLS-iRFP-Flag-GFP^{Y39->TAG}-6His, PylRS^{AF} under the control of the CMV promoter, tRNA^{Pyl}-HDV under the control of a T7 promoter and T7RNAP under the control of the CMV promoter using the respective constructs and transfection method described in sections A) and B) above.

The controls were prepared identically with the exception that cells were not fed with SCO (“−ncAA”) and/or the tRNA^{Pyl} was expressed without the HDV ribozyme.

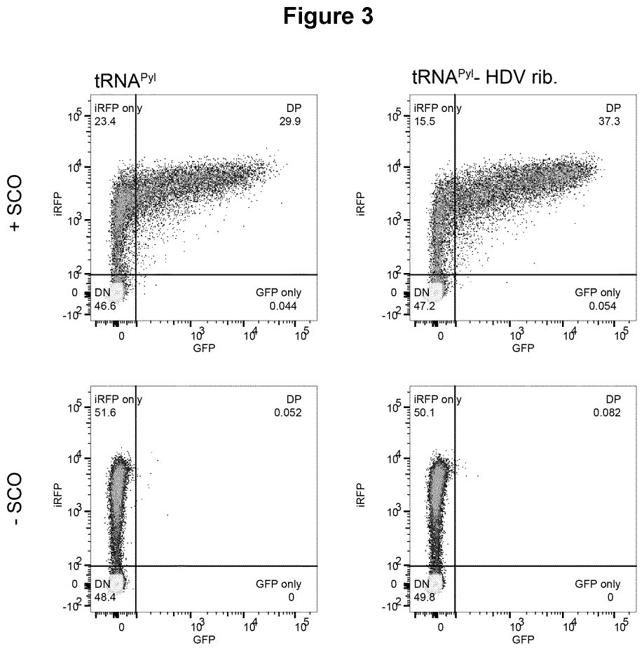

The cells were analyzed by FCS as described in section D) above. The results shown in FIG. 2 (using the pcDNA3.1 plasmid) and FIG. 3 (using the pCAGGS plasmid) indicate that expression of tRNA^{Pyl} together with HDV ribozyme resulted in more efficient amber suppression compared to tRNA^{Pyl} expressed without a ribozyme. Without wishing to be bound by theory it is assumed that this effect results from the ribozyme improving tRNA processing.

### Example 2: Amber Suppression Using Different Inducible Expression Systems

Amber suppression was examined using the reporter gene NLS-iRFP-Flag-GFP^{Y39->TAG}-6His, PylRS^{AF}, tRNA^{Pyl} und the ncAA BOC. tRNA^{Pyl} was expressed together with a HDV ribozyme as a tRNA-ribozyme RNA molecule (tRNA^{Pyl}-HDV) under the control of a T7 promoter. PylRS^{AF} was expressed together with a NES signal (NES-PylRS^{AF}). The cells further expressed T7 RNA polymerase together with a NES signal (NES-T7RNAP).

NES-PylRS^{AF} and NES-T7RNAP were inducibly expressed using either the T-REx system or the Tet-On system.

For the T-REx system. HEK Flp-In T-REx 293 cells were transfected with vectors carrying expression cassettes for the reporter gene NLS-iRFP-Flag-GFP^{Y39->TAG}-6His, NES-PylRS^{AF} under the control of a “T-REx” promoter, tRNA^{Pyl}-HDV under the control of a T7 promoter and NES-T7RNAP under the control of a “T-REx” promoter using the respective constructs and transfection method described in sections A) and B) above. HEK Flp-In T-REx 293 cells stably express the tet repressor (tetR) gene. Expression of NES-T7RNAP (and thereby tRNA^{Pyl}-HDV) and NES-PylRS^{AF} was induced by adding tetracycline and the cells were fed with the ncAA BOC as described in section C) above.

For the Tet-On system, HEK293T cells (ATCC CRL-3216) were co-transfected with vectors carrying expression cassettes for the reporter gene NLS-iRFP-Flag-GFP^{Y39->TAG}-6His, NES-PylRS^{AF} under the control of a “Tet-On” promoter, tRNA^{Pyl}-HDV under the control of a T7 promoter, NES-T7RNAP under the control of a “Tet-On” promoter and rtTA using the respective constructs and transfection method described in sections A) and B) above in the presence of PKR(2-174). PKR(2-174) was expressed together with an N-terminal HA-tag (SEQ ID NO:36) as HA-PKR(2-174). Expression of NES-T7RNAP (and thereby tRNA^{Pyl}-HDV) and NES-PylRS^{AF} was induced by adding doxycycline and the cells were fed with the ncAA BOC as described in section C) above.

The controls were prepared identically with the exception that neither tetracycline nor doxycycline was added.

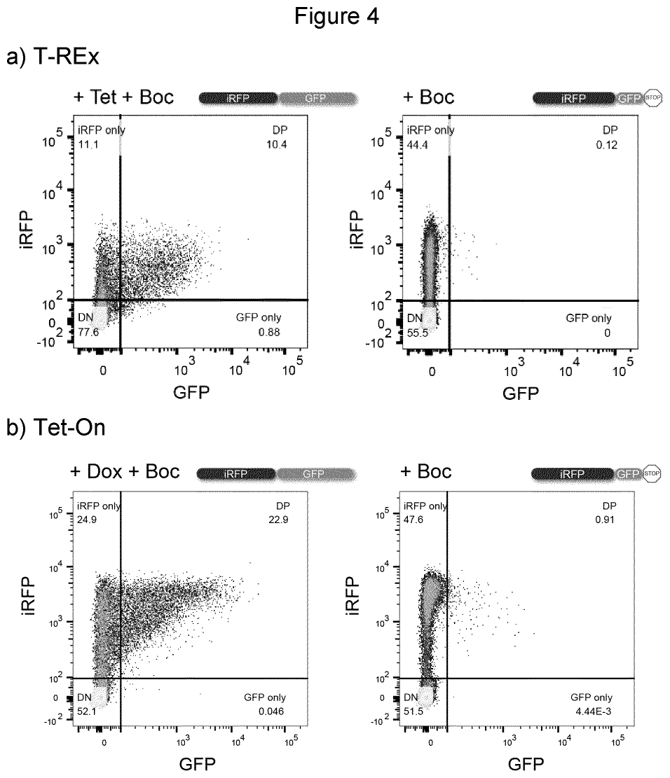

The cells were analyzed by FCS as described in section D) above. The results shown in FIG. 4 indicate that amber suppression using BOC together with inducibly expressed tRNA^{Pyl}-HDV and NES-PylRS^{AF} worked effectively in both the T-REx system (FIG. 4a) and the Tet-On system (FIG. 4b).

### Example 3: Effect of PKR(2-174) on Amber Suppression in Inducible and Non-Inducible Expression Systems

Amber suppression was examined using the reporter gene NLS-iRFP-Flag-GFP^{Y39->TAG}-6His, PylRS^{AF}, tRNA^{Pyl}, tetR und the ncAA BOC in the presence or absence of PKR(2-174).

PKR(2-174) was expressed together with an N-terminal TA-tag (SEQ ID NO:36) as HA-PKR(2-174).

One of three different expression cassettes for tRNA^{Pyl} was used: T7-tRNA^{Pyl}-HDV wherein tRNA^{Pyl} was expressed together with a HDV ribozyme (located at the 3′-end of tRNA^{Pyl}) as a tRNA-ribozyme RNA molecule (tRNA^{Pyl}-HDV) under the control of a T7 promoter; 8×tetO-U6-tetO-tRNA^{Pyl} wherein expression of tRNA^{Pyl} is controlled by a tetracycline-inducible variant of the human U6 promoter comprising eight tetO sequences before the U6 promoter and two tetO sequences after the U6 promoter (SEQ ID NO:50); and H1-TetO-tRNA^{Pyl} wherein expression of tRNA^{Pyl} is controlled by a tetracycline-inducible variant of the human H1 promoter comprising two tetO sequences (SEQ ID NO:51).

PylRS^{AF} was expressed together with a NES signal (NES-PylRS^{AF}). One of two different expression cassettes for NES-PylRS^{AF} was used: CMV-tetO-NES-PylRS^{AF} wherein expression of NES-PylRS^{AF} is controlled by a tetracycline-inducible variant of the CMV promoter comprising two tetO sequences (SEQ ID NO:46); CMV-NES-PylRS^{AF} wherein expression of NES-PylRS^{AF} is controlled by the (constitutive) CMV promoter (SEQ ID NO:45).

Cells wherein expression of tRNA^{Pyl} was controlled by a T7 promoter (expression cassette T7-tRNA^{Pyl}-HDV) further contained one of two different expression cassettes for T7 RNA polymerase to be expressed together with a NES signal (NES-T7RNAP); CMV-tetO-T7RNAP wherein NES-T7RNAP expression is under the control of a tetracycline-inducible variant of the CMV promoter comprising two tetO sequences (SEQ ID NO:12); CMV-NES-T7RNAP wherein NES-T7RNAP expression is under the control of the (constitutive) CMV promoter (SEQ ID NO:43).

HEK293T cells were transfected with vectors carrying the following combinations of expression cassettes (x marks the presence of the respective expression cassette in the combination):

| | combinations | | | | | |
|---|---|---|---|---|---|---|
| expression cassette | 1 | 2 | 3 | 4 | 5 | 6 |
| NLS-iRFP-Flag-GFP^{Y39->TAG}_ | X | X | X | X | X | X |
| 6His | | | | | | |
| HA-PKR(2-174) | X | X | X | X | X | X |
| CMV-tetO-NES-PylRS^{AF} | | | X | X | | X |
| CMV-NES-PylRS^{AF} | X | X | | | X | |
| T7-tRNA^{Pyl}-HDV | | | | X | X | X |
| 8xtetO-U6-tetO-tRNA^{Pyl} | X | | X | | | |
| H1-tetO-tRNA^{Pyl} | | X | | | | |
| CMV-tetO-T7RNAP | | | | X | X | |
| CMV-NES-T7RNAP | | | | | | X |
| results shown in FIG. | 5a | 5b | 5c | 5d | 5e | 5f |

Controls were prepared in the same manner but without the expression cassette for HA-PKR(2-174).

Tetracycline was added for expression of tet-inducible elements and the cells were fed with the ncAA BOC as described in section C) above. Control samples were prepared identically with the exception that the addition of one or both of tetracycline and Boc was omitted.

The cells were analyzed by FCS as described in section D) above. The results shown in FIG. 5 indicate that co-expression of PKR(2-174) increases the intensity of GFP fluorescence detected in double positive cells, indicating the presence of an increased amount of full-length reporter gene expression product (resulting from successful amber suppression) per cell.

### Abbreviations

BCN=2-amino-6-(9-biocyclo[6.1.0]non-4-ynylmethoxycarbonylamino)hexanoid acid
BOC=2-amino-6-(tert-butoxycarbonylamino)hexanoic acid, in the examples “BOO” specifically designates (2S)-2-amino-6-(tert-butoxycarbonylamino)hexanoic acid=Boc-L-Lys-OH═N-α-tert-butyloxycarbonyl-L-lysine
CMV=cytomegalovirus
Crm1=chromosomal region maintenance 1, also known as karyopherin exportin 1
dsRNA=double-stranded RNA
dSTORM=direct stochastic optical reconstruction microscopy
E. coli BL21(DE3)AI=E. coli strain B F⁻ ompT gal dcm lon hsdS_{B}(r_{B}⁻m_{B}⁻) λ(DE3 [lacl lacUV5-T7p07 ind1 sam7 nin5]) [malB⁺]_{K-12}(λ^{S}) araB::T7RNAP-tetA
eIF-2α=alpha subunit of eukaryotic initiation factor 2
FBS=fetal bovine serum
FISH=fluorescence in situ hybridization
GCE=genetic code expansion
GFP=green fluorescent protein
glmS=glucosamine-6-phosphate synthetase
H1 promoter=human RNA polymerase III promoter
HDV=hepatitis delta virus
IPTG=isopropyl β-D-1-thiogalactopyranoside
iRFP=infrared fluorescent protein
ncAA=non-canonical amino acid
NES=nuclear export signal
NLS=nuclear localization signal
O-tRNA=orthogonal tRNA
O-RS=orthogonal RS
PAINT=point accumulation for imaging in nanoscale topography
PBS=phosphate buffered saline
PKR=protein kinase R; alternative names: dsRNA-dependent protein kinase, interferon-(IFN-)inducible dsRNA-activated serine/threonine-protein kinase, p68 kinase
PMSF=phenylmethyAsulfonyl fluoride
POI=polypeptide of interest, target polypeptide
PrK=2-amino-6-(prop-2-ynoxycarbonylamino)hexanoic acid
pRS=prokaryotic RS
ptRNA=prokaryotic tRNA
ptRNA-ribozyme=prokaryotic RNA molecule comprising a ptRNA and at least one ribozyme
PylRS=pyrrolysyl tRNA synthetase
PylRS^{AF}=mutant M. mazei pyrrolysyl tRNA synthetase comprising amino acid substitutions Y306A and Y384F
RP-HPLC=reversed phase high-performance liquid chromatography
RS=aminoacyl tRNA synthetase
RT=room temperature
rtTA=reverse tTA
SCO=2-amino-6-(cyclooct-2-yn-1-yloxycarbonylamino)hexanoic acid
SPAAC=(copper-free) strain promoted alkyne-azide cycloaddition
SPIEDAC=(copper-free) strain promoted inverse-electron-demand Diels-Alder cycloaddition
SRM=super-resolution microscopy
SV40=simian vacuolating virus 40
T7RNAP=T7 RNA polymerase
TCO=2-amino-6[(2E-cyclooct-2-en-1-yl)oxycarbonylamino]hexanoic acid
TCO*=2-amino-6[(2E-cyclooct-2-en-1-yl)oxycarbonylamino]hexanoic acid
tetR=tetracycline repressor
tetO=tetracycline operator
tTA=tetracycline-controlled transcriptional activator
TRE=tetracycline-responsive promoter element (also termed tetracycline response element)
tRNA^{Pyl}=tRNA that can be acylated with pyrrolysin by a wild-type or modified PylRS and has an anticodon that, for site-specific incorporation of the ncAA into a POI, is preferably the reverse complement of a selector codon. (in the tRNA^{Pyl} used in the examples, the anticodon is CUA.)
U6 promoter=promoter that normally controls expression of the U6 RNA (a small nuclear RNA) in mammalian cells
VS=Varkud satellite

### Listing of Sequence

Listing of sequences

| | | | | |
|---|---|---|---|---|
| SEQ | | | | |
| ID | | | | |
| NO | Description | Source | Type | Features |

ribozymes

| | | | | |
|---|---|---|---|---|
| 1 | hepatitis delta virus | hepatitis delta | gcuagccauggucccagccuccucgcuggcggcuagugggcaacaugcuucggcauggcgaaugggac | HOV ribozyme core |
| | (HDV) ribozyme (Avis) | virus | | sequence(7-68) |
| | | | | flanking sequence (1-6) |
| | | | | (has to be there, but |
| | | | | sequence can be |
| | | | | different) |
| 2 | hepatitis delta virus | hepatitis delta | gggtcggcatggcatctccacctcctcgcggtccgacctgggctacttcggtaggctaagggagaag | |
| | (HDV) ribozyme | virus | | |
| | (Schuerer) | | | |
| 3 | hepatitis delta virus | hepatitis delta | gagggatagtacagagcctccccgtggctcccttggataaccaactgatactgtac | |
| | (HDV) ribozyme | virus | | |
| | (Akoopie) | | | |
| 4 | hammerhead ribozyme | artificial | ucacguuucccugaugaguccgugaggacgaaacgguacccgguaccguc | HH ribozyme core |
| | | sequence | | sequence(11-45) |
| | | synthetic | | flanking sequences |
| | | | | (1-10, 46-50) (have to |
| | | | | be there, but sequence |
| | | | | can be different) |
| 5 | hairpin ribozyme | artificial | ggagagagaagucaaccagagaaacacaccaacccauugcacuccggguuggugguauauuaccuggu | [There are other |
| | | sequence | acgggggaaacuucgugguggccg | hairpin sequences |
| | | synthetic | | existing, but this one |
| | | | | seems to be the most |
| | | | | used one.] |
| 6 | Varkud satellite (VS) | Neurospora | gggaaagcuugcgaagggcgucgucgccccgagcgguaguaagcagggaacucaccuccaauuucagu | |
| | ribozyme | intermedia | acugaaauugucguagcaguugacuacuguuaugugauugguagaggcuaagugacgguauuggcgua | |
| | | | agucaguauugcagcacagcacaagcccgcuugcgagaau | |
| 7 | glucosamine-6- | Bacillus spec. | gcgccagaactggcaccauugcacuccggugccaguugacgaggugggguuuaucgagauuucggcgg | |
| | phosphate synthetase | | augacucccgguuguucaucacaaccgcaagcuuuuacuuaaaucauuaaggugacuuaguggacaaa | |
| | (glmS) riboswitch (glmS | | ggugaaagugugauga | |
| | ribozyme) | | | |

Elements of the TetOn/Off system

| | | | | |
|---|---|---|---|---|
| 8 | tetracycline operator | artificial | tccctatcagtgatagaga | |
| | (tetO) | sequence | | |
| | | synthetic | | |
| 9 | minimal cytomegalovirus | artificial | gtaggcgtgtacggtgggaggtctatataagcagagctcgtttagtgaaccgtcagatc | |
| | (CMV) promoter | sequence | | |
| | | derived from | | |
| | | CMV | | |
| | | promoter | | |
| 10 | “Tet-On” promoter | artificial | caattgctcgagtttactccctatcagtgatagagaacgtatgtcgactttactccctatcagtgata | tetQ sequences (8x, |
| | comprising 8 tetO | sequence | gagaacgtatgaagagtttactccctatcagtgatagagaacgtatgcagactttactccctatcagt | 18-36, 54-72, 90-108, |
| | sequences | synthetic | gatagagaacgtataaggagtttactccctatcagtgatagagaacgtatgaccagtttactccctat | 126-144, 162-180, 198- |
| | | | cagtgatagagaacgtatctacagtttactccctatcagtgatagagaacgtatatccagtttactcc | 216, 234-252, 270-288) |
| | | | ctatcagtgatagagaacgtatacgctttaggcgtgtacggtgggcgcctataaaagcagagctcgtt | in CMV promoter |
| | | | tagtgaaccgtcagatcgcctggagcaattccacaacacttttgtcttataccaactttccgtaccac | |
| | | | ttcctaccctcgaaagtccatatggctagcctgcagggatccagatatcaagcttcacc | |
| 11 | “Tet-On” promoter | artificial | ctcgactttactccctatcagtgatagagaacgtatgaagagtttactccctatcagtgatagagaac | tetO sequences (7x, |
| | comprising 7 tetO | sequence | gtatgcagactttactccctatcagtgatagagaacgtataaggagtttactccctatcagtgataga | 12-30, 48-66, 84-102, |
| | sequences | synthetic | gaacgtatgaccagtttactccctatcagtgatagagaacgtatctacagtttactcccatcagtgat | 120-138, 156-174, |
| | | | agagaacgtatatccagtttactccctatcagtgatagagaacgtatacgctttaggcgtgtacggtg | 192-210, 228-246) in |
| | | | ggcgcctataaaagcagagctcgtttagtgaaccgtcagatcgcctggagcaattccacaacactttt | CMV promoter |
| | | | gtcttataccaactttccgtaccacttcctaccctcgaaagtccatatggctagcctgcagggatcca | |
| | | | gatatcaagcttcacc | |
| 12 | “T-REX” promoter | artificial | gacattgattattgactagttattaatagtaatcaattacggggtcattagttcatagcccatatatg | CMV enhancer (1-380) |
| | comprising two tetO | sequence | gagttccgcgttacataacttacggtaaatggcccgcctggctgaccgcccaacgacccccgcccatt | CMV promoter (381-584) |
| | sequences | synthetic | gacgtcaataatgacgtatgttcccatagtaacgccaatagggactttccattgacgtcaatgggtgg | tetO sequences |
| | | | agtatttacggtaaactgcccacttggcagtacatcaagtgtatcatatgccaagtacgccccctatt | (586-604, 607-625) |
| | | | gacgtcaatgacggtaaatggcccgcctggcattatgcccagtacatgaccttatgggactttcctac | (2x) |
| | | | ttggcagtacatctacgtattagtcatcgctattaccatcgtggtgcggttttggcagtacatcaatg | |
| | | | ggcgtggatagcggtttgactcacggggatttccaagtctccaccattgacgtcaatgggagtttgtt | |
| | | | ttggcaccaaaatcaacgggactttccaaaatgtcgtaacaactccgccccattgacgcaaatgggcg | |
| | | | gtaggcgtgtacggtgggaggtctatataagcagagctctccctatcagtgatagagatctccctatc | |
| | | | actgatagagatcgtcgacgagctcgtttagtgaaccgtcagatcgcctggagacgccatccacgctg | |
| | | | ttttgacctccatagaagacaccgggaccgatccagcctccggactctagcgtttaaacatcacaagt | |
| | | | tgtacaaaaaagcaggcgctagcgtttaaacttaagcttcacc | |
| 13 | tetracycline repressor | artificial | MSRLDKSKVINSALELLNEVGIEGLTTRKLAQKLGVEQPTLYWHVKNKRALLDALAIEMLDRHHTH | |
| | (tetR) UniProtKB/ | sequence | FCPLEGESWQDFLRNNAKSFRCALLSHRDGAKVHLGTRPTEKQYETLENQLAFLCQQGFSLENA | |
| | Swiss-Prot accession | synthetic | LYALSAVGHFTLGCVLEDQEHQVAKEERETPTTDSMPPLLRQAIELFDHQGAEPAFLFGLELIICG | |
| | no.: P04483.1 | | LEKQLKCESGS | |
| 14 | rtTA expression | artificial | DNA: | CMV |
| | cassette | sequence | gttgacattgattattgactagttattaatagtaatcaattacggggtcattagttcatagcccatat | TetOn3G |
| | | synthetic | atggagttccgcgttacataacttacggtaaatggcccgcctggctgaccgcccaacgacccccgccc | |
| | | | attgacgtcaataatgacgtatgttcccatagtaacgccaatagggactttccattgacgtcaatggg | |
| | | | tggactatttacggtaaactgcccacttggcagtacatcaagtgtatcatatgccaagtacgccccct | |
| | | | attgacgtcaatgacggtaaatggcccgcctggcattatgcccagtacatgaccttatgggactttcc | |
| | | | tacttggcagtacatctacgtattagtcatcgctattaccatggtgatgcggttttggcagtacatca | |
| | | | atgggcgtggatagcggtttgactcacggggatttccaagtctccaccccattgacgtcaatgggagt | |
| | | | ttgttttggcaccaaaatcaacgggactttccaaaatgtcgtaacaactccgccccattgacgcaaat | |
| | | | gggcggtaggogtgtacggtgggaggtctatataagcagagctctctggctaactagagaacccactg | |
| | | | cttactggcttatcgaaattaatacgactcactatagggagacccaagctggctagcgtttaaactta | |
| | | | agcttctcgagcaccatgtctagactggacaagagcaaagtcataaactctgctctggaattactcaa | |
| | | | tggagtcggtatcgaaggcctgacgacaaggaaactcgctcaaaagctgggagttgagcagcctaccc | |
| | | | tgtatactggcacgtgaagaacaagcgggccctgctcgatgccctgccaatcgagatgctggacaggc | |
| | | | atcatacccactcctgccccctggaaggcgagtcatggcaagactttctgcggaacaacgccaagtca | |
| | | | taccgctgtgctctcctctcacatcgcgacggggctaaagtgcatctcggcacccgcccaacagagaa | |
| | | | acagtacgaaaccctggaaaatcagctcgcgttcctgtgtcagcaaggcttctccctggagaacgcac | |
| | | | tgtacgctctgtccgccgtgggccactttacactgggctgcgtattggaggaacaggagcatcaagta | |
| | | | gcaaaagaggaaagagagacacctaccaccgattctatgcccccacttctgaaacaagcaattgagct | |
| | | | gttcgaccggcagggagccgaacctgccttccttttcggcctggaactaatcatatgtggcctggaga | |
| | | | aacagctaaagtgcgaaagcggcgggccgaccgacgcccttgacgattttgacttagacatgctccca | |
| | | | gccgatgcccttgacgactttgaccttgatatgctgccgctgacgctcttgacgattttgaccttgac | |
| | | | atgctcccccgggtaa | |
| 52 | TetOn3G protein | human | Protein: | |
| | Zhou et al, Nature, | immunodeficiency | MSRLDKSKVINSALELLNGVGIEGLTTRKLAQKLGVEQPTLYWHVKNKRALLDALPIEMLDRHHTH | |
| | 2006 | virus | SCPLEGESWQDFLRNNAKSYRCALLSHRDGAKVHLGTRPTEKQYETLENQLAFLCQQGFSLENA | |
| | | (HIV)-rtTA | LYALSAVGHFTLGCVLEEQEHQVAKEERETPTTDSMPPLLKQAIELFDRQGAEPAFLFGLELIICGL | |
| | | variant, | EKQLKCESGGPTDALDDFDLDMLPADALDDFDLDMLPADALDDFDLDMLPG* | |
| | | optimized for | | |
| | | TetOn-system | | |

T7 promoter/RNA polymerase

| | | | | |
|---|---|---|---|---|
| 15 | T7 promoter class III | artificial | taatacgactcactatag | G at position 18 is |
| | (core) | sequence | | preferred nucleotide |
| | | derived from | | at the +1 position to |
| | | enterobacteria | | promote efficient |
| | | phage T7 | | transcription |
| | | promoter | | initiation |
| 16 | T7 promoter class III | artificial | taatacgactcactatagg | GG at positions 18 and |
| | | sequence | | 19 are preferred |
| | | derived from | | nucleotides at the +1 |
| | | enterobacteria | | and +2 positions to |
| | | phage T7 | | promote efficient |
| | | promoter | | transcription |
| | | | | initiation |
| 17 | T7 promoter class II | artificial | taatacgactcactatta | A at position 18 is |
| | (core) | sequence | | preferred nucleotide |
| | | derived from | | at the +1 position of |
| | | enterobacteria | | the gene to promote |
| | | phage T7 | | efficient |
| | | promoter | | transcription |
| | | | | initiation |
| 18 | T7 RNA polymerase | Enterobacteria | MNTINIAKNDFSDIELAAIPFNTLADHYGERLAREQLALEHESYEMGEARFRKMFERQLKAGEVAD | |
| | | phage T7 | NAAAKPLITTLLPKMIARINDWFEEVKAKRGKRPTAFQFLQEIKPEAVAYITIKTTLACLTSADNTTV | |
| | | | QAVASAIGRAIEDEARFGRIRDLEAKHFKKNVEEQLNKRVGHVYKKAFMQVVEADMLSKGLLGGE | |
| | | | AWSSWHKEDSIHVGVRCIEMLIESTGMVSLHRQNAGVVGQDSETIELAPEYAEAIATRAGALAGIS | |
| | | | PMFQPCVVPPKPWTGITGGGYWANGRRPLALVRTHSKKALMRYEDVYMPEVYKAINIAQNTAW | |
| | | | KINKKVLAVANVITKWKHCPVEDIPAIEREELPMKPEDIDMNPEALTAWKRAAAAVYRKDKARKSR | |
| | | | RISLEFMLEGANKFANHKAIWFPYNMDWRGRVYAVSMFNPQGNDMTKGLLTLAKGKPIGKEGYY | |
| | | | WLKIHGANCAGVDKVPFPERIKFIEENHENIMACAKSPLENTWWAEQDSPFCFLAFCFEYAGVQH | |
| | | | HGLSYNCSLPLAFDGSCSGIQHFSAMLRDEVGGRAVNLLPSETVQDIYGIVAKKVNEILQADAING | |
| | | | TDNEWVTVIDENTGEISEKVKLGTKALAGQWLAYGVTRSVTKRSVMTLAYGSKEFGFRQQVLED | |
| | | | TIQPAIDSGKGLMFTQPNQAAGYMAKLIWESVSVTVVAAVEAMNWLKSAAKLLAAEVKDKKTGEI | |
| | | | LRKRCAVHWVTPDGFPVWQEYKKPIQTRLNLMFLGQFRLQPTINTNKDSEIDAHKQESGIAPNFV | |
| | | | HSQDGSHLRKTVVWAHEKYGIESFALIHDSFGTIPADAANLFKAVRETMVDTYESCDVLADFYDQ | |
| | | | FADQLHESQLDKMPALPAKGNLNLRDILESDFAFA | |
| 19 | NES-T7 RNA | artificial | MCPVPLQLPPLERLTLDNTINIAKNDFSDIELAAIPFNTLADHYGERLAREQLALEHESYEMGEARF | NES |
| | polymerase | sequence | RKMFERQLKAGEVADNAAAKPLITTLLPKMIARINDWFEEVKAKRGKRPTAFQFLQEIKPEAVAYIT | |
| | | derived from | IKTTLACLTSADNTTVQAVASAIGRAIEDEARFGRIRDLEAKHFKKNVEEQLNKRVGHVYKKAFMQ | |
| | | Enterobacteria | WVEADMLSKGLLGGEAWSSWHKEDSIHVGVRCIEMLIESTGMVSLHRQNAGVVGQDSETIELAP | |
| | | phage T7 | EYAEAIATRAGALAGISPMFQPCVVPPKPWTGITGGGYWANGRRPLALVRTHSKKALMRYEDVY | |
| | | | MPEVYKAINIAQNTAWKINKKVLAVANVITKWKHCPVEDIPAIEREELPMKPEDIDMNPEALTAWK | |
| | | | RAAAAVYRKDKARKSRRISLEFMLEQANKFANHKAIWFPYNMDWRGRVYAVSMFNPQGNDMTK | |
| | | | GLLTLAKGKPIGKEGYYWLKIHGANCAGVDKVPFPERIKFIEENHENIMACAKSPLENTWWAEQD | |
| | | | SPFCFLAFCFEYAGVQHHGLSYNCSLPLAFDGSCSGIQHFSAMLRDEVGGRAVNLLPSETVQDIY | |
| | | | GIVAKKVNEILQADAINGTDNEVVTVTDENTGEISEKVKLGTKALAGQWLAYGVTRSVTKRSVMTL | |
| | | | AYGSKEFGFRQQVLEDTIQPAIDSGKGLMFTQPNQAAGYMAKLIWESVSVTVVAAVEAMNWLKS | |
| | | | AAKLLAAEVKDKKTGEILRKRCAVHWVTPDGFPVWQEYKKPIQTRLNLMFLGQFRLQPTINTNKD | |
| | | | SEIDAHKQESGIAPNFVHSQDGSHLRKTWWWAHEKYGIESFALIHDSFGTIPADAANLFKAVRETM | |
| | | | VDTYESCDVLADFYDQFADQLHESQLDKMPALPAKGNLNLRDILESDFAFA | |
| 20 | NLS consensus | artificial | KX¹X²X³ | X¹ and X³ are |
| | | sequence | | independently selected |
| | | synthetic | | from K and R, and X² |
| | | | | is any naturally |
| | | | | occurring amino acid |
| 21 | NLS of SV40 large T | simian | PKKKRKV | |
| | antigen | vacuolating | | |
| | | virus 40 | | |
| | | (SV40) | | |

pRS and ptRNA

| | | | | |
|---|---|---|---|---|
| 22 | Methanosarcina mazei | artificial | DNA: | ATG (M¹) may be missing |
| | PylRS Y306A, Y384F | sequence | atggcgtgcccggtgccgctgcagctgccgccgctggaacgcctgaccctggatgataaaaaaccgct | |
| | mutant (PylRS^{AF}) | synthetic | gaataccctgatctctgctactggtctgtggatgagtcgtaccggaaccattcataaaatcaaacacc | |
| | | | acgaggttagccgttcgaaaatctatattgagatggcgtgtggcgatcatctggttgtgaacaatagc | |
| | | | cgctcttctcgtacagcacgtgcactgcgtcaccacaaatatcgtaaaacctgtaaacgttgccgtgt | |
| | | | gtccgatgaggatctgaacaaattcctgacaaaagccaatgaggaccaaacaagcgtgaaagtgaaag | |
| | | | tcgttagcgctcctacccgtactaaaaaagcaatgccgaaatccgttgctcgtgcccctaaaccactg | |
| | | | gaaaacactgaagcagcacaggcacagccgtctggaagcaaattctctccggccattcctgtttctac | |
| | | | ccaggagtccgtttctgttccagcaagtgtgagcaccagcattagcagtattagcaccggtgccaccg | |
| | | | ctagcgccctggttaaaggcaataccaatccgattacaagcatgtctgccccggttcaagcatcagct | |
| | | | ccagcactgacaaaatcccaaaccgatcgtctggaggttctgctgaatccgasagacgaaatcagcct | |
| | | | gaattccggcaaaccgtttcgtgaactggagagcgaactgctgtcacgtcgtaaaaaagacctgcaac | |
| | | | aaatctatgccgaagaacgtgagaactatctggggaaactggaacgtgaaatcacccgctttttcgtg | |
| | | | gatcgtggctttctggagatcaaatccccgattctgattcctctggagtatatcgagcgtatgggcat | |
| | | | cgacaatgataccgaactgagcaaacaaattttccgtgtggataaaaacttctgtctgcgccctatgc | |
| | | | tagcaccaaatctggctaactatctgcgcaaactggaccgtgccctgcctgatcctatcaaaatcttc | |
| | | | gagatcggcccgtgttatcgtaaagagtccgacggtaaagaacatctggaggagtttaccatgctgaa | |
| | | | cttttgccaaatgggttcaggttgtactcgtgagaacctggaaagcatcatcaccgattttctgaacc | |
| | | | acctgggcattgacttcaaaattgtgggcgacagctgtatggtgtttggcgacaccctggatgtcatg | |
| | | | cacggcgacctggaactgtctagtgccgttgtgggcccaatcccgctggatcgtgagtggggtatcga | |
| | | | caaaccttggatcggtgcgggttttggtctggagcgtctgctgaaagtaaaacacgacttcaagaaca | |
| | | | tcaaacgtgctgcacgttcgagtcctattacaatggtatttctactaacctgtaa | |
| 53 | Methanosarcina mazei | artificial | Protein: | M¹ may be missing |
| | PylRS Y306A, Y384F | sequence | MACPVPLqLPPLERLTLDDKKPLNTLISATGLWMSRTGTIHKIKHHEVSRSKIYIEMACGDHLVVN | Y306A, Y384F |
| | mutant (PylRS^{AF})_ | synthetic | NSRSSRTARALRHHKYRKTCKRCRVSDEDLNKFLTKANEDQTSVKVKVVSAPTRTKKAMPKSVA | |
| | amino acid sequence | | RAPKPLENTEAAQAQPSGSKFSPAIPVSTQESVSVPASVSTSISSISTGATASALVKGNTNPITSM | |
| | | | SAPVQASAPALTKSQTDRLEVLLNPKDEISLNSGKPFRELESELLSRRKKDLQQIYAEERENYLGK | |
| | | | LEREITRFFVDRGFLEIKSPILIPLEYIERMGIDNDTELSKQIFRVDKNFCLRPMLAPNLANYLRKLDR | |
| | | | ALPDPIKIFEIGPCYRKESDGKEHLEEFTMLNFCQMGSGCTRENLESITDFLNHLGIDFKIVGDSC | |
| | | | MVFGDTLDVMHGDLELSSAVVGPIPLDREWGIDKPWIGAGFGLERLLKVKHDFKNIKRAARSESY | |
| | | | YNGISTNL | |
| 23 | Methanosarcina mazei | artificial | DNA: | ATG (M¹) may be missing |
| | PylRS N346A, C348A | sequence | atggcgtgcccggtgccgctgcagctgccgccgctggaacgcctgaccctggatgacaaaaaaccgct | |
| | mutant (PylRS^{AA}) | synthetic | gaataccctgatctctgctactggtctgtggatgagtcgtaccggaaccattcataaaatcaaacacc | |
| | | | acgaggttagccgttcgaaaatctatattgagatggcgtgtggcgatcatctggttgtgaacaatagc | |
| | | | cgctcttctcgtacagcacgtgcactgcgtcaccacaaatatcgtaaaacctgtaaacgttgccgtgt | |
| | | | gtccgatgagatctgaaaattcctgacaaaagccaatgaggaagcgtgaaagtgaaagtcgttagcgc | |
| | | | tcctacccgtactaaaaaagcaatgccgaaatccgttgctcgtgcccctaaaccactggaaaacactg | |
| | | | aagcagcacaggcacagccgtctggaagcaaattctctccggccattcctgtttctacccaggagtcc | |
| | | | gtttctgttccagcaagtgtgagcaccagcattagcagtattagcaccggtgccaccgctagcgccct | |
| | | | ggttaaaggcaataccaatccgattacaagcatgtctgccccggttcaagcatcagctccagcactga | |
| | | | caaaatcccaaaccgatcgtctggggttctgctgaatccgaaagacgaaatcagcctgaattccggca | |
| | | | aaccgtttcgtgaactggagagcgaactgctgtcacgtcgtaaaaaagacctgcaacaaatctatgcc | |
| | | | gaagaacgtgagaactatctggggaaactggaacgtgaaatcacccgctttttcgtggatcgtggctt | |
| | | | tctggagatcaaatccccgattctgattcctctggagtatatcgagcgtatgggcatcgacaatgata | |
| | | | ccgaactgagcaaacaaattttccgtgtggataaaaacttctgtctgcgccctatgctggcaccaaat | |
| | | | ctgtataactatctgcgcaaactggaccgtgccctgcctgatcctatcaaaatcttcgagatcggccc | |
| | | | gtgttatcgtaaagagtccgacggtaaagaacatctggaggagtttaccatgctggcctttgcccaaa | |
| | | | tgggttcaggttgtactcgtgagaacctggaaagcatcatcaccgattttctgaaccacctgggcatt | |
| | | | gacttcaaaattgtgggcgacagctgtatggtgtatggcgacaccctggatgtcatgcacggcgacct | |
| | | | ggaactgtctagtgccgttgttggaccaattccgctggaccgtgagtggggtatcgacaaaccgtgga | |
| | | | tcggagcaggattcggtctggaacgcctgctgaaagtgaaacacgacttcaaaaacatcaaacgtgcc | |
| | | | gcccgttctgaatcgtattataacgggatctctacgaacctgtaa | |
| 54 | Methanosarcina mazei | artificial | Protein: | M¹ may be missing |
| | PylRS N346A, C348A | sequence | MACPVPLqLPPLERLTLDDKKPLNTLISATGLWMSRTGTIHKIKHHEVSRSKIYIEMACGDHLVVN | N346A, C348A |
| | mutant (PylRS^{AA})_ | synthetic | NSRSSRTARALRHHKYRKTCKRCRVSDEDLNKFLTKANEDQTSVKVKVVSAPTRTKKAMPKSVA | |
| | amino acid sequence | | RAPKPLENTEAAQAQPSGSKFSPAIPVSTQESVSVPASVSTSISSISTGATASALVKGNTNPITSM | |
| | | | SAPVQASAPALTKSQTDRLEVLLNPKDEISLNSGKPFRELESELLSRRKKDLQQIYAEERENYLGK | |
| | | | LEREITRFFVDRGFLEIKSPILIPLEYIERMGIDNDTELSKQIFRVDKNFCLRPMLAPNLYNYLRKLQR | |
| | | | ALPDPIKIFEIGPCYRKESDGKEHLEEFTMLAFAQMGSGCTRENLESIITDFLNHLGIDFKIVGDSC | |
| | | | MVYGdTLDVMHGDLELSSAVVGPIPLDREWGIDKPWIGAGFGLERLLKVKHDFKNIKRAARSESY | |
| | | | YNGISTNL | |
| 24 | Methanosarcina mazei | artificial | gcgtgcccggtgccgctgcagctgccgccgctggaacgcctgaccctggatgataaaaaaccgctgaa | |
| | PylRS Y306A, N346A, | sequence | taccctgatctctgctactggtctgtggatgagtcgtaccggaaccattcataaaatcaaacaccacg | |
| | C348A, Y384Fmutant | synthetic | aggttagccgttcgaaaatctatattgagatggcgtgtggcgatcatctggttgtgaacaatagccgc | |
| | (PylRS^{AAAF}) | | tcttctcgtacagcacgtgcactgcgtcaccacaaatatcgtaaaacctgtaaacgttgccgtgtgtc | |
| | | | cgatgaggatctgaacaaattcctgacaaaagccaatgaggaccaaacaagcgtgaaagtgaaagtcg | |
| | | | ttagcgctcctacccgtactaaaaaagcaatgccgaaatccgttgctcgtgcccctaaaccactggaa | |
| | | | aacactgaagcagcacaggcacagccgtctggaagcaaattctctccggccattcctgtttctaccca | |
| | | | ggagtccgtttctgttccagcaagtgtgagcaccagcattagcagtattagcaccggtgccaccgcta | |
| | | | gcgccctggttaaaggcaataccaatccgattacaagcatgtctgccccggttcaagcatcagctcca | |
| | | | gcactgacaaaatcccaaaccgatcgtctggaggttctgctgaatccgaaagacgaaatcagcctgaa | |
| | | | ttccggcaaaccgtttcgtgaactggagagcgaactgctgtcacgtcgtaaaaaagacctgcaacaaa | |
| | | | tctatgccgaagaacgtgagaactatctggggaaactggaacgtgaaatcacccgctttttcgtggat | |
| | | | cgtggctttctggagatcaaatccccgattctgattcctctggagtatatcgagcgtatgggcatcga | |
| | | | caatgataccgaactgagcaaacaaattttccgtgtggataaaaacttctgtctgcgccctatgctag | |
| | | | caccaaatctggctaactatctgcgcaaactggaccgtgccctgcctgatcctatcaaaatcttcgag | |
| | | | atcggcccgtgttatcgtaaagagtccgacggtaaagaacatctggaggagtttaccatgctggcctt | |
| | | | tgcccaaatgggttcaggttgtactcgtgagaacctggaaagcatcatcaccgattttctgaaccacc | |
| | | | tgggcattgacttcaaaattgtcggcgacagctgtatggtgtttggcgacaccctggatgtcatgcac | |
| | | | ggcgacctggaactgtctagtgccgttgtgggcccaatcccgctggatcgtgagtggggtatcgacaa | |
| | | | accttggatcggtgcgggttttggtctggagcgtctgctgaaagtaaaacacgacttcaagaacatca | |
| | | | aacgtgctgcacgttccagtcctattacaatggtatttctactaacctgtaa | |
| 55 | Methanosarcina mazei | artificial | Protein: | May be optionally |
| | PylRS Y306A, N346A, | sequence | ACPVPLQLPPLERLTLDDKKPLNTLISATGLWMSRTGTIHKIKHHEVSRSKIYIEMACGDHLVVNNS | extended by a M¹ |
| | C348A, Y384Fmutant | synthetic | RSSRTARALRHHKYRKTCKRCRVSDEDLNKFLTKANEDQTSVKVKVVSAPTRTKKAMPKSVARA | (before first A-amino |
| | (PylRS^{AAA}) amino acid | | PKPLENTEAAQAQPSGSKFSPAIPVSTQESVSVPASVSTSISSISTGATASALVKGNTNPITSMSAP | acid) Y306A, N346A, |
| | sequence | | VQASAPALIKSQTDRLEVLLNPKDEISLNSGKPFRELESELLSRRKKDLQQIYAEERENYLGKLER | C348A, Y384F |
| | | | EITRFFVDRGFLEIKSPILIPLEYIERMGIDNDTELSKQIFRVDKNFCLRPMLAPNLANYLRKLDRALP | |
| | | | DPIKIFEIGPCYRKESDGKEHLEEFTMLAFAQMGSGCTRENLESIITDFLNHLGIDFKIVGDSCMVF | |
| | | | GDTLdVMHGDLELSSAVVGPIPLDREWGIDKPWIGAGFGLERLLKVKHDFKNIKRAARSESYYNG | |
| | | | ISTNL | |
| 25 | pyrrolysyl tRNA of | artificial | ggaaacctgatcatgtagatcgaatggactctaaatccgttcagccgggttagattcccggggtttccg | Underlined: anticodon |
| | Methanosarcina mazei | sequence | | (31-33) |
| | for Amber codon | synthetic | | |
| | (tRNA^{Pyl,CUA}) | | | |
| 26 | pyrrolysyl tRNA of | artificial | ggaaacctgatcatgtagatcgaatggacttcaaatccgttcagccgggttagattcccggggtttccg | Underlined: anticodon |
| | Methanosarcina mazei | sequence | | (31-33) |
| | for Opal codon | synthetic | | |
| | (tRNA^{Pyl,UCA}) | | | |
| 27 | pyrrolysyl IRNA of | artificial | ggaaacctgatcatgtagatcgaatggactttaaatccgttcagccgggttagattcccggggtttccg | Underlined: anticodon |
| | Methanosarcina mazel | sequence | | (31-33) |
| | for Ochre codon | synthetic | | |
| | (tRNA^{Pyl,UUA}) | | | |

NES

| | | | | |
|---|---|---|---|---|
| 28 | NES motif | artificial | XXXXXX | Underlined: |
| | | sequence | | independently selected |
| | | synthetic | | from Leu, Ile, Val |
| | | | | Phe and Met |
| | | | | Bold: independently |
| | | | | selected from any |
| | | | | canonical amino acid |
| 29 | NES motif | artificial | XXXXXXX | Underlined: |
| | | sequence | | independently selected |
| | | synthetic | | from Leu, Ile, Val |
| | | | | Phe and Met |
| | | | | Bold: independently |
| | | | | selected from any |
| | | | | canonical amino acid |
| 30 | NES motif | artificial | LXXXXXLXX | Underlined: |
| | | sequence | | independently selected |
| | | synthetic | | selected from Leu, |
| | | | | Ile, Val, Phe and Met |
| | | | | Bold: independently |
| | | | | selected from any |
| | | | | canonical amino acid |
| | | | | Italic: independently |
| | | | | selected from Leu and |
| | | | | Ile |
| 31 | NES motif | artificial | LXXXXXLX | Underlined: |
| | | sequence | | independently |
| | | synthetic | | selected from Leu, |
| | | | | Ile, Val, Phe and Met |
| | | | | Bold: independently |
| | | | | selected from any |
| | | | | canonical amino acid |
| 32 | NES motif | artificial | LXXXXXXLX | Underlined: |
| | | sequence | | independently |
| | | synthetic | | selected from Leu, |
| | | | | Ile, Val, Phe and Met |
| | | | | Bold: independently |
| | | | | selected from any |
| | | | | canonical amino acid |
| 33 | NES motif | artificial | LXXXXXXXLXX | Underlined: |
| | | sequence | | Independently |
| | | synthetic | | selected from Leu, |
| | | | | Ile, Val, Phe and Met |
| | | | | Bold: independently |
| | | | | selected from any |
| | | | | canonical amino acid |
| | | | | Italic: independently |
| | | | | selected from Leu and |
| | | | | Ile |
| 34 | NES motif | artificial | LPPLERLTL | |
| | | sequence | | |
| | | synthetic | | |
| 35 | NES motif | artificial | ACPVPLQLPPLERLTLD | |
| | | sequence | | |
| | | synthetic | | |

dsRNA-binding polypeptides

| | | | | |
|---|---|---|---|---|
| 36 | HA-PKR(2-174) | artificial | MYPYDVPDYAAGDLSAGFFMEELNTYRQKQGVVLKYQELPNSGPPHDRRFTFQVIIDGREFPEG | HA (2-10) |
| | | sequence | EGRSKKEAKNAAAKLAVEILNKEKKAVSPLLLTTTNSSEGLSMGNYIGLINRIAQKKRLTVNYEQCA | human PKR(2-174) |
| | | synthetic | SGVHGPEGFHYKCKMGQKEYSIGTGSTKQEAKQLAAKLAYLQILSEETSVKS | (11-183) |
| 37 | core of dsRNA-binding | artificial | FMEELNTYRQKQGVVLKYQELPNSGPPHDRRFTFQVIIDGREFPEGEGRSKKEAKNAAAKLAVEI | conserved; R30, F32, |
| | domain of PKR | sequence | LNKEKKAVSPLLLTTTNSSEGLSMGNYIGLINRIAQKKRLTVNYEQCASGVHGPEGFHYKCKMGQ | G48, S50, K51, K52, |
| | | synthetic | KEYSIGTGSTKQEAKQLAAKLAYLQILSE | K55, F122, K141, K145 |
| | | | | restricted: |
| | | | | 1) R9, Q10 ≠ |
| | | | | hydrophobic (i.e.: |
| | | | | K, R, H, D, E, S, T, |
| | | | | N, Q, C, |
| | | | | preferably, |
| | | | | R9 = R, S, C, Q, H, |
| | | | | (K), Q10 = Q, (N) |
| | | | | 2) A59, L66, A149 ≠ |
| | | | | A59D, L66A, A149D |
| | | | | (i.e.: A59 = A, G, |
| | | | | I, L, M, V; L66 = |
| | | | | L, I, V, M; A149 = |
| | | | | A, G, I, L, M, V) |
| 38 | (full) dsRNA-binding | artificial | AGDLSAGFFMEELNTYRQKQGVVLKYQELPNSGPPHDRRFTFQVIIDGREFPEGEGRSKKEAKN | |
| | domain of PKR-M1 | sequence | AAAKLAVEILNKEKKAVSPLLLTTTNSSEGLSMGNYIGLINRIAQKKRLTVNYEQCASGVHGPEGF | |
| | | synthetic | HYKCKMGQKEYSIGTGSTKQEAKQLAAKLAYLQILSEETSVKS | |

constructs used in examples

| | | | | |
|---|---|---|---|---|
| 39 | expression cassette | artificial | ggcattgattattgactagttattaatagtaatcaattacggggtcattagttcatagcccatatatg | Promoter (1-852) |
| | for reporter gene | sequence | gagttccgcgttacataacttacggtaaatggcccgcctggctgaccgcccaacgacccccgcccatt | NLS (949-984) |
| | NLS-iRFP-Flag- | synthetic | gacgtcaataatgacgtatgttcccatagtaacgccaatagggactttccattgacgtcaatgggtgg | IRFP (988-1929) |
| | GFP^{Y39→TAG}-6His | | agtatttacggtaaactgcccacttggcagtacatcaagtgtatcatatgccaagtccgccccctatt | 25 Helix (1930-2004) |
| | | | gacgtcaatgacggtaaatggcccgcctggcattatgcccagtacatgaccttacgggactttcctac | Flag (2011-2034) |
| | | | ttggcagtacatctacgtattagtcatcgctattaccatggtgatgcggttttggcagtacaccaatg | GFP^{Y39} CDS (2035-2748) |
| | | | ggcgtggatagcggtttgactcacggggatttccaagtctccaccccattgacgtcaatgggagtttg | Y39-encoding triplet |
| | | | ttttggcaccaaaatcaacgggactttccaaaatgtcgtaataaccccgccccgttgacgcaaatggg | (2149-1251) |
| | | | cggtaggcgtgtacggtgggaggtctatataagcagagctcgtttagtgaaccgtcagatcactagaa | 6His (2749-2766) |
| | | | gctttattgcggtagtttatcacagttaaattgctaacgcagtcagtgcttctgacacaacagtctcg | |
| | | | aacttaagctgcagaagttggtcgtgaggcactgggcaggtaagtatcaaggttacaagacaggttta | |
| | | | aggagaccaatagaaactgggcttgtcgagacagagaagactcttgcgtttctgataggcacctattg | |
| | | | gtcttactgacatccactttgcctttctctccacaggtgtccactcccagttcaattacagctcttaa | |
| | | | ggctagagtacttaatacgactcactataggctagccaccatgggccgcctggaaagcaccccgccga | |
| | | | aaaaaaaacgcaaagtggaagatagcgcgagcgcggaaggatccgtcgccaggcagcctgacctcttg | |
| | | | acctg | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 40 | T7 RNA polymerase | artificial | [symbol] | CMV promoter (2-588) |
| | expression cassette | sequence | [symbol] | CDS (690-3341) |
| | for constitutive | synthetic | [symbol] | |
| | expression | | [symbol] | |
| | via CMV promoter | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 41 | NES-T7 RNA | artificial | [symbol] | Promoter (1-584) |
| | polymerase expression | sequence | [symbol] | tetO (586-604; |
| | cassette containing | synthetic | [symbol] | 607-625) |
| | “T-REx” promoter | | [symbol] | NES (798-845) |
| | comprising two tetO | | [symbol] | CDS (846-3494) |
| | sequences | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 42 | NES-T7 RNA | artificial | [symbol] | Promoter (1-458) |
| | polymerase expression | sequence | [symbol] | tetO (18-36; 54-72; |
| | cassette containing | synthetic | [symbol] | 90-108; 126-144; |
| | “Tet-On” promoter | | [symbol] | 162-180; 198-216; |
| | comprising 8 tetO | | [symbol] | 234-252: 270-288) |
| | sequences | | [symbol] | NES (473-520) |
| | | | [symbol] | CDS (521-3169) |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 43 | NES-17 RNA | artificial | [symbol] | CMV promoter (1-588) |
| | polymerase expression | sequence | [symbol] | NES (693-740) |
| | cassette for | synthetic | [symbol] | CDS (741-3389) |
| | constitutive | | [symbol] | |
| | expression via CMV | | [symbol] | |
| | promoter | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 44 | PylRS^{AF} expression | artificial | [symbol] | CMV promoter (1-588) |
| | cassette for | sequence | [symbol] | CDS (690-2054) |
| | constitutive | synthetic | [symbol] | 306A, 384F |
| | expression via CMV | | [symbol] | |
| | promoter | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 45 | NES-PylRS^{AF} expression | artificial | [symbol] | CMV promoter (1-588) |
| | cassette for | sequence | [symbol] | NES (696-743) |
| | constitutive | synthetic | [symbol] | CDS (744-2105) |
| | expression via CMV | | [symbol] | 306A, 384F |
| | promoter | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 46 | NES-PylRS^{AF} expression | artificial | [symbol] | Promoter (1-584) |
| | cassette containing | sequence | [symbol] | tetO (586-604; |
| | “T-REx” promoter | synthetic | [symbol] | 607-625) |
| | comprising two tetO | | [symbol] | NES (785-832) |
| | sequences | | [symbol] | CDS (833-2194) |
| | | | [symbol] | 306A, 384F |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 47 | NES-PyIRSAF expression | artificial | [symbol] | Promoter (1-458) |
| | cassette containing | sequence | [symbol] | tetO (18-36: 54-72; |
| | “Tet-On” promoter | synthetic | [symbol] | 90-108; 128-144; 162- |
| | comprising | | [symbol] | 180; 198-216; |
| | 8 tetO sequences | | [symbol] | 234-252; 270-288) |
| | | | [symbol] | NES (474-521) |
| | | | [symbol] | CDS (522-1883) |
| | | | [symbol] | 306A, 384F |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| 48 | tRNA^{Pyl} expression | artificial | [symbol] | T7 promoter (1-17) |
| | cassette for | sequence | [symbol] | tRNA^{Pyl} (18-86) |
| | expression | Synthetic | [symbol] | T7term (98-226) |
| | via T7 promoter | | | |
| 49 | tRNA^{Pyl}-HDV ribozyme | artificial | [symbol] | T7 promoter (1-17) |
| | expression cassette | sequence | [symbol] | tRNA^{Pyl} (18-86) |
| | for expression via T7 | synthetic | [symbol] | HDV (87-154) |
| | promoter | | [symbol] | T7term (167-295) |
| 50 | tRNA^{Pyl} expression | artificial | [symbol] | tetO sequences |
| | cassette containing a | sequence | [symbol] | (24-42; 60-78; 96- |
| | variant of the human U6 | synthetic | [symbol] | 114; 132-150; 168- |
| | promoter modified to | | [symbol] | 186; 204-222; 276- |
| | comprise 8 tetO before | | [symbol] | 294; 540-558; 568- |
| | and two tetO sequences | | [symbol] | 586) U6 promoter |
| | after the U6 promoter | | [symbol] | (337-637) |
| | | | [symbol] | tRNA^{Pyl} (589-657) |
| 51 | tRNA^{Pyl} expression | artificial | [symbol] | tetO sequences |
| | cassette containing a | sequence | [symbol] | (49-67; 77-95) |
| | variant of the human H1 | Synthetic | | H1 promoter (1-99) |
| | promoter modified to | | | tRNA^{Pyl} (100-168) |
| | comprise 2 tetO | | | |
| | sequences | | | |
| 56 | pCAGGS-T7-RNAP | artificial | [symbol] | CMV enhancer (1-380) |
| | | sequence | [symbol] | Chicken b-actin |
| | | synthetic | [symbol] | promoter (382-659) |
| | | | [symbol] | Chimeric intron |
| | | | [symbol] | chicken actin |
| | | | [symbol] | (661-1678) |
| | | | [symbol] | T7-RNAP (1789-4437) |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |
| | | | [symbol] | |

Further dsRNA-binding polypeptides

| | | | | |
|---|---|---|---|---|
| 57 | dsRNA-binding | Mus musculus | MASDTPGFYMDKLNKYRQMHGVAITYKELSTSGPPHDRRFTFQVLIDEKEFPEAKGRSKQEARN | |
| | polypeptide | | AAAKLAVDILDNENKVDCHTSASEQGLFVGNYIGLVNSFAQKKKLSVNYEQCEPNSELPQRFICK | |
| | (https://www.uniprot. | | CKIGQTMYGTGSGVTKQEAKQLAAKEAYOKLLKSPPKTAGTSSSVVTSTFSGFSSSSSMTSNGV | |
| | org/uniprot/Q03963) | | SQSAPGSFSSENVFTNGLGENKRKSGVKVSPDDVORNKYTLDARFNSDFEDIEEIGLGGFGQVF | |
| | | | KAKHRIDGKRYAIKRVKYNTEKAEHEVQALAELNHVNIVQYHSCWEGVDYDPEHSMSDTSRYKT | |
| | | | RCLFIQMEFCDKGTLEQWMRNRNQSKVDKALILDLYEQIVTGVEYIHSKGLIHRDLKPGNIFLVDE | |
| | | | RHIKIGDFGLATALENDGKSRTRRTGTLQYMSPEOLFLKHYGKEVDIFALGLILAELLHTCFTESEKI | |
| | | | KFFESLRKGDFSNDIFDNKEKSLLKKLLSEKPKDRPETSEILKTLAEWRNISEKKKRNTC | |
| 58 | dsRNA-binding | Pan | MAGDLSAGFFMEELNTYRQKOGVVLKYQELPNSGPPHDRRFTFRVIIDGREFPEGEGRSKKEAK | |
| | polypeptide | troglodytes | NAAAKLAVEILNKEKKAVSPLVLTTTNSSEGLSMGNYIGLINRIAQKKRLTVNYEQCASGVHGPEG | |
| | (https://www.uniprot. | | FHYKCKMGOKEYSIGTSSTKQEAKQLAAKLAYLQILSEETSVKSDYLSSGSFATTCDSQSNSLVT | |
| | org/uniprot/89UKH7) | | STLASESSSEGDFSADTSEINSNSDSLNSSSLLMNGLRNNORKAKRSLAPRFDLPDMKETKYTVD | |
| | | | KRFGMDFKEIELIGSGGFGQVFKAKHRIDGKTYVIKRVKYNNEKAEREVKALAKLDHVNIVHYNGC | |
| | | | WDGFDYDPETSDDDLESSDYDPENSKNSSRSKTKCLFIQMEFCDKGTLEQWIEDRRGKKLDKVL | |
| | | | ALELFEQITKGVDYIHSKKLIHRDLKPSNIFLVDTKOVKIGDFGLVTSLKNDGKRTRSKGTLRYMSP | |
| | | | EQISSQDYGKEVDLYALGLILAELLHVCDTAFETSKFFTDLRDGIISDILFDKKEKTLLQKLLSKKPE | |
| | | | DRPNTSEILRTLTVWKKSPEKNERHTC | |
| 59 | dsRNA-binding | Gorilla gorilla | MAGDLSAGFFMEELNTYRQKQGVVLKYQELPNSGPPHDRRFTFRVIIDEREFPEGEGRSKKEAK | |
| | polypeptide | | NAAAKLAVEILNKEKKAVSPLLLTTTNSSEGLSMGNYIGLINRIAQKKRLTVNYEQCASGVHGPEG | |
| | (https://www.uniprot. | | FHYKCKMGQKEYSIGTGSTKQEAKQLAAKLAYLQIVSEETSVKSDYLSSGSFATTCDSQSNSLVT | |
| | org/uniprot/G3RTN4) | | STLASESSSEGDFSADTSEINSNSDSLNSSSLLMNGLRNNQRKAKRSLAPRFDLPDMKETKYTVD | |
| | | | KRFGMDFKEIELIGSGGFGQVFKAKHRIDGKTYVIKRVKYNNEKAEREVKALAKLDHVNIVHYNGC | |
| | | | WDGVDYDPETSDDYLESSDYDPENSKNSSRSKTKCLFIQMEFCDKGTLEQWIENRRGEKLDKVL | |
| | | | ALELFEQITKGVDYIHSKKLIHRDLKPSNIFLVDTKQVKIGDFGLVTSLKNDGKRTRSKGTLRYMSP | |
| | | | EQISSQDYGKEVDLYALGLILAELLHVCDTAFETSKFFTDLRDGIISDIFDKKEKTLLOKLLSKKPED | |
| | | | RPNTSEILRTLTVWKKSPEKNERHTC | |
| 60 | pyrrolysyl tRNA of | Artificial | ggaaacctgatcatgtagatcgaatggactnnnaatccgttcagccgggttagattcccggggtttccg | Underlined: |
| | Methanosarcina mazei | sequence | | unidentified |
| | for unidentified codon | Synthetic | | anticodon (31-33) |
| | (tRNA^{Pyl,rem}) | cf.: SEQ ID | | |
| | | NOs: 25, 26 | | |
| | | and 27 | | |

[symbol]

indicates data missing or illegible when filed

1. A eukaryotic cell comprising:
(a) a polynucleotide encoding a prokaryotic aminoacyl tRNA synthetase (pRS),
(b) a polynucleotide encoding a prokaryotic tRNA (ptRNA) and one or more than one ribozyme,
wherein the pRS is capable of acylating the ptRNA, and
wherein the sequences encoding the ptRNA and the ribozyme(s) are linked such that transcription produces an RNA molecule comprising both the ptRNA and the ribozyme(s) (ptRNA-ribozyme).

2. The eukaryotic cell of claim 1, wherein the ribozyme is selected from a hammerhead ribozyme, a hairpin ribozyme, a Varkud satellite ribozyme, a glucosamine-6-phosphate synthetase riboswitch and a hepatitis delta virus (HDV) ribozyme.

3. The eukaryotic cell of claim 1, wherein the ribozyme is a HDV ribozyme which, in the ptRNA-ribozyme, is directly linked to the 3′ end of the ptRNA.

4. The eukaryotic cell of claim 1, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein expression of said RNA polymerase is controlled by a TRE.

5. The eukaryotic cell of claim 1, wherein the transcription of the pRS-encoding sequence is controlled by a tetracycline-responsive promoter element (TRE).

6. The eukaryotic cell of claim 4, further comprising a polynucleotide encoding a protein which binds to a tetracycline operator (tetO) sequence (tetO-binding protein), wherein the tetO-binding protein is selected from the group consisting of:
(i) a tetracycline repressor (tetR),
(ii) a tetracycline-controlled transcriptional activator (tTA), and
(iii) a reverse tTA (rtTA).

7. The eukaryotic cell of claim 1, wherein:
(i) transcriptional synthesis of the ptRNA-ribozyme is controlled by a T7 promoter, and/or
(ii) transcription of the pRS-encoding sequence is controlled by a T7 promoter,
and wherein the eukaryotic cell further comprises
(c) a polynucleotide encoding a T7 RNA polymerase (T7RNAP).

8. The eukaryotic cell of claim 7, wherein expression of the T7RNAP is controlled by a TRE.

9. The eukaryotic cell of claim 7, wherein the T7RNAP carries
(i) a nuclear localization signal (NLS), or
(ii) a nuclear export signal (NES), or
(iii) none of NES and NLS.

10. The eukaryotic cell of claim 1, further comprising a polynucleotide encoding a dsRNA-binding polypeptide.

11. A polynucleotide encoding an RNA molecule comprising a ptRNA and one or more than one ribozyme (ptRNA-ribozyme) as defined in claim 1, wherein:
a) the ribozyme is a HDV ribozyme which, in the ptRNA-ribozyme, is directly linked to the 3′ end of the ptRNA; or
b) the ptRNA is derived from a Methanosarcina species, in particular a pyrrolysyl tRNA of M. mazei; or
c) wherein a ptRNA molecule is encoded missing the 3′-terminal aminoacyl acceptor stem motif CCA; or
d) the ptRNA is encoded by the following nucleotide sequence (SEQ ID NO:60) ggaaacctgatcatgtagatcgaatggactnnnaatccgttcagccgggttagattcccggggtttccg wherein nnn encodes an anticodon.

12. A polynucleotide, or combination of two or more polynucleotides, comprising a nucleotide sequence encoding an RNA molecule comprising a ptRNA and one or more than one ribozyme (ptRNA-ribozyme) as defined in claim 1, and:
(i) a nucleotide sequence encoding a tetO-binding protein selected from the group consisting of: (a) a tetracycline repressor (tetR), (b) a tetracycline-controlled transcriptional activator (tTA), and (c), a reverse tTA (rtTA), or
(ii) a nucleotide sequence encoding a pRS as defined in claim 1, or
(ii) both of (i) and (ii).

13. A method for preparing a polypeptide of interest (POI) having one or more than one non-canonical amino acid (ncAA) residue in its amino acid sequence, wherein the method comprises:
(a) expressing, in a eukaryotic cell:
a prokaryotic aminoacyl tRNA synthetase (pRS) and
an RNA molecule comprising a prokaryotic tRNA (ptRNA) and one or more than one ribozyme (ptRNA-ribozyme);
and concomitantly or sequentially
(b) expressing the POI in the eukaryotic cell in the presence of one or more than one ncAA or salt thereof corresponding to the one or more than one ncAA residue of the POI; and
(c) optionally recovering the expressed POI;
wherein the pRS is capable of acylating the ptRNA with the ncAA or the salt thereof,
wherein the POI is encoded by a nucleotide sequence that comprises one or more than one selector codon encoding the one or more than one ncAA residue, and
wherein the selector codon is the reverse complement of the anticodon of the ptRNA.

14. The method of claim 13, wherein the transcriptional synthesis of the ptRNA-ribozyme is controlled by a T7 promoter, and wherein the method further comprises expressing a T7 RNA polymerase (T7RNAP) in the eukaryotic cell prior to or concomitantly with the thereby controlled expression of the ptRNA-ribozyme (i) or the pRS (ii) or both (iii), respectively.

15. The method of claim 13, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein the expression of said RNA polymerase is controlled by a TRE;
wherein the method further comprises expressing a tetracycline repressor (tetR) in the eukaryotic cell at least prior to the expression of the ptRNA-ribozyme, and
wherein expression of the ptRNA-ribozyme involves contacting the eukaryotic cell with tetracycline, doxycycline, or a functional analogue thereof, thereby inducing transcriptional synthesis of the ptRNA-ribozyme.

16. The method of claim 13, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a tetracycline repressor (tetR) in the eukaryotic cell at least prior to the expression of the pRS; and
wherein expression of the pRS involves contacting the eukaryotic cell with tetracycline, doxycycline, or a functional analogue thereof, thereby inducing transcription of the pRS-encoding sequence.

17. The method of claim 15, wherein:
(i) transcriptional synthesis of the ptRNA-ribozyme is controlled by a T7 promoter, or
(ii) transcription of the pRS-encoding sequence is controlled by a T7 promoter, or
(iii) both of (i) and (ii);
and wherein the method further comprises:
expressing a T7 RNA polymerase (T7RNAP) in the eukaryotic cell prior to or concomitantly with the thereby controlled expression of the ptRNA-ribozyme (i) or the pRS (ii) or both (iii), respectively, wherein expression of the T7RNAP is controlled by a TRE, and
expressing a tetR in the eukaryotic cell at least concomitantly with the expression of the T7RNAP;
wherein expression of the T7RNAP involves contacting the eukaryotic cell with tetracycline, doxycycline, or a functional analogue thereof, thereby inducing transcription of the T7RNAP-encoding sequence.

18. The method of claim 13, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein expression of said RNA polymerase is controlled by a TRE;
wherein the method further comprises:
(1) expressing a tetracycline-controlled transcriptional activator (tTA) in the eukaryotic cell prior to and concomitantly with the expression of the ptRNA-ribozyme, and
(2) keeping the eukaryotic cell prior to the expression of the ptRNA-ribozyme in contact with tetracycline, doxycycline, or a functional analogue thereof, thereby suppressing the transcriptional synthesis of the ptRNA-ribozyme, and then reducing the concentration of, or preferably removing, said tetracycline, doxycycline, or functional analogue thereof so as to induce transcriptional synthesis of the ptRNA-ribozyme.

19. The method of claim 13, wherein:
(i) transcription of the pRS-encoding sequence is controlled by a TRE, or
(ii) transcription of the pRS-encoding sequence is catalyzed by an RNA polymerase, wherein the expression of said RNA polymerase is controlled by a TRE;
wherein the method further comprises:
(1) expressing a tetracycline-controlled transcriptional activator (tTA) in the eukaryotic cell prior to and concomitantly with the expression of the pRS, and
(2) keeping the eukaryotic cell prior to the expression of the pRS in contact with tetracycline, doxycycline, or a functional analogue thereof, thereby suppressing transcription of the pRS-encoding sequence, and then reducing the concentration of, or preferably removing, said tetracycline, doxycycline, or functional analogue thereof so as to induce transcription of the pRS-encoding sequence.

20. The method of claim 18, wherein:
(i) transcriptional synthesis of the ptRNA-ribozyme is controlled by a T7 promoter, or
(ii) transcription of the pRS-encoding sequence is controlled by a T7 promoter, or
(iii) both of (i) and (ii);
wherein the method further comprises:
(3) expressing a T7 RNA polymerase (T7RNAP) in the eukaryotic cell prior to or concomitantly with the thereby controlled expression of the ptRNA-ribozyme (i) or the pRS (ii) or both (iii), respectively,
wherein expression of the T7RNAP is controlled by a TRE,
(4) expressing a tTA in the eukaryotic cell prior to and concomitantly with the expression of the T7RNAP, and
(5) keeping the eukaryotic cell prior to the expression of the T7RNAP in contact with tetracycline, doxycycline, or a functional analogue thereof, thereby suppressing transcription of the T7RNAP-encoding sequence, and then reducing the concentration of, or preferably removing, said tetracycline, doxycycline, or functional analogue thereof so as to induce transcription of the T7RNAP-encoding sequence.

21. The method of claim 13, wherein:
(i) transcriptional synthesis of the ptRNA-ribozyme is controlled by a tetracycline-responsive promoter element (TRE), or
(ii) the ptRNA-ribozyme is transcriptionally synthesized by an RNA polymerase, wherein the expression of said RNA polymerase is controlled by a TRE;
wherein the method further comprises expressing a reverse tTA (rtTA) in the eukaryotic cell at least concomitantly with the expression of the ptRNA, and
wherein expression of the ptRNA-ribozyme involves contacting the eukaryotic cell with doxycycline, or a functional analogue thereof, thereby inducing transcriptional synthesis of the ptRNA-ribozyme.

22. The method of claim 13, wherein:
(i) transcription of the pRS-encoding sequence is controlled by a tetracycline-responsive promoter element (TRE), or
(ii) transcription of the pRS-encoding sequence is catalyzed by an RNA polymerase, wherein the expression of said RNA polymerase is controlled by a TRE;
wherein the method further comprises expressing a reverse tTA (rtTA) in the eukaryotic cell at least concomitantly with the expression of the pRS; and
wherein expression of the pRS involves contacting the eukaryotic cell with doxycycline, or a functional analogue thereof, thereby inducing transcription of the pRS-encoding sequence.

23. A kit for preparing a polypeptide of interest (POI) having one or more than one non-canonical amino acid (ncAA) residue in its amino acid sequence, wherein the kit comprises one or more than one ncAA, or salt thereof, corresponding to the one or more than one ncAA residue of the POI, and:
(a) a polynucleotide of claim 11.

24. A kit for preparing a polypeptide of interest (POI) having one or more than one non-canonical amino acid (ncAA) residue in its amino acid sequence, wherein the kit comprises one or more than one ncAA, or salt thereof, corresponding to the one or more than one ncAA residue of the POI, and:
(a) a polynucleotide, or combination of two or more polynucleotides, of claim 12, each in combination with
(b) a polynucleotide encoding a dsRNA-binding polypeptide.

25. A kit for preparing a polypeptide of interest (POI) having one or more than one non-canonical amino acid (ncAA) residue in its amino acid sequence, wherein the kit comprises one or more than one ncAA, or salt thereof, corresponding to the one or more than one ncAA residue of the POI, and an eukaryotic cell of claim 1.
